SEQUENCE LISTING (275 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
GFTFRDYA

SEQ ID NO: 4 - protein, 8 aa, synthetic construct
ISGSGGNT

SEQ ID NO: 5 - protein, 18 aa, synthetic construct
AKDRLSITIRPRYYGLDV

SEQ ID NO: 6 - protein, 11 aa, synthetic construct
QSLLYSIGYNY

SEQ ID NO: 7 - skipped

SEQ ID NO: 8 - protein, 9 aa, synthetic construct
MQALQTPYT

SEQ ID NO: 9 - protein, 452 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 207 aa, synthetic construct
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 13 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 14 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 15 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 16 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 17 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 18 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 19 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 20 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 21 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 22 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 23 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 24 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 25 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 26 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 27 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 28 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 29 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 30 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 31 - protein, 109 aa, synthetic construct
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 32 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 35 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 36 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 37 - protein, 115 aa, synthetic construct
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, synthetic construct
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 40 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 41 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 45 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 46 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 47 - protein, 115 aa, synthetic construct
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, synthetic construct
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 51 - protein, 115 aa, synthetic construct
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 52 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 53 - protein, 115 aa, synthetic construct
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 54 - protein, 115 aa, synthetic construct
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, synthetic construct
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 57 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 58 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 59 - protein, 115 aa, synthetic construct
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 63 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 64 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 65 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 66 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 67 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 68 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 69 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 70 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 71 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 72 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 73 - protein, 116 aa, synthetic construct; identical to SEQ ID NO: 69

SEQ ID NO: 74 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 75 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 76 - protein, 111 aa, synthetic construct; identical to SEQ ID NO: 74

SEQ ID NO: 77 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 78 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 79 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 80 - protein, 111 aa, synthetic construct; identical to SEQ ID NO: 74

SEQ ID NO: 81 - protein, 116 aa, synthetic construct; identical to SEQ ID NO: 79

SEQ ID NO: 82 - protein, 111 aa, synthetic construct; identical to SEQ ID NO: 78

SEQ ID NO: 83 - protein, 116 aa, synthetic construct; identical to SEQ ID NO: 69

SEQ ID NO: 84 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 116 aa, synthetic construct; identical to SEQ ID NO: 75

SEQ ID NO: 86 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 116 aa, synthetic construct; identical to SEQ ID NO: 87

SEQ ID NO: 90 - protein, 111 aa, synthetic construct; identical to SEQ ID NO: 84

SEQ ID NO: 91 - protein, 116 aa, synthetic construct; identical to SEQ ID NO: 75

SEQ ID NO: 92 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 93 - protein, 116 aa, synthetic construct; identical to SEQ ID NO: 69

SEQ ID NO: 94 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 95 - protein, 116 aa, synthetic construct; identical to SEQ ID NO: 69

SEQ ID NO: 96 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 97 - protein, 116 aa, synthetic construct; identical to SEQ ID NO: 69

SEQ ID NO: 98 - protein, 111 aa, synthetic construct; identical to SEQ ID NO: 78

SEQ ID NO: 99 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 100 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 101 - protein, 116 aa, synthetic construct; identical to SEQ ID NO: 99

SEQ ID NO: 102 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 103 - protein, 116 aa, synthetic construct; identical to SEQ ID NO: 99

SEQ ID NO: 104 - protein, 111 aa, synthetic construct; identical to SEQ ID NO: 78

SEQ ID NO: 105 - protein, 116 aa, synthetic construct; identical to SEQ ID NO: 77

SEQ ID NO: 106 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, synthetic construct
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 108 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 109 - protein, 116 aa, synthetic construct; identical to SEQ ID NO: 77

SEQ ID NO: 110 - protein, 111 aa, synthetic construct
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 111 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 112 - protein, 111 aa, synthetic construct
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 113 - protein, 116 aa, synthetic construct
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 114 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 115 - protein, 116 aa, synthetic construct
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 116 - protein, 111 aa, synthetic construct
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 117 - protein, 116 aa, synthetic construct; identical to SEQ ID NO: 77

SEQ ID NO: 118 - protein, 111 aa, synthetic construct; identical to SEQ ID NO: 114

SEQ ID NO: 119 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 120 - protein, 111 aa, synthetic construct
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 121 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 122 - protein, 111 aa, synthetic construct
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 123 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 124 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 125 - protein, 116 aa, synthetic construct; identical to SEQ ID NO: 77

SEQ ID NO: 126 - protein, 111 aa, synthetic construct
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 127 - protein, 116 aa, synthetic construct; identical to SEQ ID NO: 77

SEQ ID NO: 128 - protein, 111 aa, synthetic construct
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 129 - protein, 116 aa, synthetic construct; identical to SEQ ID NO: 77

SEQ ID NO: 130 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 131 - protein, 116 aa, synthetic construct
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 132 - protein, 111 aa, synthetic construct
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 133 - protein, 116 aa, synthetic construct
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 134 - protein, 111 aa, synthetic construct; identical to SEQ ID NO: 114

SEQ ID NO: 135 - protein, 116 aa, synthetic construct; identical to SEQ ID NO: 77

SEQ ID NO: 136 - protein, 111 aa, synthetic construct; identical to SEQ ID NO: 124

SEQ ID NO: 137 - protein, 116 aa, synthetic construct; identical to SEQ ID NO: 107

SEQ ID NO: 138 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 139 - protein, 116 aa, synthetic construct
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 140 - protein, 111 aa, synthetic construct
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 141 - protein, 116 aa, synthetic construct; identical to SEQ ID NO: 107

SEQ ID NO: 142 - protein, 111 aa, synthetic construct; identical to SEQ ID NO: 114

SEQ ID NO: 143 - protein, 116 aa, synthetic construct; identical to SEQ ID NO: 113

SEQ ID NO: 144 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 145 - protein, 116 aa, synthetic construct; identical to SEQ ID NO: 77

SEQ ID NO: 146 - protein, 111 aa, synthetic construct; identical to SEQ ID NO: 138

SEQ ID NO: 147 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 148 - protein, 111 aa, synthetic construct
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 149 - protein, 116 aa, synthetic construct
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 150 - protein, 111 aa, synthetic construct
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 151 - protein, 116 aa, synthetic construct
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 152 - protein, 111 aa, synthetic construct
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 153 - protein, 116 aa, synthetic construct
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 154 - protein, 121 aa, synthetic construct
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 155 - protein, 127 aa, synthetic construct
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 156 - protein, 116 aa, synthetic construct
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 157 - protein, 116 aa, synthetic construct
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 158 - protein, 116 aa, synthetic construct
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 159 - protein, 116 aa, synthetic construct
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 160 - protein, 116 aa, synthetic construct
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 161 - protein, 116 aa, synthetic construct
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 162 - protein, 116 aa, synthetic construct
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 163 - protein, 116 aa, synthetic construct
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 164 - protein, 116 aa, synthetic construct
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 165 - protein, 116 aa, synthetic construct
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 166 - protein, 116 aa, synthetic construct
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 167 - protein, 116 aa, synthetic construct
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 168 - protein, 110 aa, synthetic construct
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 169 - protein, 110 aa, synthetic construct
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 170 - protein, 110 aa, synthetic construct
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 171 - protein, 110 aa, synthetic construct
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 172 - protein, 110 aa, synthetic construct
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 173 - protein, 110 aa, synthetic construct
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 174 - protein, 110 aa, synthetic construct
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 175 - protein, 110 aa, synthetic construct
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 176 - protein, 110 aa, synthetic construct
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 177 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 178 - protein, 110 aa, synthetic construct; identical to SEQ ID NO: 177

SEQ ID NO: 179 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 180 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 181 - protein, 110 aa, synthetic construct; identical to SEQ ID NO: 177

SEQ ID NO: 182 - protein, 111 aa, synthetic construct
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 183 - protein, 111 aa, synthetic construct
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 184 - protein, 111 aa, synthetic construct
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 185 - protein, 111 aa, synthetic construct
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 186 - protein, 111 aa, synthetic construct
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 187 - protein, 111 aa, synthetic construct
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 188 - protein, 117 aa, synthetic construct
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 189 - protein, 128 aa, synthetic construct
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 190 - protein, 122 aa, synthetic construct
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 191 - protein, 128 aa, synthetic construct
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 192 - protein, 117 aa, synthetic construct
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 193 - protein, 124 aa, synthetic construct
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 194 - protein, 117 aa, synthetic construct
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 195 - protein, 116 aa, synthetic construct
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 196 - protein, 117 aa, synthetic construct
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 197 - protein, 122 aa, synthetic construct
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 198 - protein, 107 aa, synthetic construct
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 199 - protein, 107 aa, synthetic construct
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 200 - protein, 107 aa, synthetic construct
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 201 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 202 - protein, 107 aa, synthetic construct
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 203 - protein, 107 aa, synthetic construct
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 204 - protein, 107 aa, synthetic construct
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 205 - protein, 107 aa, synthetic construct
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 206 - protein, 107 aa, synthetic construct
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 207 - protein, 107 aa, synthetic construct
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

SEQ ID NO: 208 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVKVGYRGGMDVWGQGTTVTVSS

SEQ ID NO: 209 - protein, 120 aa, synthetic construct
EVQLLESGGGLVQPGGSLRLSCAASGFTLSSYAMHWVRQAPGKGLEYVSGISPSGSSTYYANSVKGRFTISRDNPKNTLFLQMSSLRAEDTAVYYCVRSKVRYRGGMDVWGQGTTVTVSS

SEQ ID NO: 210 - protein, 120 aa, synthetic construct
EVQLLESGGGLVQPGGSLRLSCAASGFTLSSYAMHWVRQAPGKGLEYVSGISPSGVSTYYANSVKGRFTISRDNPKNTLFLQMSSLRAEDTAVYYCVRVKVKYRGGMDVWGQGTTVTVSS

SEQ ID NO: 211 - protein, 120 aa, synthetic construct
EVQLLESGGGLVQPGGSLRLSCAASGFTLSSYAMHWVRQAPGKGLEYVSGISPTSGSTYYANSVKGRFTISRDNPKNTLFLQMSSLRAEDTAVYYCVRVKVRYRGGMDVWGQGTTVTVSS

SEQ ID NO: 212 - protein, 120 aa, synthetic construct
EVQLLESGGGLVQPGGSLRLSCAASGFTLSSYAMHWVRQAPGKGLEYVSGISPTGTSTYYANSVKGRFTISRDNPKNTLFLQMSSLRAEDTAVYYCVRVKGAYRGGMDVWGQGTTVTVSS

SEQ ID NO: 213 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKVAYRGGMDVWGQGTTVTVSS

SEQ ID NO: 214 - protein, 120 aa, synthetic construct
EVQLLESGGGLVQPGGSLRLSCAASGFTLSSYAMHWVRQAPGKGLEYVSGISPSSTSTYYANSVKGRFTISRDNPKNTLFLQMSSLRAEDTAVYYCVRVKVLYRGGMDVWGQGTTVTVSS

SEQ ID NO: 215 - protein, 120 aa, synthetic construct
EVQLLESGGGLVQPGGSLRLSCAASGFTLSSYAMHWVRQAPGKGLEYVSGISPSSASTYYANSVKGRFTISRDNPKNTLFLQMSSLRAEDTAVYYCVRVKSKYRGGMDVWGQGTTVTVSS

SEQ ID NO: 216 - protein, 120 aa, synthetic construct
EVQLLESGGGLVQPGGSLRLSCAASGFTLSSYAMHWVRQAPGKGLEYVSGISGNSASTYYANSVKGRFTISRDNPKNTLFLQMSSLRAEDTAVYYCVRVKLKYRGGMDVWGQGTTVTVSS

SEQ ID NO: 217 - protein, 120 aa, synthetic construct
EVQLLESGGGLVQPGGSLRLSCAASGFTLSSYAMHWVRQAPGKGLEYVSGISHSGTSTYYANSVKGRFTISRDNPKNTLFLQMSSLRAEDTAVYYCVRVRVLYRGGMDVWGQGTTVTVSS

SEQ ID NO: 218 - protein, 120 aa, synthetic construct; identical to SEQ ID NO: 210

SEQ ID NO: 219 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVFVRYRGGMDVWGQGTTVTVSS

SEQ ID NO: 220 - protein, 120 aa, synthetic construct
EVQLLESGGGLVQPGGSLRLSCAASGFTLSSYAMHWVRQAPGKGLEYVSGISPTSASTYYANSVKGRFTISRDNPKNTLFLQMSSLRAEDTAVYYCVRVKGRYRGGMDVWGQGTTVTVSS

SEQ ID NO: 221 - protein, 120 aa, synthetic construct
EVQLLESGGGLVQPGGSLRLSCAASGFTLSSYAMHWVRQAPGKGLEYVSGISPTGGSTYYANSVKGRFTISRDNPKNTLFLQMSSLRAEDTAVYYCVRVKGRYRGGMDVWGQGTTVTVSS

SEQ ID NO: 222 - protein, 120 aa, synthetic construct
EVQLLESGGGLVQPGGSLRLSCAASGFTLSSYAMHWVRQAPGKGLEYVSGISHSGNSTYYANSVKGRFTISRDNPKNTLFLQMSSLRAEDTAVYYCVRVKRRYRGGMDVWGQGTTVTVSS

SEQ ID NO: 223 - protein, 120 aa, synthetic construct
EVQLLESGGGLVQPGGSLRLSCAASGFTLSSYAMHWVRQAPGKGLEYVSGISPSSNSTYYANSVKGRFTISRDNPKNTLFLQMSSLRAEDTAVYYCVRVKVRYRGGMDVWGQGTTVTVSS

SEQ ID NO: 224 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKPAYRGGMDVWGQGTTVTVSS

SEQ ID NO: 225 - protein, 120 aa, synthetic construct
EVQLLESGGGLVQPGGSLRLSCAASGFTLSSYAMHWVRQAPGKGLEYVSGISYSSASTYYANSVKGRFTISRDNPKNTLFLQMSSLRAEDTAVYYCVRVKVRYRGGMDVWGQGTTVTVSS

SEQ ID NO: 226 - protein, 108 aa, synthetic construct
ETTLTQSPDTLPLSPGDRASLSCRASQSVSSAYLAWYQQKPGQAPRLLIYGTSRRATGVPGRFSGSGSGTDFTLTISRLEPEDFAVYYCQLYGSSSVTFGQGTKLEIK

SEQ ID NO: 227 - protein, 108 aa, synthetic construct
EIVLTQSPGTLSLSPGERATLSCRASQGISSAYLAWYQQKPGQAPRLLIYGTSRRATGIPDRFSGSGSGTDFTLTISRLEPEDFAVYYCQLYGATSVTFGQGTKLEIK

SEQ ID NO: 228 - protein, 108 aa, synthetic construct; identical to SEQ ID NO: 227

SEQ ID NO: 229 - protein, 108 aa, synthetic construct
EIVLTQSPGTLSLSPGERATLSCRASQSVSSAYLAWYQQKPGQAPRLLIYGTSRRATGIPDRFSGSGSGTDFTLTISRLEPEDFAVYYCQLYGASSVTFGQGTKLEIK

SEQ ID NO: 230 - protein, 108 aa, synthetic construct; identical to SEQ ID NO: 229

SEQ ID NO: 231 - protein, 108 aa, synthetic construct; identical to SEQ ID NO: 227

SEQ ID NO: 232 - protein, 108 aa, synthetic construct; identical to SEQ ID NO: 229

SEQ ID NO: 233 - protein, 108 aa, synthetic construct
EIVLTQSPGTLSLSPGERATLSCRASQSISTAYLAWYQQKPGQAPRLLIYGTSRRATGIPDRFSGSGSGTDFTLTISRLEPEDFAVYYCQLYGASSVTFGQGTKLEIK

SEQ ID NO: 234 - protein, 108 aa, synthetic construct
EIVLTQSPGTLSLSPGERATLSCRASQDISSAYLAWYQQKPGQAPRLLIYGTSRRATGIPDRFSGSGSGTDFTLTISRLEPEDFAVYYCQLYGATSVTFGQGTKLEIK

SEQ ID NO: 235 - protein, 108 aa, synthetic construct
EIVLTQSPGTLSLSPGERATLSCRASQDVSSAYLAWYQQKPGQAPRLLIYGTSRRATGIPDRFSGSGSGTDFTLTISRLEPEDFAVYYCQLYGATSVTFGQGTKLEIK

SEQ ID NO: 236 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGATSVTFGQGTKLEIK

SEQ ID NO: 237 - protein, 108 aa, synthetic construct
EIVLTQSPGTLSLSPGERATLSCRASQDASNAYLAWYQQKPGQAPRLLIYGTSRRATGIPDRFSGSGSGTDFTLTISRLEPEDFAVYYCQLYGSSSVTFGQGTKLEIK

SEQ ID NO: 238 - protein, 108 aa, synthetic construct
EIVLTQSPGTLSLSPGERATLSCRASQGVSSAYLAWYQQKPGQAPRLLIYGTSRRATGIPDRFSGSGSGTDFTLTISRLEPEDFAVYYCQLYGRSSVTFGQGTKLEIK

SEQ ID NO: 239 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGTSSVTFGQGTKLEIK

SEQ ID NO: 240 - protein, 108 aa, synthetic construct
EIVLTQSPGTLSLSPGERATLSCRASQSVSTAYLAWYQQKPGQAPRLLIYGTSRRATGIPDRFSGSGSGTDFTLTISRLEPEDFAVYYCQLYGATSVTFGQGTKLEIK

SEQ ID NO: 241 - protein, 108 aa, synthetic construct; identical to SEQ ID NO: 234

SEQ ID NO: 242 - protein, 108 aa, synthetic construct
EIVLTQSPGTLSLSPGERATLSCRASQGVSTAYLAWYQQKPGQAPRLLIYGTSRRATGIPDRFSGSGSGTDFTLTISRLEPEDFAVYYCQLYGATSVTFGQGTKLEIK

SEQ ID NO: 243 - protein, 108 aa, synthetic construct
EIVLPQSPGTLSLSPGERATLSCRASQGVSSAYLAWYQQKPGQAPRLLIYGTSRRATGIPDRFSGSGSGTDFTLTISRLEPEDFAVYYCQLYGSTSVTFGQGTKLEIK

SEQ ID NO: 244 - protein, 118 aa, synthetic construct
EVQLVESGGGLVQPKGSLKLSCAASGFTFNTYGMHWVRQAPGKGLEWVAHIRSKSSNYATYYADSVKDRFTISRDDSQSMLYLQMNNLKTEDTAMYYCVRWFRAMDYWGQGTSVTVSS

SEQ ID NO: 245 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMINLKTEDTAMYYCVRHRGGAWFAYWGQGTLVSVSA

SEQ ID NO: 246 - protein, 121 aa, synthetic construct
QVQLVETGGGLVRPGNSLKLSCVTSGFTFSNYRMHWLRQPPGKRLEWIAVITVKSNNYGANYAESVKGRFAISRDDSKSSVYLEMNRLREEDTATYFCSRERAYGNPFDYWGQGTTLTVSS

SEQ ID NO: 247 - protein, 118 aa, synthetic construct
EVQLVESGGGLVQPKGSLKLSCAASGFTFNMYAMNWVRQAPGQGLEWVARIRSKSNNYATYYADSVKDRFIISRDDSESMVYLQMSNLRAADTAMYYCVRHLRAMDYWGQGTSVTVSS

SEQ ID NO: 248 - protein, 118 aa, synthetic construct
EVQLVESGGGLVQPKGSLKLSCAASGFSFNMYAMNWVRQAPGKGLEWVARIRTKSNHYSTYYADSVKDRFTISRDDSASMFYLQMNNLKTEDTAMYFCVRHLRAMDYWGQGTSVTVSS

SEQ ID NO: 249 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMNNLKTEDTAMYYCVRHRGGAWFAYWGQGTLVTVSA

SEQ ID NO: 250 - protein, 118 aa, synthetic construct
EVQLVESGGGLVRPKGSLKLSCAASGFSFNTYAMNWVRQAPGKGLEWIVWIRSKSHNYATYYADSVKDRFTISRDDSESMLYLQMNNLKTEDTAMYYCVRHLRAMDYWGQGTSVTVSS

SEQ ID NO: 251 - protein, 118 aa, synthetic construct
EVRLVESGGGLVQPKGSLKLSCEASGFSFNMYAMNWVRQAPGKGLEWITHIRSKSNNYATYYADSVKDRFIISRDDSESMVYLQMNNLKTEDTAMYYCVRLLRALDYWGQGTSVTVSS

SEQ ID NO: 252 - protein, 118 aa, synthetic construct
EVQLVESGGGLVQPKGSLKLSCAASGFTFNMYGMHWVRQAPGKGLEWVAHIRSKSSNYATYYADSVKDRLTISRDDSQSMLYLQMNNLKTEDTAMYYCVRWFRAMDYWGQGTSVTVSS

SEQ ID NO: 253 - protein, 118 aa, synthetic construct
EVQLVESGGGLVKPGGSLRLSCAASGFTFSMYGMHWVRQAPGKGLEWVGHIRSKSSNYATYYADSVKDRFTISRDDSKNTLYLQMNSLKTEDTAVYYCTTWFRAMDYWGQGTLVTVSS

SEQ ID NO: 254 - protein, 118 aa, synthetic construct
EVQLVESGGGLVQPGGSLRLSCAASGFTFSMYGMHWVRQAPGKGLEWVSHIRSKSSNYATYYADSVKDRFTISRDNAKNSLYLQMNSLRAEDTAVYYCARWFRAMDYWGQGTLVTVSS

SEQ ID NO: 255 - protein, 118 aa, synthetic construct
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTLVTVSS

SEQ ID NO: 256 - protein, 118 aa, synthetic construct
EVQLVESGGGLVQPGGSLRLSCAASGFTFSMYGMHWVRQAPGKGLEWVGHIRSKSSNYATYYADSVKDRFTISRDDSKNSLYLQMNSLKTEDTAVYYCARWFRAMDYWGQGTLVTVSS

SEQ ID NO: 257 - protein, 118 aa, synthetic construct; identical to SEQ ID NO: 254

SEQ ID NO: 258 - protein, 118 aa, synthetic construct
EVQLVESGGGLEQPGGSLRLSCAGSGFTFRMYGMHWVRQAPGKGLEWVSHIRSKSSNYATYYADSVKDRFTISRDNSKNTLYLQMNSLRAEDTAVYYCAKWFRAMDYWGQGTTVTVSS

SEQ ID NO: 259 - protein, 118 aa, synthetic construct
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTTVTVSS

SEQ ID NO: 260 - protein, 107 aa, synthetic construct
DIVMTQSHKFMSTSVGDRVSITCKASQDVSTAVAWYQEKPGQSPKLLIYWASTRHTGVPDRFTGSGSGTDYTLTISSVQAEDLALYYCQQHYSTPLTFGAGTKLELK

SEQ ID NO: 261 - protein, 111 aa, synthetic construct
DIVVTQSPASLAVSLGQRATISCRASKSVSTSGYSYMHWYQQKPGQPPKLLIYLASNLESGVPARFSGSGSGTDFTLNIHPVEEEDVAIYYCQHSRELPLTFGAGTKLELK

SEQ ID NO: 262 - protein, 107 aa, synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYGSYPYTFGGGTKLEIK

SEQ ID NO: 263 - protein, 111 aa, synthetic construct
DIVLTQSPASLTVSLGQRATISCRASKSVSTSGYSYMHWYQQKPGQPPKLLIYLASNLESGVPARFSGSGSGTDFTLNIHPVEEEDAATYYCQHSRELPITFGSGTKLEIK

SEQ ID NO: 264 - protein, 111 aa, synthetic construct
DIVLTQSPASLVVSLGQRATISCRASQSVSTSGYSYMHWYQQKPGQPPKLLIYLASNVQSGVPARFSGSGSGTDFTLNIHPVEEEDVATYYCHHNRDLPFTFGSGTKLEIK

SEQ ID NO: 265 - protein, 111 aa, synthetic construct; identical to SEQ ID NO: 261

SEQ ID NO: 266 - protein, 111 aa, synthetic construct
DIVLTQSPASLAVSLGQRATISCRASKSVSASGYSYMHWYQQKPGQPPKLLIYLASNLQSGVPARFSGSGSGTDFTLNIHPVEEEDAATYYCQHSRELPPTFGGGTKLEIK

SEQ ID NO: 267 - protein, 111 aa, synthetic construct
DIVLTQSPASLAVFLGQRATISCRASKSVSTSGYSYMHWYQQKAGQPPKLLIYLASNLESGVPARFSGSGSGTDFTLNIHPVEEEDAATYYCHHSRELPITFGSGTKLEMK

SEQ ID NO: 268 - protein, 107 aa, synthetic construct; identical to SEQ ID NO: 260

SEQ ID NO: 269 - protein, 107 aa, synthetic construct
EIVLTQSPGTLSLSPGERATLSCKASQDVSTAVAWYQQKPGQAPRLLIYWASTRHTGIPDRFSGSGSGTDFTLTISRLEPEDFAVYYCQQHYSTPLTFGQGTKVEIK

SEQ ID NO: 270 - protein, 107 aa, synthetic construct
DIVMTQSPLSLPVTPGEPASISCKASQDVSTAVAWYLQKSGQSPQLLIYWASTRHTGVPDRFSGSGSGTDFTLKISRVEAEDVGFYYCQQHYSTPLTFGQGTKLEIK

SEQ ID NO: 271 - protein, 107 aa, synthetic construct
DIVMTQSPDSLAVSLGERATINCKASQDVSTAVAWYQQKPGQPPKLLIYWASTRHTGVPDRFSGSGSGTDFTLTISSLQAEDVAVYYCQQHYSTPLTFGGGTKVEIK

SEQ ID NO: 272 - protein, 107 aa, synthetic construct
DIQMTQSPSSLSASVGDRVTITCKASQDVSTAVAWYQQKPGKVPKLLIYWASTRHTGVPSRFSGSGSGTDFTLTISSLQPEDVATYYCQQHYSTPLTFGGGTKVEIK

SEQ ID NO: 273 - protein, 107 aa, synthetic construct
DIQLTQSPSFLSASVGDRVTITCKASQDVSTAVAWYQQKPGKAPKLLIYWASTRHTGVPSRFSGSGSGTEFTLTISSLQPEDFATYYCQQHYSTPLTFGGGTKVEIK

SEQ ID NO: 274 - protein, 107 aa, synthetic construct
DIQMTQSPSSLSASVGDRVTITCKASQDVSTAVAWYQQKPGKAPKLLLYWASTRHTGVPSRFSGSGSGTDYTLTISSLQPEDFATYYCQQHYSTPLTFGGGTKVEIK

SEQ ID NO: 275 - protein, 107 aa, synthetic construct
AIRMTQSPFSLSASVGDRVTITCKASQDVSTAVAWYQQKPAKAPKLFIYWASTRHTGVPSRFSGSGSGTDYTLTISSLQPEDFATYYCQQHYSTPLTFGGGTKVEIK

(19) United States
(12) Patent Application Publication
(10) Pub. No.: US 2024/0034798 A1
(43) Pub. Date: Feb. 1, 2024

(54) METHODS FOR TREATING EOSINOPHILIC ESOPHAGITIS BY ADMINISTERING AN IL-4R ANTAGONIST

(71) Applicant: REGENERON PHARMACEUTICALS, INC., Tarrytown, NY (US)

(72) Inventors: Jennifer D. HAMILTON, Ridgefield, CT (US); Mohamed KAMAL, Demarest, NJ (US); Matthew P. KOSLOSKI, Yorktown Heights, NY (US); Jennifer MALONEY, Hastings-on-Hudson, NY (US); Marcella RUDDY, Wellesley, MA (US); Arsalan Q. SHABBIR, Mount Kisco, NY (US); Allen RADIN, New York, NY (US)

(21) Appl. No.: 18/349,012

(22) Filed: Jul. 7, 2023

Related U.S. Application Data
(60) Provisional application No. 63/359,577, filed on Jul. 8, 2022.

(51) Int. Cl. C07K 16/28; A61P 37/08

(52) U.S. Cl. CPC C07K 16/2866; A61P 37/08; C07K 2317/565; A61K 2039/545

(57) ABSTRACT

Methods for treating eosinophilic esophagitis in a pediatric subject are provided. In one aspect, the methods comprise administering to the subject one or more doses of an interleukin-4 receptor (IL-4R) antagonist, such as an anti-IL-4R antibody or antigen-binding fragment thereof.

### REFERENCE TO A SEQUENCE LISTING XML

This application contains a Sequence Listing which has been submitted electronically in XML format. The Sequence Listing XML is incorporated herein by reference. Said XML file, created on Jun. 27, 2023, is named 40848_0116 USU1_SL.xml and is 267,877 in size.

### CROSS-REFERENCE TO RELATED APPLICATIONS

This application claims priority to U.S. Provisional Patent Application No. 63/359,577, filed on Jul. 8, 2022, the contents of which are incorporated by reference herein.

### BACKGROUND

Eosinophilic esophagitis (EoE) is a chronic inflammatory disease characterized by esophageal dysfunction and eosinophilic inflammation in the esophagus, and thought to be triggered by an abnormal type 2 immune response to food allergens (Furuta, et al., J Allergy Clin Immunol, 2017, 139:1131-1134; Liacouras, et al., J Allergy Clin Immunol, 2011, 128:3-20.e6). Although considered a rare disease, the current prevalence is estimated at 22.7 per 100,000 worldwide (Arias, et al., Aliment Pharmaco Ther, 2016, 43:3-15) and appears to be on the increase (Dellon, Gastroenterology Clinics of North America, 2014, 43:201-218). Eosinophilic esophagitis has been reported in all ages; however, most cases are in children and adults younger than 50 years. Children under the age of 18 represent approximately 30% of the EoE patient population (see, e.g., Dellon, et al., Clinical Gastroenterology and Hepatology, 2014, 12:589-596).

The primary clinical manifestations of EoE in both adults and children over 10 years of age are dysphagia and food impaction (Lucendo, et al., United European Gastroenterol J, 2017, Clinical features in younger children are non-specific in nature and vary significantly depending on the patient's age and ability of the patient to describe salient symptoms. Infants and toddlers are more likely to present with feeding difficulties, vomiting, or regurgitation with the potential for failure to thrive, whereas school-age children present with complaints of abdominal pain and heartburn (luliano, et al., Acta Biomed, 2018, 89:20-26). Older children with symptomatic EoE may also modify their dietary and eating behavior by taking small bites, chewing thoroughly, eating slowly, drinking copious fluids, and avoiding food consistencies that stick, which is highly suggestive of dysphagia, as this is a feeding behavior reported in adults as an attempt to prevent esophageal food impactions. Id. These symptoms lead to substantially impaired quality of life (see, e.g., DeBrosse, et al., J Allergy Clin Immunol, 2011, 128:132-138). Endoscopic findings are related to the inflammation in the esophagus and consist of fixed or transient concentric rings, longitudinal furrows, white plaques, reduced mucosal vascularity, fragile or crepe-like mucosa, and strictures. Furrows and white plaques are likely the most common finding in children, while rings are not common in children (Singla and Moawad, Clin Transl Gastroenterol, 2016, 7:e155). Some patients (particularly pediatric patients) may present with a normal-appearing esophagus, but still have histologically active EoE (Wechsler, et al., Clin Gastroenterol Hepatol, 2018, 16:1056-1063).

Current standard of care for EoE consists of diet adjustment (e.g., food-elimination diets), as well as off-label proton pump inhibitors (PPIs), off-label use of swallowed topical corticosteroids, and esophageal dilation. However, the standard therapies for EoE are limited by variable response rates, relapse after therapy cessation, and adverse effects on quality of life. Esophageal dilation is frequently utilized to relieve dysphagia symptoms caused by esophageal strictures, but because strictures do not occur as commonly in children, esophageal dilation is not as commonly performed in children with EoE as compared to in adults with EoE (Chehade, et al., J Allergy Clin Immunol Pract, 2018, 6:1534-1544.e5). Additionally, emergency endoscopy for prolonged and/or painful food impaction, and esophageal dilation to provide relief from strictures, are associated with a risk of severe esophageal injury and do not alter the underlying pathogenesis or progression of the disease. Diets eliminating specific foods can be effective in a significant percentage of patients; however, 30% to 40% of patients do not have resolution of disease with dietary modification (Nhu, et al., Curr Treat Options Gastroenterol, 2019, 17:48-62). Proton pump inhibitors can result in histologic remission in approximately 50% of patients with EoE (Lucendo, et al., United European Gastroenterol J, 2017, 5:335-358) with the remaining patients unresponsive. Swallowed topical corticosteroids have been reported in clinical trials to induce partial clinical responses and histologic remission; however, they are not uniformly effective and may be associated with local fungal infections, as well as a risk of growth suppression and hypothalamic-pituitary-adrenal axis suppression following systemic absorption (Golekoh, et al., J Pediatr, 2016, 170:240-245), limiting their use to short term. Thus, there remains a high unmet medical need for safe and effective therapies for treating EoE in children.

### SUMMARY

In one aspect, methods of treating, preventing, or ameliorating at least one symptom of eosinophilic esophagitis (EoE) in a pediatric subject<12 years of age are provided. In some embodiments, the method comprises administering to the subject one or more doses of an interleukin-4 receptor (IL-4R) antagonist. In some embodiments, the IL-4R antagonist specifically binds IL-4 and/or IL-13. In some embodiments, the IL-4R antagonist specifically binds IL-4Rα (e.g., human IL-4Rα).

In some embodiments, the IL-4R antagonist is an anti-IL-4R antibody, or an antigen-binding fragment thereof, that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence of LGS, and the LCDR3 comprises the amino acid sequence of SEQ ID NO:8.

In some embodiments, the subject is ≥1 year old and <12 years old. In some embodiments, the subject has a body weight≥5 kg. In some embodiments, the subject has a body weight<60 kg.

In some embodiments, the subject is <12 years old and has a body weight≥5 kg to <15 kg. In some embodiments, the subject is <12 years old and has a body weight≥15 kg to <30 kg. In some embodiments, the subject is <12 years old and has a body weight≥30 kg to <60 kg. In some embodiments, the subject is <12 years old and has a body weight≥40 kg.

In some embodiments, the subject is ≥1 year old and has a body weight≥5 kg to <15 kg. In some embodiments, the subject is ≥1 year old and has a body weight≥15 kg to <30 kg. In some embodiments, the subject is ≥1 year old and has a body weight≥30 kg to <60 kg. In some embodiments, the subject is ≥1 year old and has a body weight≥40 kg.

In some embodiments, prior to the onset of treatment with the IL-4R antagonist the subject has an intraepithelial eosinophilic infiltration peak cell count≥15 eos/hpf as measured by endoscopic biopsy in at least two of the proximal esophageal region, mid esophageal region, and distal esophageal region.

In some embodiments, the subject has been previously treated with a swallowed topical corticosteroid and/or a proton pump inhibitor (PPI). In some embodiments, the subject is unresponsive, inadequately responsive, or intolerant to treatment with a swallowed topical corticosteroid and/or a PPI, or standard of care treatment is contraindicated. In some embodiments, the subject has a concomitant atopic disease. In some embodiments, the concomitant atopic disease is a food allergy, atopic dermatitis, asthma, chronic rhinosinusitis, allergic rhinitis, or allergic conjunctivitis.

In some embodiments, prior to the onset of treatment the subject:
has a baseline peak intraepithelial eosinophilic cell count≥70 eos/hpf;
has a baseline mean intraepithelial eosinophilic cell count≥50 eos/hpf;
has a baseline EoE Endoscopic Reference Score (EoE-EREFS) score of at least 6; and/or
has a baseline serum total IgE level of at least 400 IU/L.

In some embodiments, the subject is selected on the basis of not exhibiting one or more exclusion criteria selected from the group consisting of:
(a) having a body weight<5 kg;
(b) having a body weight≥60 kg;
(c) having eosinophilic gastroenteritis, hypereosinophilic syndrome, or eosinophilic granulomatosis with polyangiitis (Churg-Strauss syndrome);
(d) having a history of Crohn's disease, ulcerative colitis, celiac disease, or prior esophageal surgery;
(e) having an esophageal stricture unable to be passed with a standard, diagnostic, upper endoscope or having an esophageal stricture that requires dilation;
(f) treatment with a swallowed topical corticosteroid within the previous 8 weeks;
(g) treatment with subcutaneous immunotherapy (SCIT) unless on a stable maintenance dose for at least 1 year;
(h) prior treatment with treatment with sublingual immunotherapy (SLIT), epicutaneous immunotherapy (EPIT), or oral immunotherapy (OIT);
(i) treatment with a systemic immunosuppressant or immunomodulating drug within the previous 3 months;
(j) initiation or change of a food-elimination diet regimen within the previous 6 weeks;
(k) initiation, discontinuation, or change in the dosage regimen of a PPI, leukotriene inhibitor, nasal corticosteroid, or inhaled corticosteroid within the previous 8 weeks;
(l) treatment with a live (attenuated) vaccine within the previous 4 weeks;
(m) having active H. pylori, a helminthic infection, an active parasitic infection, or a chronic or acute infection requiring treatment with systemic antibiotics, antivirals, or antifungals;
(n) having a known or suspected immunodeficiency disorder;
(o) having a hepatic disease; and
(p) having a platelet level<100×10³/μL, a neutrophil level 1.5×10³/μL, or an estimated glomerular filtration rate (eGFR)<30 mL/min/1.73 m².

In some embodiments, the anti-IL-4R antibody or antigen-binding fragment thereof comprises a heavy chain variable region (HCVR) comprising the amino acid sequence of SEQ ID NO:1 and comprises a light chain variable region (LCVR) comprising the amino acid sequence of SEQ ID NO:2. In some embodiments, the anti-IL-4R antibody comprises a heavy chain comprising the amino acid sequence of SEQ ID NO:9 and a light chain comprising the amino acid sequence of SEQ ID NO:10. In some embodiments, the IL-4R antagonist is dupilumab.

In some embodiments, the IL-4R antagonist is administered at a dose of about 50 mg to about 600 mg. In some embodiments, the IL-4R antagonist is administered at a dose of about 100 mg to about 300 mg every week or every two weeks. In some embodiments, the IL-4R antagonist is administered subcutaneously.

In some embodiments, the subject has a body weight≥5 kg, wherein:
for a subject weighing≥5 kg to <15 kg, the IL-4R antagonist is administered at a dose of about 100 mg Q2W, about 200 mg Q3W, or about 200 mg Q4W;
fora subject weighing≥15 kg to <30 kg, the IL-4R antagonist is administered ata dose of about 200 mg Q2W or about 300 mg Q4W;
for a subject weighing≥30 kg to <60 kg, the IL-4R antagonist is administered at a dose of about 300 mg Q2W or about 200 mg Q2W; and/or
for a subject weighing≥60 kg, the IL-4R antagonist is administered at a dose of about 300 mg QW or about 300 mg Q2W.

In some embodiments, the subject has a body weight≥5 kg, wherein:

for a subject weighing≥5 kg to <15 kg, the IL-4R antagonist is administered at a dose of about 100 mg Q2W or about 200 mg Q3W;
for a subject weighing≥15 kg to <30 kg, the IL-4R antagonist is administered ata dose of about 200 mg Q2W;
for a subject weighing≥30 kg to <60 kg, the IL-4R antagonist is administered at a dose of about 300 mg Q2W; and/or
for a subject weighing≥60 kg, the IL-4R antagonist is administered at a dose of about 300 mg QW.

In some embodiments, the subject has a body weight≥5 kg, wherein:

for a subject weighing≥5 kg to <15 kg, the IL-4R antagonist is administered at a dose of about 200 mg Q3W;
fora subject weighing≥15 kg to <30 kg, the IL-4R antagonist is administered at a dose of about 200 mg Q2W;
for a subject weighing≥30 kg to <40 kg, the IL-4R antagonist is administered at a dose of about 300 mg Q2W; and/or
for a subject weighing≥40 kg, the IL-4R antagonist is administered at a dose of about 300 mg QW.

In some embodiments, the subject weighs≥5 kg to <15 kg. In some embodiments, the subject weighs≥5 kg to <15 kg and the IL-4R antagonist is administered at a dose of about 100 mg Q2W. In some embodiments, the subject weighs≥5 kg to <15 kg and the IL-4R antagonist is administered at a dose of about 200 mg Q3W. In some embodiments, the subject weighs≥5 kg to <15 kg and the IL-4R antagonist is administered at a dose of about 200 mg Q4W.

In some embodiments, the subject weighs≥15 kg to <30 kg. In some embodiments, the subject weighs≥15 kg to <30 kg and the IL-4R antagonist is administered at a dose of about 200 mg Q2W. In some embodiments, the subject weighs≥15 kg to <30 kg and the IL-4R antagonist is administered at a dose of about 300 mg Q4W.

In some embodiments, the subject weighs≥30 kg to <60 kg. In some embodiments, the subject weighs≥30 kg to <60 kg and the IL-4R antagonist is administered at a dose of about 300 mg Q2W. In some embodiments, the subject weighs≥30 kg to <60 kg and the IL-4R antagonist is administered at a dose of about 200 mg Q2W.

In some embodiments, the subject weighs 60 kg. In some embodiments, the subject weighs≥60 kg and the IL-4R antagonist is administered at a dose of about 300 mg QW. In some embodiments, the subject weighs 60 kg and the IL-4R antagonist is administered at a dose of about 300 mg Q2W.

In some embodiments, the subject weighs≥40 kg. In some embodiments, the subject weighs≥40 kg and the IL-4R antagonist is administered at a dose of about 300 mg QW.

In some embodiments, the IL-4R antagonist is administered in combination with a second therapeutic agent or therapy. In some embodiments, the second therapeutic agent or therapy is an IL-1β inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a topical corticosteroid, an oral corticosteroid, a systemic corticosteroid, an inhaled corticosteroid, a glucocorticoid, a PPI, a decongestant, an antihistamine, a non-steroidal anti-inflammatory drug (NSAID), esophagus dilation, a feeding tube, allergen removal, or diet management. In some embodiments, the second therapeutic agent or therapy is diet management. In some embodiments, the second therapeutic agent or therapy is a PPI.

In some embodiments, treatment with the IL-4R antagonist for at least 16 weeks results in:
a decrease in peak esophageal intraepithelial eosinophil count;
a reduction in the severity and/or extent of disease as measured by Eosinophilic Esophagitis-Histology Scoring System (EoE-HSS);
an improvement in esophageal anatomical characteristics as measured by Eosinophilic Esophagitis-Endoscopic Reference Score (EoE-EREFS);
an increase in body weight for age percentile; and/or
a decrease in a normalized enrichment score (NES) for a type 2 inflammation panel and/or an EoE diagnostic panel of genes.

In some embodiments, treatment with the IL-4R antagonist for 16 weeks results in a peak esophageal intraepithelial eosinophil count of ≥6 eos/hpf, and/or a decrease in peak esophageal intraepithelial eosinophil count of at least 75%.

In some embodiments, the IL-4R antagonist is contained in a container selected from the group consisting of a glass vial, a syringe, a pre-filled syringe, a pen delivery device, and an autoinjector. In some embodiments, the IL-4R antagonist is contained in a pre-filled syringe. In some embodiments, the pre-filled syringe is a single-dose pre-filled syringe. In some embodiments, the IL-4R antagonist is contained in an autoinjector. In some embodiments, the IL-4R antagonist is contained in a pen delivery device.

In another aspect, the present disclosure provides pharmaceutical compositions comprising an IL-4R antagonist for use in treating, preventing, or ameliorating at least one symptom of EoE in a pediatric subject<12 years of age (e.g., a subject≥1 and <12 years old) or in a pediatric or adolescent subject having a body weight<40 kg at baseline (e.g., having a body weight≥5 kg and <40 kg at baseline). In some embodiments, the IL-4R antagonist is an anti-IL-4R antibody, or an antigen-binding fragment thereof, that comprises an HCDR1 comprising or consisting of the amino acid sequence of SEQ ID NO:3, an HCDR2 comprising or consisting of the amino acid sequence of SEQ ID NO:4, an HCDR3 comprising or consisting of the amino acid sequence of SEQ ID NO:5, an LCDR1 comprising or consisting of the amino acid sequence of SEQ ID NO:6, an LCDR2 comprising or consisting of the amino acid sequence of LGS, and an LCDR3 comprising or consisting of the amino acid sequence of SEQ ID NO:8. In some embodiments, the anti-IL-4R antibody or antigen-binding fragment thereof comprises an HCVR comprising the amino acid sequence of SEQ ID NO:1 and an LCVR comprising the amino acid sequence of SEQ ID NO:2. In some embodiments, the anti-IL-4R antibody comprises a heavy chain comprising the amino acid sequence of SEQ ID NO:9 and a light chain comprising the amino acid sequence of SEQ ID NO:10. In some embodiments, the IL-4R antagonist is dupilumab. In some embodiments, the pharmaceutical composition is formulated to administer about 50 mg to about 600 mg of the IL-4R antagonist in each dose, e.g., about 100 mg, about 200 mg, or about 300 mg.

In another aspect, the present disclosure provides for the use of an IL-4R antagonist in the preparation of a medicament for treating, preventing, or ameliorating at least one symptom of EoE in a pediatric subject<12 years of age (e.g., a subject≥1 and <12 years old) or in a pediatric or adolescent subject having a body weight<40 kg at baseline (e.g., having a body weight≥5 kg and <40 kg at baseline). In some embodiments, the IL-4R antagonist is an anti-IL-4R antibody, or an antigen-binding fragment thereof, that comprises an HCDR1 comprising or consisting of the amino acid sequence of SEQ ID NO:3, an HCDR2 comprising or consisting of the amino acid sequence of SEQ ID NO:4, an HCDR3 comprising or consisting of the amino acid sequence of SEQ ID NO:5, an LCDR1 comprising or consisting of the amino acid sequence of SEQ ID NO:6, an LCDR2 comprising or consisting of the amino acid sequence of LGS, and an LCDR3 comprising or consisting of the amino acid sequence of SEQ ID NO:8. In some embodiments, the anti-IL-4R antibody or antigen-binding fragment thereof comprises an HCVR comprising the amino acid sequence of SEQ ID NO:1 and an LCVR comprising the amino acid sequence of SEQ ID NO:2. In some embodiments, the anti-IL-4R antibody comprises a heavy chain comprising the amino acid sequence of SEQ ID NO:9 and a light chain comprising the amino acid sequence of SEQ ID NO:10. In some embodiments, the IL-4R antagonist is dupilumab. In some embodiments, the medicament is formulated to administer about 50 mg to about 600 mg of the IL-4R antagonist in each dose, e.g., about 100 mg, about 200 mg, or about 300 mg.

In another aspect, the present disclosure provides therapeutic dosage forms of a pharmaceutical composition comprising an IL-4R antagonist for use in treating, preventing, or ameliorating at least one symptom of EoE in a pediatric subject<12 years of age (e.g., a subject≥1 and <12 years old) or in a pediatric or adolescent subject having a body weight<40 kg at baseline (e.g., having a body weight≥5 kg and <40 kg at baseline). In some embodiments, the IL-4R antagonist is an anti-IL-4R antibody, or an antigen-binding fragment thereof, that comprises an HCDR1 comprising or consisting of the amino acid sequence of SEQ ID NO:3, an HCDR2 comprising or consisting of the amino acid sequence of SEQ ID NO:4, an HCDR3 comprising or consisting of the amino acid sequence of SEQ ID NO:5, an LCDR1 comprising or consisting of the amino acid sequence of SEQ ID NO:6, an LCDR2 comprising or consisting of the amino acid sequence of LGS, and an LCDR3 comprising or consisting of the amino acid sequence of SEQ ID NO:8. In some embodiments, the anti-IL-4R antibody or antigen-binding fragment thereof comprises an HCVR comprising the amino acid sequence of SEQ ID NO:1 and an LCVR comprising the amino acid sequence of SEQ ID NO:2. In some embodiments, the anti-IL-4R antibody comprises a heavy chain comprising the amino acid sequence of SEQ ID NO:9 and a light chain comprising the amino acid sequence of SEQ ID NO:10. In some embodiments, the IL-4R antagonist is dupilumab. In some embodiments, the therapeutic dosage form is formulated to administer about 50 mg to about 600 mg of the IL-4R antagonist in each dose, e.g., about 100 mg, about 200 mg, or about 300 mg.

In another aspect, the present disclosure provides kits comprising an IL-4R antagonist, pharmaceutical composition comprising an IL-4R antagonist, or medicament comprising an IL-4R antagonist, for use in treating, preventing, or ameliorating at least one symptom of EoE in a pediatric subject<12 years of age (e.g., a subject≥1 and <12 years old) or in a pediatric or adolescent subject having a body weight<40 kg at baseline (e.g., having a body weight 5 kg and <40 kg at baseline). In some embodiments, the kit also comprises a label or instructions for use.

Other embodiments will be apparent from a review of the ensuing detailed description.

### DETAILED DESCRIPTION

### Definitions

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

“Eosinophilic esophagitis” or “EoE,” refers to an inflammatory disease characterized by abnormal eosinophilic inflammation within the esophagus and esophageal dysfunction. The primary symptoms of EoE include, but are not limited to, chest and abdominal pain, dysphagia, heartburn, food refusal, vomiting, food impaction, and a failure to thrive. Histopathological features of EoE are related to the inflammation in the esophagus and may include fixed or transient concentric rings, longitudinal furrows, white plaques, reduced mucosal vascularity, fragile or crepe-like mucosa, and strictures. Furrows and white plaques are more common in children, while rings are more common in adults than in children. EoE is typically diagnosed by endoscopy of the esophagus with biopsy followed by microscopic and biochemical analysis of the esophageal mucosal lining. The current consensus recommendation for diagnosis of EoE is peak esophageal intraepithelial eosinophil cell count≥15 eos/hpf.

As used herein, the term “subject in need thereof” refers to a human or a non-human animal that exhibits one or more symptoms or indications of eosinophilic esophagitis, and/or who has been diagnosed with eosinophilic esophagitis. In certain embodiments, the term includes subjects that show elevated levels of one or more EoE-associated biomarkers (described elsewhere herein) and/or subjects having a gene expression profile that is associated with EoE (“EoE disease transcriptome”). For example, in some embodiments a subject to be treated according to the methods of the disclosure is a subject with elevated levels of IgE, serum TARC, and/or eotaxin-3, a subject having a gene expression profile consistent with the published EoE gene expression signature (Dellon, et al., Clin Transl Gastroenterol, 2017, 8(2):e74), or a subject having an altered expression level of one or more genes of the published EoE gene expression signature. As used herein, the terms “subject” and “patient” are used interchangeably.

The term “subject in need thereof” may also include, e.g., subjects who, prior to treatment, exhibit (or have exhibited) one or more indications of EoE such as, e.g., esophageal overexpression of pro-inflammatory mediators such as mast cells, eosinophilic infiltration of the esophagus, thickening of the esophageal wall, dysphagia, food impaction, and chest and abdominal pain and/or an elevated level of a EoE-associated biomarker. The term also includes subjects with elevated peripheral eosinophil counts (e.g., ≥100, ≥150, ≥200, or ≥300 cells/μL) or elevated serum IgE (e.g., >150 kU/L).

The term “eosinophilic infiltration” refers to the presence of eosinophils in an organ or tissue including blood, esophagus, stomach, duodenum, and ileum of a subject. In the context of the present disclosure, the term “eosinophilic infiltration” refers to presence of eosinophils in the mucosal lining of a region of the gastro-intestinal tract including, but not limited to, esophagus and stomach. Eosinophilic infiltration is analyzed, for example, in an esophageal tissue biopsy of a subject having EoE. According to some embodiments, “eosinophilic infiltration” refers to the presence of ≥15 eosinophils per high power field in the esophagus, or in two or more of the proximal, mid, and distal regions of the esophagus. The term “high power field” refers to a standard total magnification of 400× by a microscope used to view eosinophils in a tissue, e.g., from the esophagus of a subject. Thus, in some embodiments, a “subject in need thereof” refers to a subject who shows the presence of ≥15 eosinophils (“eos”) per high power field (“hpf”) in the esophagus, e.g., in two or more of the proximal, mid, and distal regions of the esophagus. In certain embodiments, “eosinophilic infiltration” includes infiltration into a tissue by leukocytes, for example, lymphocytes, neutrophils and mast cells. The leukocyte infiltration into, e.g., esophageal tissue can be detected by cell surface markers such as eosinophil-specific markers (e.g., CD11c^{Low/Neg}, SiglecF⁺, F4/80⁺, EMR1⁺, Siglec 8⁺, and MBP2⁺), macrophage-specific markers (e.g., CD11b⁺, F4/80⁺, CD14⁺, EMR1⁺, and CD68⁺), neutrophil-specific markers (e.g., CD11b⁺, Ly6G⁺, Ly6C⁺, CD11b⁺, and CD66b⁺), and T-cell-specific markers (e.g., CD3⁺, CD4⁺, and CD8⁺).

In some embodiments, a subject is unresponsive, inadequately responsive, or intolerant to treatment with a standard of care treatment (e.g., swallowed topical corticosteroid or PPI). As used herein, a subject who is “unresponsive” to treatment is a subject whose disease symptoms do not improve, do not significantly improve, or worsen upon treatment with the standard of care for a defined period of time, e.g., as measured using an EoE-related parameter as disclosed herein (e.g., as measured by esophageal intraepithelial eosinophil count, change in the severity and/or extent of histologic features in the esophagus, or change in one or more esophageal characteristics as measured by EoE-EREFS). As used herein, a subject who is “inadequately responsive” to treatment is a subject whose disease symptoms may partially improve upon treatment with the standard of care but do not improve to a level indicative of disease control, or whose disease symptoms initially improve but do not achieve stable long-term disease control, e.g., as measured using an EoE-related parameter as disclosed herein (e.g., as measured by esophageal intraepithelial eosinophil count, change in the severity and/or extent of histologic features in the esophagus, or change in one or more esophageal characteristics as measured by EoE-EREFS). As used herein, a subject who is “intolerant” to treatment is a subject who is unable to tolerate the adverse effects of the treatment, e.g., at a therapeutic dose or a subtherapeutic dose.

Although any methods and materials similar or equivalent to those described herein can be used in the practice of the disclosure, the typical methods and materials are now described. All publications mentioned herein are incorporated herein by reference in their entirety.

### Therapeutic Methods

In one aspect, methods for treating, preventing, or ameliorating one or more symptoms of eosinophilic esophagitis (EoE) in a subject are provided. In some embodiments, the subject is a pediatric or adolescent subject<18 years of age. In some embodiments, the subject is a pediatric subject<12 years of age. In some embodiments, the subject is ≥1 year to <12 years of age. In some embodiments, the subject is ≥1 and <4 years of age. In some embodiments, the subject is ≥1 and <5 years of age. In some embodiments, the subject is ≥2 and <8 years of age. In some embodiments, the subject is ≥5 and <12 years of age. In some embodiments, the subject is ≥8 and <12 years of age.

In some embodiments, a subject to be treated according to the methods disclosed herein has a history of documented diagnosis of EoE by endoscopic biopsy, as demonstrated by intraepithelial eosinophilic infiltration from at least 1 esophageal region. In some embodiments, the subject has a baseline peak eosinophil count 15 eos/hpf. “Peak esophageal intraepithelial eosinophil count,” as used herein, refers to the number of eosinophils contained within one high power field (hpf), e.g., at 400×. In some embodiments, the subject has an intraepithelial eosinophilic infiltration peak cell count≥15 eos/hpf as measured by endoscopic biopsy in at least two of, or in all three of, the proximal esophageal region, mid esophageal region, and distal esophageal region. In some embodiments, the subject has a mean peak esophageal intraepithelial eosinophil count of ≥20 eos/hpf, ≥25 eos/hpf, ≥30 eos/hpf, ≥35 eos/hpf, ≥40 eos/hpf, ≥45 eos/hpf, ≥50 eos/hpf, ≥55 eos/hpf, ≥60 eos/hpf, ≥65 eos/hpf, ≥70 eos/hpf, ≥75 eos/hpf, ≥80 eos/hpf, ≥85 eos/hpf, or ≥90 eos/hpf.

In some embodiments, a subject to be treated according to the methods disclosed herein has a history of one or more symptoms of EoE, such as but not limited to, abdominal pain, chest pain, acid reflux, food regurgitation, dysphagia, vomiting, food impaction, feeding difficulties, refusal to eat, or failure to thrive. In some embodiments, the subject has a history of one or more symptoms of EoE for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 50 weeks, or longer. In some embodiments, the subject has a history of one or more symptoms of EoE for at least 6 months, at least 1 year, at least 2 years, or longer. In some embodiments, the subject has a body weight that is below average for their age percentile.

In some embodiments, a subject to be treated according to the methods disclosed herein has a history of prior treatment with one or more standard-of-care therapies for EoE, such as but not limited to dietary modification (e.g., food-elimination diets), swallowed topical corticosteroids, glucocorticoids, proton pump inhibitor (PPI) therapy (e.g., high-dose PPI regimens), or esophageal dilation. In some embodiments, a subject to be treated is a subject who is non-responsive, inadequately responsive, intolerant, or resistant to one or more of the current standard-of-care therapies for EoE. In some embodiments, a subject to be treated has a contraindication for one or more standard-of-care therapies.

In some embodiments, a subject to be treated has a body weight≥5 kg at baseline. In some embodiments, a subject to be treated has a body weight<60 kg at baseline. In some embodiments, a subject to be treated has a body weight≥5 kg and <60 kg at baseline. In some embodiments, a subject to be treated has a body weight≥5 kg and <15 kg at baseline. In some embodiments, a subject to be treated has a body weight≥15 kg and <30 kg at baseline. In some embodiments, a subject to be treated has a body weight≥30 kg and <60 kg at baseline.

In some embodiments, a subject to be treated is a pediatric or adolescent patient having a body weight<40 kg at baseline. In some embodiments, a subject to be treated has a body weight≥5 kg and <40 kg at baseline.

In some embodiments, a subject to be treated has an altered level of one or more biomarkers of EoE or has a gene signature profile for EoE-associated genes that is indicative of or consistent with the published gene signature profile for EoE patients. EoE-associated biomarkers and gene expression panels that are diagnostic for EoE are described in the art, e.g., in Sherrill, et al., Genes Immun, 2014, 15(6):361-369; Dellon, et al., Clin Transl Gastroenterol, 2017, 8(2):e74; and US Patent Publication No. 2017/0067111. In some embodiments, the subject has an elevated level of eotaxin-3, serum TARC, and/or total IgE. In some embodiments, a subject to be treated has an elevated level of one or more EoE-associated genes such as TNFAIP6, LRRC31, SLC26A4-AS1, ALOX15, CCL26, TGM6, NRXN1, PMCH, SLC26A4, CXCL1, CCR3, TREML2, POSTN, LURAP1L, or CXCL6. In some embodiments, a subject to be treated has a reduced level of one or more EoE-associated genes such as CRTAC1, BC107108, SFTA2, C2orf16, KRTAP3-2, PLNIPRP3, CIDEA, FLG, SLC8A1-AS1, SPINK5, SPINK7, SPINK8, DPCR1, MUC22, CRISP2, DSG1, GYS2, or CRISP3.

In some embodiments, a subject to be treated has, or has had, at least one comorbidity. In some embodiments, the comorbidity is asthma, atopic dermatitis, hand and food eczema, allergic rhinitis, oral allergy syndrome, or food allergy (e.g., peanut allergy).

In some embodiments, a subject to be treated has, or has had, a concomitant type 2 inflammatory condition. As used herein, a “type 2 inflammation condition” is a disease, disorder, or condition associated with a T helper 2 (T_{H}2)-mediated immune response (Gandhi, et al., Nat Rev Drug Discov., 2016, 15(1):35-50). Non-limiting examples of type 2 inflammatory conditions include asthma, chronic rhinosinusitis, allergic rhinitis, allergic fungal rhinosinusitis, chronic sinusitis, allergic bronchopulmonary aspergillosis (ABPA), unified airway disease, eosinophilic granulomatosis with polyangiitis (EGPA, formerly known as Churg-Strauss syndrome), gastroesophageal reflux disease (GERD), atopic conjunctivitis, atopic dermatitis, vasculitis, cystic fibrosis (CF), chronic obstructive pulmonary disease (COPD), chronic rhinosinusitis with nasal polyps (CRSwNP), aspirin hypersensitivity, non-steroidal anti-inflammatory drug (NSAID) hypersensitivity (e.g., NSAIDs Exacerbated Respiratory Disease, or NSAID-ERD), perennial allergic rhinitis (PAR), chronic eosinophilic pneumonia (CEP) and exercise induced bronchospasm. In some embodiments, the subject has a concomitant atopic disease or condition selected from the group consisting of food allergy, atopic dermatitis, asthma, chronic rhinosinusitis, allergic rhinitis, or allergic conjunctivitis.

In some embodiments, a subject to be treated is a subject who is susceptible to an allergen, e.g., a subject having a food allergy or oral allergy syndrome. For example, in some embodiments, the subject may exhibit one of the following characteristics: (a) is prone to allergic reactions or responses when exposed to one or more allergens; (b) has previously exhibited an allergic response or reaction to one or more allergens; (c) has a known history of allergies; and/or (d) exhibits a sign or symptom of an allergic response or anaphylaxis. As used herein, the phrases “allergic response,” “allergic reaction,” “allergic symptom,” and the like, include one or more signs or symptoms selected from the group consisting of urticaria (e.g., hives), angioedema, rhinitis, asthma, vomiting, sneezing, runny nose, sinus inflammation, watery eyes, wheezing, bronchospasm, reduced peak expiratory flow (PEF), gastrointestinal distress, flushing, swollen lips, swollen tongue, reduced blood pressure, anaphylaxis, and organ dysfunction/failure. An “allergic response,” “allergic reaction,” “allergic symptom,” etc., also includes immunological responses and reactions such as, e.g., increased IgE production, increased allergen-specific immunoglobulin production and/or eosinophilia. In certain embodiments, the subject is allergic to an allergen associated with EoE or that renders the subject susceptible and/or prone to developing EoE. In some embodiments, the allergen is contained within or derived from a food item such as, e.g., dairy products (e.g., cow's milk), egg, wheat, soy, corn, rye, fish, shellfish, peanuts, tree nuts. In some embodiments, the allergen is contained within or derived from a non-food item such as, e.g., dust (e.g., containing dust mite), pollen, insect venom (e.g., venom of bees, wasps, mosquitoes, etc.), mold, animal dander, latex, medication, drugs, ragweed, grass, or birch.

In some embodiments, a subject to be treated is selected on the basis of exhibiting one or more inclusion criteria disclosed in Example 1. In some embodiments, a subject to be treated is further selected on the basis of not exhibiting one or more exclusion criteria disclosed in Example 1.

### Interleukin-4 Receptor Antagonists

In some embodiments, the methods of the present disclosure comprise administering to a subject in need thereof (e.g., a subject having EoE who is <12 years of age, or a subject having EoE who is ≥1 year and <12 years of age) an interleukin-4 receptor (IL-4R) antagonist or a pharmaceutical composition comprising an IL-4R antagonist. As used herein, an “IL-4R antagonist” (also referred to herein as an “IL-4R inhibitor”, an “IL-4R blocker,” or an “IL-4Rα antagonist”) is any agent that binds to or interacts with IL-4Rα or an IL-4R ligand, and inhibits or attenuates the normal biological signaling function of a type 1 and/or a type 2 IL-4 receptor. Human IL-4Rα has the amino acid sequence of SEQ ID NO:11. A type 1 IL-4 receptor is a dimeric receptor comprising an IL-4Rα chain and a γc chain. A type 2 IL-4 receptor is a dimeric receptor comprising an IL-4Rα chain and an IL-13Rα1 chain. Type 1 IL-4 receptors interact with and are stimulated by IL-4, while type 2 IL-4 receptors interact with and are stimulated by both IL-4 and IL-13. Thus, the IL-4R antagonists that can be used in the methods of the present disclosure may function by blocking IL-4-mediated signaling, IL-13-mediated signaling, or both IL-4- and IL-13-mediated signaling. The IL-4R antagonists of the present disclosure may thus prevent the interaction of IL-4 and/or IL-13 with a type 1 or type 2 receptor.

Non-limiting examples of categories of IL-4R antagonists include small molecule IL-4R inhibitors, anti-IL-4R aptamers, peptide-based IL-4R inhibitors (e.g., “peptibody” molecules), “receptor-bodies” (e.g., engineered molecules comprising the ligand-binding domain of an IL-4R component), and antibodies or antigen-binding fragments of antibodies that specifically bind human IL-4Ra. As used herein, IL-4R antagonists also include antigen-binding proteins that specifically bind IL-4 and/or IL-13.

### Anti-IL-4Rα Antibodies and Antigen-Binding Fragments Thereof

In certain exemplary embodiments of the present disclosure, the IL-4R antagonist is an anti-IL-4Rα antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the anti-IL-4R antibody (or antigen-binding portion thereof) are identical to the human germline sequences. In some embodiments, one or more FRs of the anti-IL-4R antibody (or antigen-binding portion thereof) are naturally or artificially modified.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add, or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed by the term “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2, (x) V_{L}-C_{H}3; (Xi) V_{L}-C_{H}1-C_{H}2; (Xii) V_{L}-C_{H}1-C_{H}2-C_{H}3; (Xiii) V_{L}-C_{H}2-C_{H}3; and (Xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The constant region of an antibody is important in the ability of an antibody to fix complement and mediate cell-dependent cytotoxicity. Thus, in some embodiments the isotype of an antibody may be selected on the basis of whether it is desirable for the antibody to mediate cytotoxicity.

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, in some embodiments the methods of the present disclosure comprise the use of bispecific antibodies wherein one arm of an immunoglobulin is specific for IL-4Rα or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein, et al. 2012, mAbs, 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane, et al., J. Am. Chem. Soc., [Epub: Dec. 4, 2012]).

In some embodiments, the antibodies used in the methods of the present disclosure are human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor, et al., (1992) Nucl. Acids Res., 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

According to certain embodiments, the antibodies used in the methods of the present disclosure specifically bind IL-4Rα. The term “specifically binds,” as used herein, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. In some embodiments, an antibody that “specifically binds” IL-4Rα binds to IL-4Rα or a portion thereof with an equilibrium dissociation constant (K_{D}) of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 0.5 nM, less than about 0.25 nM, less than about 0.1 nM or less than about 0.05 nM, as measured in a surface plasmon resonance assay (e.g., BIAcore™, Biacore Life Sciences division of GE Healthcare, Piscataway, NJ). In some embodiments, an antibody that specifically binds to a target antigen (e.g., IL-4Rα) can also specifically bind to another antigen, e.g., an ortholog of the target antigen. For example, in some embodiments, an isolated antibody that specifically binds human IL-4Rα exhibits cross-reactivity to other antigens, such as IL-4Rα molecules from other (non-human) species.

In some embodiments, the IL-4R antagonist is an anti-IL-4Rα antibody, or antigen-binding fragment thereof, comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693, incorporated by reference herein. In some embodiments, the IL-4R antagonist is an anti-IL-4Rα antibody or antigen-binding fragment thereof that comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:2. In some embodiments, the IL-4R antagonist is an anti-IL-4Rα antibody or antigen-binding fragment thereof that comprises three HCDRs (HCDR1, HCDR2, and HCDR3) and three LCDRs (LCDR1, LCDR2, and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence of LGS, and the LCDR3 comprises the amino acid sequence of SEQ ID NO:8.

In some embodiments, the anti-IL-4R antibody or antigen-binding fragment thereof comprises the HCDR1, HCDR2, HCDR3, LCDR1, LCDR2, and LCDR3 of SEQ ID NOs:3, 4, 5, 6, 7, and 8, respectively, and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:1 and an LCVR having at least 85% sequence identity (e.g., at least 90%, 91%, 92%, 93%, 94%, 95%, 96%, 97%, 98%, or 99% sequence identity) to the amino acid sequence of SEQ ID NO:2. In some embodiments, the anti-IL-4R antibody or antigen-binding fragment thereof comprises an HCVR comprising SEQ ID NO:1 and an LCVR comprising SEQ ID NO:2.

In some embodiments, the anti-IL-4R antibody comprises a heavy chain comprising the amino acid sequence of SEQ ID NO:9. In some embodiments, the anti-IL-4R antibody comprises a light chain comprising the amino acid sequence of SEQ ID NO:10.

An exemplary antibody comprising a heavy chain comprising the amino acid sequence of SEQ ID NO:9 and a light chain comprising the amino acid sequence of SEQ ID NO:10 is the fully human anti-IL-4R antibody known as dupilumab. According to certain exemplary embodiments, the methods of the present disclosure comprise the use of dupilumab. As used herein, “dupilumab” also includes bioequivalents of dupilumab. The term “bioequivalent,” as used herein with reference to dupilumab, refers to anti-IL-4R antibodies or IL-4R-binding proteins or fragments thereof that are pharmaceutical equivalents or pharmaceutical alternatives whose rate and/or extent of absorption do not show a significant difference with that of dupilumab when administered at the same molar dose under similar experimental conditions, either single dose or multiple dose. In some embodiments, the term refers to antigen-binding proteins that bind to IL-4R which do not have clinically meaningful differences with dupilumab in their safety, purity and/or potency.

Other anti-IL-4Rα antibodies that can be used in the context of the methods of the present disclosure include, e.g., the antibody referred to and known in the art as AMG317 (Corren, et al., 2010, Am J Respir Crit Care Med., 181(8):788-796), or MEDI 9314, or any of the anti-IL-4Rα antibodies as set forth in U.S. Pat. Nos. 7,186,809, 7,605,237, 7,638,606, 8,092,804, 8,679,487, 8,877,189, 10,774,141, or International Patent Publication Nos. WO2020/096381, WO 2020/182197, WO2020/239134, WO 2021/213329, WO2022/052974, WO2022/136669, or WO2022/136675, the contents of each of which are incorporated by reference herein.

In some embodiments, an anti-IL-4Rα antibody or antigen-binding fragment thereof for use in the methods of the present disclosure comprises one or more CDR, HCVR, and/or LCVR sequences set forth in Table 7 below.

In some embodiments, an anti-IL-4Rα antibody comprises (i) an HCVR comprising the amino acid sequence of SEQ ID NO:32 (SCB-VH-59), SEQ ID NO:33 (SCB-VH-60), SEQ ID NO:34 (SCB-VH-61), SEQ ID NO:35 (SCB-VH-62), SEQ ID NO:36 (SCB-VH-63), SEQ ID NO:37 (SCB-VH-64), SEQ ID NO:38 (SCB-VH-65), SEQ ID NO:39 (SCB-VH-66), SEQ ID NO:40 (SCB-VH-67), SEQ ID NO:41 (SCB-VH-68), SEQ ID NO:42 (SCB-VH-69), SEQ ID NO:43 (SCB-VH-70), SEQ ID NO:44 (SCB-VH-71), SEQ ID NO:45 (SCB-VH-72), SEQ ID NO:46 (SCB-VH-73), SEQ ID NO:47 (SCB-VH-74), SEQ ID NO:48 (SCB-VH-75), SEQ ID NO:49 (SCB-VH-76), SEQ ID NO:50 (SCB-VH-77), SEQ ID NO:51 (SCB-VH-78), SEQ ID NO:52 (SCB-VH-79), SEQ ID NO:53 (SCB-VH-80), SEQ ID NO:54 (SCB-VH-81), SEQ ID NO:55 (SCB-VH-82), SEQ ID NO:56 (SCB-VH-83), SEQ ID NO:57 (SCB-VH-84), SEQ ID NO:58 (SCB-VH-85), SEQ ID NO:59 (SCB-VH-86), SEQ ID NO:60 (SCB-VH-87), SEQ ID NO:61 (SCB-VH-88), SEQ ID NO:62 (SCB-VH-89), SEQ ID NO:63 (SCB-VH-90), SEQ ID NO:64 (SCB-VH-91), SEQ ID NO:65 (SCB-VH-92), or SEQ ID NO:66 (SCB-VH-93); and (ii) an LCVR comprising the amino acid sequence of SEQ ID NO:12 (SCB-VL-39), SEQ ID NO:13 (SCB-VL-40), SEQ ID NO:14 (SCB-VL-41), SEQ ID NO:15 (SCB-VL-42), SEQ ID NO:16 (SCB-VL-43), SEQ ID NO:17 (SCB-VL-44), SEQ ID NO:18 (SCB-VL-45), SEQ ID NO:19 (SCB-VL-46), SEQ ID NO:20 (SCB-VL-47), SEQ ID NO:21 (SCB-VL-48), SEQ ID NO:22 (SCB-VL-49), SEQ ID NO:23 (SCB-VL-50), SEQ ID NO:24 (SCB-VL-51), SEQ ID NO:25 (SCB-VL-52), SEQ ID NO:26 (SCB-VL-53), SEQ ID NO:27 (SCB-VL-54), SEQ ID NO:28 (SCB-VL-55), SEQ ID NO:29 (SCB-VL-56), SEQ ID NO:30 (SCB-VL-57), or SEQ ID NO:31 (SCB-VL-58). In some embodiments, the anti-IL-4Rα antibody comprises an HCVR comprising the amino acid sequence of SEQ ID NO:64 (SCB-VH-91) and an LCVR comprising the amino acid sequence of SEQ ID NO:17 (SCB-VL-44), SEQ ID NO:27 (SCB-VL-54), or SEQ ID NO:28 (SCB-VL-55).

In some embodiments, an anti-IL-4Rα antibody comprises an amino acid sequence pair selected from the group consisting of: SEQ ID NOs:67/68 (MEDI-1-VH/MEDI-1-VL); SEQ ID NOs:69/70 (MEDI-2-VH/MEDI-2-VL); SEQ ID NOs:71/72 (MEDI-3-VH/MEDI-3-VL); SEQ ID NOs:73/74 (MEDI-4-VH/MEDI-4-VL); SEQ ID NOs:75/76 (MEDI-5-VH/MEDI-5-VL); SEQ ID NOs:77/78 (MEDI-6-VH/MEDI-6/VL); SEQ ID NOs:79/80 (MEDI-7-VH/MEDI-7-VL); SEQ ID NOs:81/82 (MEDI-8-VH/MEDI-8-VL); SEQ ID NOs:83/84 (MEDI-9-VH/MEDI-9-VL); SEQ ID NOs:85/86 (MEDI-10-VH/MEDI-10-VL); SEQ ID NOs:87/88 (MEDI-11-VH/MEDI-11/VL); SEQ ID NOs:89/90 (MEDI-12-VH/MEDI-12-VL); SEQ ID NOs:91/92 (MEDI-13-VH/MEDI-13-VL); SEQ ID NOs:93/94 (MEDI-14-VH/MEDI-14-VL); SEQ ID NOs:95/96 (MEDI-15-VH/MEDI-15-VL); SEQ ID NOs:97/98 (MEDI-16-VH/MEDI-16/VL); SEQ ID NOs:99/100 (MEDI-17-VH/MEDI-17-VL); SEQ ID NOs:101/102 (MEDI-18-VH/MEDI-18-VL); SEQ ID NOs:103/104 (MEDI-19-VH/MEDI-19-VL); SEQ ID NOs:105/106 (MEDI-20-VH/MEDI-20-VL); SEQ ID NOs:107/108 (MEDI-21-VH/MEDI-21-VL); SEQ ID NOs:109/110 (MEDI-22-VH/MEDI-22-VL); SEQ ID NOs:111/112 (MEDI-23-VH/MEDI-23-VL); SEQ ID NOs:113/114 (MEDI-24-VH/MEDI-24-VL); SEQ ID NOs:115/116 (MEDI-25-VH/MEDI-25-VL); SEQ ID NOs:117/118 (MEDI-26-VH/MEDI-26-VL); SEQ ID NOs:119/120 (MEDI-27-VH/MEDI-27-VL); SEQ ID NOs:121/122 (MEDI-28-VH/MEDI-28-VL); SEQ ID NOs:123/124 (MEDI-29-VH/MEDI-29-VL); SEQ ID NOs:125/126 (MEDI-30-VH/MEDI-30-VL); SEQ ID NOs:127/128 (MEDI-31-VH/MEDI-31-VL); SEQ ID NOs:129/130 (MEDI-32-VH/MEDI-32-VL); SEQ ID NOs:131/132 (MEDI-33-VH/MEDI-33-VL); SEQ ID NOs:133/134 (MEDI-34-VH/MEDI-34-VL); SEQ ID NOs:135/136 (MEDI-35-VH/MEDI-35-VL); SEQ ID NOs:137/138 (MEDI-36-VH/MEDI-36-VL); SEQ ID NOs:139/140 (MEDI-37-VH/MEDI-37-VL); SEQ ID NOs:141/142 (MEDI-38-VH/MEDI-38-VL); SEQ ID NOs:143/144 (MEDI-39-VH/MEDI-39-VL); SEQ ID NOs:145/146 (MEDI-40-VH/MEDI-40-VL); SEQ ID NOs:147/148 (MEDI-41-VH/MEDI-41-VL); SEQ ID NOs:149/150 (MEDI-42-VH/MEDI-42-VL); and SEQ ID NOs:151/152 (MEDI-37GL-VH/MEDI-37GL-VL).

In some embodiments, an anti-IL-4Rα antibody comprises (i) an HCVR comprising the amino acid sequence of SEQ ID NO:153 (AJOU-1-VH), SEQ ID NO:154 (AJOU-2-VH), SEQ ID NO:155 (AJOU-3-VH), SEQ ID NO:156 (AJOU-4-VH), SEQ ID NO:157 (AJOU-5-VH), SEQ ID NO:158 (AJOU-6-VH), SEQ ID NO:159 (AJOU-7-VH), SEQ ID NO:160 (AJOU-8-VH), SEQ ID NO:161 (AJOU-9-VH), SEQ ID NO:162 (AJOU-10-VH), SEQ ID NO:163 (AJOU-69-VH), SEQ ID NO:164 (AJOU-70-VH), SEQ ID NO:165 (AJOU-71-VH), SEQ ID NO:166 (AJOU-72-VH), or SEQ ID NO:167 (AJOU-83-VH); and (ii) an LCVR comprising the amino acid sequence of SEQ ID NO:168 (AJOU-33-VL), SEQ ID NO:169 (AJOU-34-VL), SEQ ID NO:170 (AJOU-35-VL), SEQ ID NO:171 (AJOU-36-VL), SEQ ID NO:172 (AJOU-37-VL), SEQ ID NO:173 (AJOU-38-VL), SEQ ID NO:174 (AJOU-39-VL), SEQ ID NO:175 (AJOU-40-VL), SEQ ID NO:176 (AJOU-41-VL), SEQ ID NO:177 (AJOU-42-VL), SEQ ID NO:178 (AJOU-77-VL), SEQ ID NO:179 (AJOU-78-VL), SEQ ID NO:180 (AJOU-79-VL), SEQ ID NO:181 (AJOU-80-VL), SEQ ID NO:182 (AJOU-86-VL), SEQ ID NO:183 (AJOU-87-VL), SEQ ID NO:184 (AJOU-88-VL), SEQ ID NO:185 (AJOU-89-VL), SEQ ID NO:186 (AJOU-90-VL), or SEQ ID NO:187 (AJOU-91-VL).

In some embodiments, an anti-IL-4Rα antibody comprises (i) an HCVR comprising the amino acid sequence of SEQ ID NO:188 (REGN-VH-3), SEQ ID NO:189 (REGN-VH-19), SEQ ID NO:190 (REGN-VH-35), SEQ ID NO:191 (REGN-VH-51), SEQ ID NO:192 (REGN-VH-67), SEQ ID NO:193 (REGN-VH-83), SEQ ID NO:194 (REGN-VH-99), SEQ ID NO:195 (REGN-VH-115), SEQ ID NO:196 (REGN-VH-147), or SEQ ID NO:197 (REGN-VH-163); and (ii) an LCVR comprising the amino acid sequence of SEQ ID NO:198 (REGN-VL-11), SEQ ID NO:199 (REGN-VL-27), SEQ ID NO:200 (REGN-VL-43), SEQ ID NO:201 (REGN-VL-59), SEQ ID NO:202 (REGN-VL-75), SEQ ID NO:203 (REGN-VL-91), SEQ ID NO:204 (REGN-VL-107), SEQ ID NO:205 (REGN-VL-123), SEQ ID NO:206 (REGN-VL-155), or SEQ ID NO:207 (REGN-VL-171).

In some embodiments, an anti-IL-4Rα antibody comprises (i) an HCVR comprising the amino acid sequence of SEQ ID NO:208 (STSA-C27-VH), SEQ ID NO:209 (STSA-C27-6-33-VH), SEQ ID NO:210 (STSA-C27-7-33-VH), SEQ ID NO:211 (STSA-C27-24-56-VH), SEQ ID NO:212 (STSA-C27-47-56-VH), SEQ ID NO:213 (STSA-C27-33-33-VH), SEQ ID NO:214 (STSA-C27-56-56-VH), SEQ ID NO:215 (STSA-C27-78-78-VH), SEQ ID NO:216 (STSA-C27-82-58-VH), SEQ ID NO:217 (STSA-C27-54-54-VH), SEQ ID NO:218 (STSA-C27-36-36-VH), SEQ ID NO:219 (STSA-C27-53-53-VH), SEQ ID NO:220 (STSA-C27-67-67-VH), SEQ ID NO:221 (STSA-C27-55-55-VH), SEQ ID NO:222 (STSA-C27-59-59-VH), SEQ ID NO:223 (STSA-C27-58-58-VH), SEQ ID NO:224 (STSA-C27-52-52-VH), or SEQ ID NO:225 (STSA-C27-Y2-Y2-VH); and (ii) an LCVR comprising the amino acid sequence of SEQ ID NO:226 (STSA-C27-VL), SEQ ID NO:227 (STSA-C27-6-33-VL), SEQ ID NO:228 (STSA-C27-7-33-VL), SEQ ID NO:229 (STSA-C27-24-56-VL), SEQ ID NO:230 (STSA-C27-47-56-VL), SEQ ID NO:231 (STSA-C27-33-33-VL), SEQ ID NO:232 (STSA-C27-56-56-VL), SEQ ID NO:233 (STSA-C27-78-78-VL), SEQ ID NO:234 (STSA-C27-82-58-VL), SEQ ID NO:235 (STSA-C27-54-54-VL), SEQ ID NO:236 (STSA-C27-36-36-VL), SEQ ID NO:237 (STSA-C27-53-53-VL), SEQ ID NO:238 (STSA-C27-67-67-VL), SEQ ID NO:239 (STSA-C27-55-55-VL), SEQ ID NO:240 (STSA-C27-59-59-VL), SEQ ID NO:241 (STSA-C27-58-58-VL), SEQ ID NO:242 (STSA-C27-52-52-VL), or SEQ ID NO:243 (STSA-C27-Y2-Y2-VL).

In some embodiments, an anti-IL-4Rα antibody comprises (i) an HCVR comprising the amino acid sequence of SEQ ID NO:244 (Y0188-1 VH), SEQ ID NO:245 (Y0188-2 VH), SEQ ID NO:246 (Y0188-3 VH), SEQ ID NO:247 (Y0188-4 VH), SEQ ID NO:248 (Y0188-6 VH), SEQ ID NO:249 (Y0188-8 VH), SEQ ID NO:250 (Y0188-9 VH), SEQ ID NO:251 (Y0188-10 VH), SEQ ID NO:252 (Y0188-14 VH), SEQ ID NO:253 (HV3-15-14 VH), SEQ ID NO:254 (HV3-48-14 VH), SEQ ID NO:255 (HV3-73*2-14 VH), SEQ ID NO:256 (HV3-72-14 VH), SEQ ID NO:257 (Y01-14 VH), SEQ ID NO:258 (162-14 VH), or SEQ ID NO:259 (VH73-14 VH); and (ii) an LCVR comprising the amino acid sequence of SEQ ID NO:260 (Y0188-1 VL), SEQ ID NO:261 (Y0188-2 VL), SEQ ID NO:262 (Y0188-3 VL), SEQ ID NO:263 (Y0188-4 VL), SEQ ID NO:264 (Y0188-6 VL), SEQ ID NO:265 (Y0188-8 VL), SEQ ID NO:266 (Y0188-9 VL), SEQ ID NO:267 (Y0188-10 VL), SEQ ID NO:268 (Y0188-14 VL), SEQ ID NO:269 (Y01-14 VL), SEQ ID NO:270 (164-14 VL), SEQ ID NO:271 (KV4-14 VL), SEQ ID NO:272 (KV1-27-14 VL), SEQ ID NO:273 (KV1-9-14 VL), SEQ ID NO:274 (KV1-NL1-14 VL), or SEQ ID NO:275 (KV1 D-43-14 VL).

In some embodiments, an anti-IL-4Rα antibody used in the methods of the present disclosure can have pH-dependent binding characteristics. For example, an anti-IL-4Rα antibody for use as disclosed herein may exhibit reduced binding to IL-4Rα at acidic pH as compared to neutral pH. Alternatively, an anti-IL-4Rα antibody for use as disclosed herein may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to IL-4Rα at acidic pH as compared to neutral pH” is expressed in terms of a ratio of the K_{D} value of the antibody binding to IL-4Rα at acidic pH to the K_{D} value of the antibody binding to IL-4Rα at neutral pH (or vice versa). For example, an antibody or antigen-binding fragment thereof may be regarded as exhibiting “reduced binding to IL-4Rα at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Preparation of Human Antibodies

Methods for generating human antibodies in transgenic mice are known in the art. Any such known methods can be used in the context of the present disclosure to make human antibodies that specifically bind to human IL-4R.

Using VELOCIMMUNE™ technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

In one embodiment, a human antibody or antigen-binding fragment thereof that specifically binds IL-4R and that can be used in the methods disclosed herein comprises the three heavy chain CDRs (HCDR1, HCDR2, and HCDR3) contained within a heavy chain variable region (HCVR) having an amino acid sequence of SEQ ID NO:1, and the three light chain CDRs (LCVR1, LCVR2, and LCVR3) contained within a light chain variable region (LCVR) having an amino acid sequence of SEQ ID NO:2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani, et al., J. Mol. Biol., 273:927-948 (1997); and Martin, et al., Proc. Natl. Acad. Sci., USA 86:9268-9272 (1989). Public databases are also available for identifying CDR sequences within an antibody.

### Pharmaceutical Compositions and Kits

In one aspect, the present disclosure provides methods that comprise administering an IL-4R antagonist to a subject, wherein the IL-4R antagonist (e.g., an anti-IL-4R antibody) is contained within a pharmaceutical composition that comprises one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, the pharmaceutical composition comprises an injectable preparation, such as a dosage form for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of antibody administered to a subject according to the methods of the present disclosure may vary depending upon the age and the size of the subject, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, subject progress can be monitored by periodic assessment, and the dose adjusted accordingly. Moreover, interspecies scaling of dosages can be performed using well-known methods in the art (e.g., Mordenti, et al., 1991, Pharmaceut. Res., 8:1351). Specific exemplary doses of anti-IL-4R antibodies, and administration regimens involving the same, that can be used in the context of the present disclosure are disclosed elsewhere herein.

In some embodiments, an IL-4R antagonist or a pharmaceutical composition of the present disclosure is contained within a container. Thus, in another aspect, containers comprising an IL-4R antagonist or a pharmaceutical composition as disclosed herein are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. Typically, a reusable pen delivery device utilizes a replaceable cartridge that contains a pharmaceutical composition. Once the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™ OPTIPEN STARLET™, and OPTICLIK™ (sanofi-aventis, Frankfurt, Germany). Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science, 249:1527-1533. Other delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu, et al., 1987, J. Biol. Chem., 262:4429-4432).

In some embodiments, a pharmaceutical composition comprising an anti-IL-4R antibody is administered using a drug delivery device that is a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An exemplary sleeve-triggered auto-injector with manual needle insertion is described in International Publication WO2015/004052. Exemplary audible end-of-dose feedback mechanisms are described in International Publications WO2016/193346 and WO2016/193348. An exemplary needle-safety mechanism after using an auto-injector is described in International Publication WO2016/193352. An exemplary needle sheath remover mechanism for a syringe auto-injector is described in International Publication WO2016/193353. An exemplary support mechanism for supporting an axial position of a syringe is described in International Publication WO2016/193355.

In some embodiments, pharmaceutical compositions for use as described herein are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used in the context of the present disclosure are disclosed, e.g., in U.S. Pat. No. 8,945,559.

In another aspect, kits comprising an IL-4R antagonist or pharmaceutical composition as disclosed herein are provided. In some embodiments, the kit comprises an anti-IL-4R antibody, or a pharmaceutical composition comprising an anti-IL-4R antibody, and instructions for the use thereof in treating eosinophilic esophagitis in a pediatric subject<12 years of age (e.g., a subject≥1 and <12 years old) or in a pediatric or adolescent subject having a body weight<40 kg at baseline. In some embodiments, the instructions for use comprise administering the anti-IL-4R antibody or pharmaceutical composition in an amount, dosing frequency, and/or for a length of time as disclosed elsewhere herein.

### Dosage and Administration

In some embodiments, an IL-4R antagonist (e.g., anti-IL-4R antibody) is administered to a subject (e.g., a subject≥1 year to <12 years of age) according to the methods of the present disclosure in a therapeutically effective amount. As used herein with reference to an IL-4R antagonist, the phrase “therapeutically effective amount” means an amount of IL-4R antagonist that results in one or more of: (a) a reduction in the severity or duration of a symptom of eosinophilic esophagitis; (b) a reduction in the number of eosinophils in esophagus; (c) an improvement in one or more anatomical, endoscopic, or histological features of the esophagus; (d) increase in esophagus distensibility; (e) reduction in episodes of dysphagia or the intensity of dysphagia; (f) normalization of one or more EoE-associated biomarkers or gene expression signatures; and/or (g) a reduction in the use or need for concomitant or rescue treatment with another agent (e.g., reduced or eliminated use of systemic and/or swallowed topical corticosteroids, PPIs, etc.).

In the case of an anti-IL-4R antibody, a therapeutically effective amount can be from about 0.05 mg to about 600 mg, about 50 mg to about 600 mg, about 50 mg to about 300 mg, or about 100 mg to about 300 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-IL-4R antibody. In some embodiments, a therapeutically effective amount is from about 50 mg to about 600 mg, or from about 100 mg to about 600 mg, from about 50 mg to about 400 mg, or from about 100 mg to about 300 mg. In certain embodiments, 50 mg, 75 mg, 100 mg, 125 mg, 150 mg, 200 mg, 250 mg, or 300 mg of an anti-IL-4R antibody is administered to a subject.

The amount of IL-4R antagonist (e.g., anti-IL-4R antibody) contained within the individual doses may be expressed in terms of milligrams of antibody per kilogram of subject body weight (i.e., mg/kg). For example, the IL-4R antagonist may be administered to a subject at a dose of about 0.0001 to about 10 mg/kg of subject body weight, e.g., at a dose of about 1 mg/kg to about 10 mg/kg, at a dose of about 2 mg/kg to about 9 mg/kg, or at a dose of about 3 mg/kg to about 8 mg/kg. In some embodiments, the IL-4R antagonist may be administered to a subject at a dose of about 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

In some embodiments, the methods disclosed herein comprise administering an IL-4R antagonist to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, the methods disclosed herein comprise administering an IL-4R antagonist to a subject once weekly, once every two weeks, once every three weeks, or once every four weeks. In some embodiments, an IL-4R antagonist (e.g., an anti-IL-4R antibody as disclosed herein) is administered once weekly (QW), once every two weeks (Q2W), once every three weeks (Q3W), or once every four weeks (Q4W) in an amount of about 50 mg to about 600 mg, e.g., about 75 mg, 100 mg, 150 mg, 200 mg, or 300 mg.

In some embodiments, multiple doses of an IL-4R antagonist are administered to a subject over a defined time course. In some embodiments, the methods of the present disclosure comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks, or months). In some embodiments, the methods of the disclosure comprise sequentially administering to the patient a single initial dose of an IL-4R antagonist, followed by one or more secondary doses of the IL-4R antagonist, and optionally followed by one or more tertiary doses of the IL-4R antagonist.

The terms “initial dose,” “secondary doses,” and “tertiary doses,” refer to the temporal sequence of administration of the IL-4R antagonist. Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. The initial, secondary, and tertiary doses may all contain the same amount of IL-4R antagonist, but generally may differ from one another in terms of frequency of administration. In certain embodiments, however, the amount of IL-4R antagonist contained in the initial, secondary, and/or tertiary doses varies from one another (e.g., adjusted up or down as appropriate) during the course of treatment. In some embodiments, one or more (e.g., 1, 2, 3, 4, or 5) doses are administered at the beginning of the treatment regimen as “loading doses” followed by subsequent doses that are administered on a less frequent basis (e.g., “maintenance doses”). In some embodiments, the initial or loading dose and the one or more secondary or maintenance doses each contain the same amount of the IL-4R antagonist. In other embodiments, the initial dose comprises a first amount of the IL-4R antagonist, and the one or more secondary doses each comprise a second amount of the IL-4R antagonist. For example, the first amount of the IL-4R antagonist can be 1.5×, 2×, 2.5×, 3×, 3.5×, 4× or 5× or more than the second amount of the IL-4R antagonist. In some embodiments, one or more maintenance doses of the IL-4R antagonist are administered without a loading dose.

In some embodiments, a loading dose is a “split dose” that is administered as two or more doses (e.g., 2, 3, 4, or 5 doses) that are administered on separate days. In some embodiments, a loading dose is administered as a split dose wherein the two or more doses are administered at least about one week apart. In some embodiments, a loading dose is administered as a split dose wherein the two or more doses are administered about 1 week, 2 weeks, 3 weeks, or 4 weeks apart. In some embodiments, the loading dose is split evenly over the two or more doses (e.g., half of the loading dose is administered as the first portion and half of the loading dose is administered as the second portion). In some embodiments, the loading dose is split unevenly over the two or more doses (e.g., more than half of the loading dose is administered as the first portion and less than half of the loading dose is administered as the second portion).

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of IL-4R antagonist which is administered to a patient prior to the administration of the very next dose in the sequence with no intervening doses.

The methods of the disclosure may comprise administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose is administered at the same frequency as the other secondary doses. For example, each secondary dose may be administered to the patient 1 week, 2 weeks, 3 weeks, or 4 weeks after the immediately preceding dose. Similarly, in some embodiments involving multiple tertiary doses, each tertiary dose is administered at the same frequency as the other tertiary doses. For example, each tertiary dose may be administered to the patient 1 week, 2 weeks, 3 weeks, or 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, for a subject having eosinophilic esophagitis a therapeutically effective amount of an IL-4R antagonist (e.g., anti-IL-4R antibody) comprises 100 mg administered every two weeks (Q2W), if the subject is ≥5 kg to <15 kg in weight. In some embodiments, no loading dose is administered. In some embodiments, a loading dose is administered, e.g., a loading dose of 200 mg.

In some embodiments, for a subject having eosinophilic esophagitis a therapeutically effective amount of an IL-4R antagonist (e.g., anti-IL-4R antibody) comprises 200 mg administered every three weeks (Q3W), if the subject is ≥5 kg to <15 kg in weight. In some embodiments, no loading dose is administered. In some embodiments, a loading dose is administered, e.g., a loading dose of 400 mg.

In some embodiments, for a subject having eosinophilic esophagitis a therapeutically effective amount of an IL-4R antagonist (e.g., anti-IL-4R antibody) comprises 200 mg administered every four weeks (Q4W), if the subject is ≥5 kg to <15 kg in weight. In some embodiments, no loading dose is administered. In some embodiments, a loading dose is administered, e.g., a loading dose of 400 mg.

In some embodiments, for a subject having eosinophilic esophagitis a therapeutically effective amount of an IL-4R antagonist (e.g., anti-IL-4R antibody) comprises 200 mg administered every two weeks (Q2W), if the subject is ≥15 kg to <30 kg in weight. In some embodiments, no loading dose is administered. In some embodiments, a loading dose is administered, e.g., a loading dose of 400 mg.

In some embodiments, for a subject having eosinophilic esophagitis a therapeutically effective amount of an IL-4R antagonist (e.g., anti-IL-4R antibody) comprises 300 mg administered every four weeks (Q4W), if the subject is ≥15 kg to <30 kg in weight. In some embodiments, no loading dose is administered. In some embodiments, a loading dose is administered, e.g., a loading dose of 400 mg.

In some embodiments, for a subject having eosinophilic esophagitis a therapeutically effective amount of an IL-4R antagonist (e.g., anti-IL-4R antibody) comprises 300 mg administered every two weeks (Q2W), if the subject is ≥30 kg to <40 kg in weight. In some embodiments, no loading dose is administered. In some embodiments, a loading dose is administered, e.g., a loading dose of 600 mg.

In some embodiments, for a subject having eosinophilic esophagitis a therapeutically effective amount of an IL-4R antagonist (e.g., anti-IL-4R antibody) comprises 200 mg administered every two weeks (Q2W), if the subject is ≥30 kg to <40 kg in weight. In some embodiments, no loading dose is administered. In some embodiments, a loading dose is administered, e.g., a loading dose of 400 mg.

In some embodiments, for a subject having eosinophilic esophagitis a therapeutically effective amount of an IL-4R antagonist (e.g., anti-IL-4R antibody) comprises 300 mg administered every week (QW), if the subject is ≥40 kg in weight. In some embodiments, no loading dose is administered. In some embodiments, a loading dose is administered, e.g., a loading dose of 600 mg.

In some embodiments, for a subject having eosinophilic esophagitis a therapeutically effective amount of an IL-4R antagonist (e.g., anti-IL-4R antibody) comprises 300 mg administered every two weeks (Q2W), if the subject is ≥40 kg in weight. In some embodiments, no loading dose is administered. In some embodiments, a loading dose is administered, e.g., a loading dose of 600 mg.

In some embodiments, for a subject having eosinophilic esophagitis a therapeutically effective amount of an IL-4R antagonist (e.g., anti-IL-4R antibody) comprises 300 mg administered every two weeks (Q2W), if the subject is ≥30 kg to <60 kg in weight. In some embodiments, no loading dose is administered. In some embodiments, a loading dose is administered, e.g., a loading dose of 600 mg.

In some embodiments, for a subject having eosinophilic esophagitis a therapeutically effective amount of an IL-4R antagonist (e.g., anti-IL-4R antibody) comprises 200 mg administered every two weeks (Q2W), if the subject is ≥30 kg to <60 kg in weight. In some embodiments, no loading dose is administered. In some embodiments, a loading dose is administered, e.g., a loading dose of 400 mg.

In some embodiments, for a subject having eosinophilic esophagitis a therapeutically effective amount of an IL-4R antagonist (e.g., anti-IL-4R antibody) comprises 300 mg administered every week (QW), if the subject is ≥60 kg in weight. In some embodiments, no loading dose is administered. In some embodiments, a loading dose is administered, e.g., a loading dose of 600 mg.

In some embodiments, for a subject having eosinophilic esophagitis a therapeutically effective amount of an IL-4R antagonist (e.g., anti-IL-4R antibody) comprises 300 mg administered every two weeks (Q2W), if the subject is ≥60 kg in weight. In some embodiments, no loading dose is administered. In some embodiments, a loading dose is administered, e.g., a loading dose of 600 mg.

### Therapeutic Dosage Forms

In another aspect, the present disclosure provides therapeutic dosage forms of an IL-4R antagonist (e.g., an anti-IL-4R antibody or antigen-binding fragment thereof) for use in treating a subject having eosinophilic esophagitis as disclosed herein.

In some embodiments, the therapeutic dose is 100 mg of the IL-4R antagonist (e.g., anti-IL-4R antibody) and is for administration every two weeks (Q2W), if the subject is ≥5 kg to <15 kg in weight.

In some embodiments, the therapeutic dose is 200 mg of the IL-4R antagonist (e.g., anti-IL-4R antibody) and is for administration every three weeks (Q3W), if the subject is ≥5 kg to <15 kg in weight.

In some embodiments, the therapeutic dose is 200 mg of the IL-4R antagonist (e.g., anti-IL-4R antibody) and is for administration every four weeks (Q4W), if the subject is ≥5 kg to <15 kg in weight.

In some embodiments, the therapeutic dose is 200 mg of the IL-4R antagonist (e.g., anti-IL-4R antibody) and is for administration every two weeks (Q2W), if the subject is ≥15 kg to <30 kg in weight.

In some embodiments, the therapeutic dose is 300 mg of the IL-4R antagonist (e.g., anti-IL-4R antibody) and is for administration every four weeks (Q4W), if the subject is ≥15 kg to <30 kg in weight.

In some embodiments, the therapeutic dose is 300 mg of the IL-4R antagonist (e.g., anti-IL-4R antibody) and is for administration every two weeks (Q2W), if the subject is ≥30 kg to <40 kg in weight.

In some embodiments, the therapeutic dose is 200 mg of the IL-4R antagonist (e.g., anti-IL-4R antibody) and is for administration every two weeks (Q2W), if the subject is ≥30 kg to <40 kg in weight.

In some embodiments, the therapeutic dose is 300 mg of the IL-4R antagonist (e.g., anti-IL-4R antibody) and is for administration every week (QW), if the subject is ≥40 kg in weight.

In some embodiments, the therapeutic dose is 300 mg of the IL-4R antagonist (e.g., anti-IL-4R antibody) and is for administration every two weeks (Q2W), if the subject is ≥40 kg in weight.

In some embodiments, the therapeutic dose is 300 mg of the IL-4R antagonist (e.g., anti-IL-4R antibody) and is for administration every two weeks (Q2W), if the subject is ≥30 kg to <60 kg in weight.

In some embodiments, the therapeutic dose is 200 mg of the IL-4R antagonist (e.g., anti-IL-4R antibody) and is for administration every two weeks (Q2W), if the subject is ≥30 kg to <60 kg in weight.

In some embodiments, the therapeutic dose is 300 mg of the IL-4R antagonist (e.g., anti-IL-4R antibody) and is for administration every week (QW), if the subject is ≥60 kg in weight.

In some embodiments, the therapeutic dose is 300 mg of the IL-4R antagonist (e.g., anti-IL-4R antibody) and is for administration every two weeks (Q2W), if the subject is ≥60 kg in weight.

### EoE-Related Parameters

In some embodiments, the therapeutic methods disclosed herein result in an improvement in one or more endpoints or EoE-related parameters that are used to assess the presence or severity of EoE in a subject. Examples of EoE-related parameters include, but are not limited to: (a) change (e.g., reduction) in esophageal intraepithelial eosinophil count; (b) change in the severity and/or extent of histologic features in the esophagus, e.g., as measured using the Eosinophilic Esophagitis Histological Scoring System (EoE-HSS); (c) change in one or more esophageal characteristics, e.g., the absence, presence, or severity of edema, rings, exudates, furrows, and/or stricture, e.g., as measured using the EoE-EREFS; (d) change (e.g., normalization) in the levels of one or more EoE-associated biomarkers or in the EoE gene expression signature; (e) change (e.g., reduction) in frequency and/or intensity of dysphagia, e.g., as measured using the Dysphagia Symptom Questionnaire (DSQ), the Straumann Dysphagia Instrument (SDI), the Patient Global Impression of Change (PGIC) or Patient Global Impression of Severity (PGIS) of Dysphagia; or (f) change in the frequency and/or severity of other symptoms of EoE, e.g., as measured using the Pediatric EoE Sign/Symptom Questionnaire (PESQ) patient version (PESQ-P) or caregiver version (PESQ-C), the Pediatric EoE Impact Scale (PEIS) patient version (PEIS-P) or caregiver version (PEIS-C), the Global Impression of Change (GIC) patient version (GIC-P), caregiver version (GIC-C), or clinician version (GIC-Clin), the Global Impression of Severity (GIS) patient version (GIS-P), caregiver version (GIS-C), or clinician version (GIS-Clin), or the Pediatric Esophagitis Symptom Score (PEESS). Methods for assessing these and other EoE-related parameters are described in the Examples section below and are also disclosed in WO 2019/028367 and WO 2021/237110, incorporated by reference herein.

To determine whether an EoE-related parameter has “improved,” the parameter is quantified at baseline (e.g., prior to the start of treatment with the IL-4R antagonist) and at one or more timepoints after administration of the IL-4R antagonist. For example, an EoE-related parameter may be measured at day 1, day 2, day 3, day 4, day 5, day 6, day 7, day 8, day 9, day 10, day 11, day 12, day 14, day 15, day 22, day 25, day 29, day 36, day 43, day 50, day 57, day 64, day 71, day 85; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with a pharmaceutical composition of the present disclosure. The difference between the value of the parameter at a particular timepoint following initiation of treatment and the value of the parameter at baseline is used to establish whether there has been an improvement in the EoE-related parameter.

In some embodiments, treatment of a subject with an IL-4R antagonist (e.g., an anti-IL-4R antibody) results in an improvement (e.g., reduction) in peak esophageal intraepithelial eosinophil count. “Peak esophageal intraepithelial eosinophil count” refers to the number of eosinophils contained within one high power field (hpf). In some embodiments, treatment with an IL-4R antagonist results in a decrease in peak esophageal intraepithelial eosinophil count relative to baseline (e.g., a subject's peak count prior to the onset of treatment). In some embodiments, treatment with an IL-4R antagonist results in peak esophageal intraepithelial eosinophil count of at least 10%, 15%, 20%, 25%, 30%, 35%, 40%, 45%, 50% or more relative to baseline. In some embodiments, treatment with an IL-4R antagonist results in a decrease in peak esophageal intraepithelial eosinophil count to less than 10 eos/hpf or less than 6 eos/hpf. In some embodiments, treatment with an IL-4R antagonist results in a decrease in peak esophageal intraepithelial eosinophil count to ≥6 eos/hpf, ≥5 eos/hpf, ≥4 eos/hpf, ≥3 eos/hpf, ≥2 eos/hpf, or ≤1 eos/hpf. In some embodiments, treatment with an IL-4R antagonist results in histological disease remission, as measured by peak esophageal intraepithelial eosinophil count of 56 eos/hpf. In some embodiments, the change in peak esophageal intraepithelial eosinophil count measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment of a subject with an IL-4R antagonist (e.g., an anti-IL-4R antibody) results in an improvement in one or more histological features of EoE. In some embodiments, treatment of a subject with an IL-4R antagonist (e.g., an anti-IL-4R antibody) results in an improvement in EoE-HSS score. The EoE-HSS is a validated instrument that generates separate severity (grade) and extent (stage) disease scores. The score is used to measure 8 histologic features (parameters) of EoE from 3 different regions (proximal, mid, and distal) of the esophagus (Collins, et al., Dis Esophagus, 2017, 30:1-8). The 8 parameters include: eosinophil density, basal zone hyperplasia, eosinophil abscesses, eosinophil surface layering, dilated intercellular spaces, surface epithelial alteration, dyskeratotic cells, and lamina propria fibrosis. A scale of 0-3 is used for each parameter, both grade and stage (with 0 being least inflamed, normal). In some embodiments, treatment with an IL-4R antagonist results in a decrease in EoE-HSS score of at least 10%, 15%, 20%, 25%, 30%, 35%, 40%, 45%, 50% or more relative to baseline (e.g., a subject's EoE-HSS score prior to the onset of treatment). In some embodiments, treatment with an IL-4R antagonist results in a decrease in EoE-HSS composite score, grade score, and/or stage score. In some embodiments, the change in EoE-HSS score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment of a subject with an IL-4R antagonist (e.g., an anti-IL-4R antibody) results in an improvement in one or more endoscopic features of EoE. In some embodiments, treatment of a subject with an IL-4R antagonist (e.g., an anti-IL-4R antibody) results in an improvement in EoE-EREFS score. The EoE-EREFS (edema, rings, exudates, furrows, strictures) is validated scoring system for inflammatory and remodeling features of disease that is used to measure the endoscopically identified EoE esophageal mucosal inflammatory and remodeling features (Hirano, et al., Gut, 2013, 62:489-495). This instrument includes a total of 17 items related to the presence and severity of esophageal features. The specific esophageal features include: rings (concentric rings around esophagus—absent, mild, moderate, severe, not applicable); strictures (narrowing of the esophagus—yes, no, not applicable); diameter of the stricture (if applicable); exudates (refer to white plaques—absent, mild, severe), furrows (vertical lines down the esophagus—absent, present); edema (loss of vascular markings of the mucosa—absent, present); crepe paper esophagus (absent, present); overall general appearance incorporating all endoscopically identified EoE findings (i.e., fixed rings, strictures, whitish exudates, furrowing, edema, and crepe paper mucosa). In addition, mucosal changes associated with gastroesophageal reflux disease are recorded using the Los Angeles classification system for erosions (No Erosions or LA Classification A, B, C, D). In some embodiments, treatment with an IL-4R antagonist results in a decrease in EoE-EREFS score of at least 10%, 15%, 20%, 25%, 30%, 35%, 40%, 45%, 50% or more relative to baseline (e.g., a subject's EoE-EREFS score prior to the onset of treatment). In some embodiments, the change in EoE-EREFS score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist. In some embodiments, treatment with an IL-4R antagonist results in an improvement in one or more EoE-EREFS subscores.

In some embodiments, treatment of a subject with an IL-4R antagonist (e.g., an anti-IL-4R antibody) results in an improvement in symptoms of dysphagia. In some embodiments, treatment results in a change (e.g., reduction) in the frequency and/or intensity of dysphagia in a subject, e.g., as measured using the Dysphagia Symptom Questionnaire (DSQ), the Straumann Dysphagia Instrument (SDI), the Patient Global Impression of Change (PGIC) or Patient Global Impression of Severity (PGIS) of Dysphagia. In some embodiments, treatment results in a reduction in the frequency of episodes of dysphagia per week, e.g., a decrease of at least about 10%, 15%, 20%, 25%, 30%, 35%, 40%, 45%, 50% or more relative to baseline (e.g., the subject's average frequency of episodes of dysphagia per week prior to the onset of treatment). In some embodiments, treatment results in an improvement in DSQ score or SDI. The DSQ is a validated PRO that has been used in clinical studies to measure the frequency and intensity of dysphagia. See, Hudgens, et al., J Patient Rep Outcomes, 2017, 1(1):3, doi:10.1186/s41687-017-0006-5. The SDI is a non-validated patient reported outcome (PRO) that has been used in clinical studies to determine the frequency and intensity of dysphagia (Straumann, Gastroenterology, 2010, 139:1526-1537). In some embodiments, treatment with an IL-4R antagonist results in a decrease in DSQ score of at least 3, 4, 5, 6, 7, 8, 9, 10, 11, 12, 13, 14, 15, 16, 17, 18, 19, 20 or more points relative to baseline. In some embodiments, treatment with an IL-4R antagonist results in a decrease in DSQ score or SDI score of at least 10%, 15%, 20%, 25%, 30%, 35%, 40%, 45%, 50% or more relative to baseline. In some embodiments, the change in score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist. In some embodiments, treatment with an IL-4R antagonist reduces symptoms of dysphagia in a subject (e.g., as measured by change in absolute DSQ score or percentage decrease in DSQ or SDI score relative to a baseline value for the subject) within about 4 weeks, 5 weeks, 6 weeks, 7 weeks, 8 weeks, 9 weeks, or 10 weeks of starting treatment with the IL-4R antagonist.

In some embodiments, treatment of a subject with an IL-4R antagonist (e.g., an anti-IL-4R antibody) results in a normalization of one or more EoE-associated biomarkers, an EoE gene signature, a Type 2 inflammation gene signature, and/or a Normalized Enrichment Score (NES) calculated for a set of EoE-associated genes. In some embodiments, treatment of a subject with an IL-4R antagonist suppresses an EoE gene signature, a Type 2 inflammation gene signature, and/or an NES calculated for a set of EoE-associated or Type 2 inflammation genes. As used herein, the term “EoE-associated biomarker” refers to a biological response, cell type, parameter, protein, polypeptide, enzyme, enzyme activity, metabolite, nucleic acid, carbohydrate, or other biomolecule which is present or detectable in an EoE patient at a level or amount that is different from (e.g., greater than or less than) the level or amount of the marker present or detectable in a non-EoE patient. In some embodiments, the EoE-associated biomarker is a gene associated with fibrosis, tissue remodeling, or epithelial barrier function. Exemplary EoE-associated biomarkers include, but are not limited to, e.g., esophagus eosinophils, eotaxin-3 (CCL26), periostin (POSTN), serum IgE (total and allergen-specific), serum IgG (total and allergen-specific), arachidonate 15-lipoxygenase (ALOX15), IL-13, IL-5, serum thymus and activation regulated chemokine (TARC; CCL17), thymic stromal lymphopoietin (TSLP), serum eosinophilic cationic protein (ECP), collagen genes (e.g., COL4A3, COL4A4, COL4A6, COL8A2, COL14A1, and COL21A1), calpain 14, desmoglein-1 (DSG1), filaggrin (FLG), signal transducer and activator of transcription 6 (STAT6), serine peptidase inhibitor Kazal-type 5 (SPINK5), SPINK7, SPINK8, interleukin 4 receptor (IL-4R), eosinophil-associated genes (e.g., CLC and SIGLEC8), anoctamin-1 (ANO1), cathepsin C (CTSC), C-C chemokine receptor type 3 (CCR3), and eosinophil-derived neurotoxin (EDN). The term “EoE gene signature” refers to a differential gene expression profile of esophageal biopsies of EoE patients compared to healthy controls, and is also referred to as an “EoE disease transcriptome” (Sherrill, et al., Genes Immun, 2014, 15:361-369). In some embodiments, an EoE gene signature is a smaller gene set of the published EoE disease transcriptome, such as the EoE diagnostic panel (EDP, clinically available as EoGenius™, Inform Diagnostics, USA). A “Type 2 inflammation gene signature” refers to a transcriptome for a set of genes associated with type 2 inflammation. Exemplary Type 2 inflammation-associated genes include, but are not limited to, CCL26, ALOX15, CCR3, and IL1RL1. An exemplary gene list for a Type 2 inflammation gene signature is shown in WO 2021/237110. A Normalized Enrichment Score (NES) reflects the degree to which the activity level of a set of transcripts is overrepresented at the extremes (top or bottom) of the entire ranked list of transcripts within a sample and is normalized by accounting for the number of transcripts in the set (Subramanian, et al., Proc Natl Acad Sci USA, 2005, 102:15545-50) (Barbie, et al., Nature, 2009, 462:108-112).

In some embodiments, the EoE-associated biomarkers, EoE gene signature, Type 2 inflammation gene signature, and/or NES are determined using a tissue sample from the subject (e.g., esophageal pinch biopsy samples from the proximal, mid, and/or distal regions). In some embodiments, treatment of a subject with an IL-4R antagonist results in a normalization of one or more EoE-associated biomarkers, an EoE gene signature, a Type 2 inflammation gene signature, and/or an NES, relative to baseline (e.g., a subject's level of expression of the EoE-associated biomarker(s), EoE gene signature, or NES prior to the onset of treatment), e.g., as measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist. In some embodiments, treatment of a subject with an IL-4R antagonist suppresses a NES for one or more EoE-associated biomarkers, an EoE gene signature, or a Type 2 inflammation gene signature, relative to baseline (e.g., a subject's NES prior to the onset of treatment), e.g., as measured at day 8, 15, 22, 25, 29, 36, 43, 50, 57, 64, 71, 85, 113 or later following administration of the IL-4R inhibitor, or after 2, 4, 6, 8, 10, 12, 14, 16, 18, 20, or 24 weeks of treatment with the IL-4R antagonist.

In some embodiments, treatment of a subject with an IL-4R antagonist (e.g., an anti-IL-4R antibody) results in an improvement in one or more other signs or symptoms of EoE or in health-related quality of life.

In some embodiments, treatment results in an improvement in PESQ score. The PESQ has a patient version (PESQ-P) that is intended for use with EoE patients aged 8-12 years, and a caregiver version (PESQ-C) that is intended for use by caregivers of patients with EoE from 1-11 years old. Both questionnaires measure the same concepts; the PESQ-P assesses the frequency, duration, and/or severity of symptoms and the PESQ-C assesses the presence/absence of the signs/symptoms. These symptoms include stomach pain, heartburn, acid reflux, regurgitation, vomiting, refusal to eat, trouble swallowing, and food getting stuck in the throat. See, Kamat, et al., J Allergy Clin Immunol: In Practice, 2022, ISSN: 2213-2198. In some embodiments, treatment with an IL-4R antagonist results in a decrease in PESQ score of at least 10%, 15%, 20%, 25%, 30%, 35%, 40%, 45%, 50% or more relative to baseline. In some embodiments, the change in PESQ score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist. In some embodiments, treatment with an IL-4R antagonist results in a decrease in the number of days, or the number of total segments within a day (e.g., night, morning, afternoon, evening) with 1 or more EoE signs as measured by PESQ-P or PESQ-C. In some embodiments, treatment with an IL-4R antagonist results in an improvement in one or more signs/symptoms measured by PESQ.

In some embodiments, treatment results in an improvement in PEIS score. The PEIS has a patient version (PEIS-P) that is intended to be completed independently by pediatric EoE patients aged 8-12 years, and a caregiver version (PEIS-C) that is intended for use by caregivers of patients with EoE from 1-11 years old. The PEIS-P assesses the impact of EoE on patients' health-related during the past 1 week, while the PEIS-C assesses the impact of the pediatric patient's EoE on caregiver anxiety, social and professional activities, activities of daily living, and relationships during the past 1 week. In some embodiments, treatment with an IL-4R antagonist results in a decrease in PEIS score of at least 10%, 15%, 20%, 25%, 30%, 35%, 40%, 45%, 50% or more relative to baseline. In some embodiments, the change in PEIS score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment results in an improvement in GIC score. The GIC is a single-item outcome measure that has a patient version (GIC-P) intended to be completed independently by pediatric EoE patients aged 8-12 years, a caregiver version (GIC-C) intended to be completed independently by caregivers of patients with EoE from 1-11 years old, and a clinician version (GIC-Clin) intended to be completed independently by a physician for patients with EoE from 1-11 years old. All measure the same concept: the GIC-P assesses the patient's impression about the overall change (improvement or worsening) in his/her EoE condition since study treatment initiation, while the GIC-C and GIC-Clin assess the caregiver or physician's impression, respectively, about the overall change (improvement or worsening) in the pediatric patient's EoE condition since study treatment initiation. In some embodiments, treatment with an IL-4R antagonist results in an improvement (e.g., decrease) in GIC score of at least 10%, 15%, 20%, 25%, 30%, 35%, 40%, 45%, 50% or more relative to baseline. In some embodiments, the change in GIC score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment results in an improvement in GIS score. The GIS is a single-item outcome measure that has a patient version (GIS-P) intended to be completed independently by pediatric EoE patients aged 8-12 years, a caregiver version (GIS-C) intended to be completed independently by caregivers of patients with EoE from 1-11 years old, and a clinician version (GIS-Clin) intended to be completed independently by a physician for patients with EoE from 1-11 years old. All measure the same concept: the GIS-P assesses the patient's impression about the overall severity of his/her EoE condition during the past 1 week, while the GIS-C and GIS-Clin assess the caregiver or physician's impression, respectively, about the overall severity of the pediatric patient's EoE condition during the past 1 week. In some embodiments, treatment with an IL-4R antagonist results in an improvement (e.g., decrease) in GIS score of at least 10%, 15%, 20%, 25%, 30%, 35%, 40%, 45%, 50% or more relative to baseline. In some embodiments, the change in GIS score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment results in an improvement in PEESS score. The PEESSv2.0 is a caregiver reported outcomes measure which assesses the frequency and severity of EoE symptoms among pediatric patients (Franciosi, 2011). The PEESSv2.0 caregiver version consists of 20 items and has a one-month recall period. The total PEESSv2.0 score ranges from 0 to 100; higher scores indicate greater symptom burden of among pediatric EoE patients. In some embodiments, treatment with an IL-4R antagonist results in a decrease in PEESS score of at least 10%, 15%, 20%, 25%, 30%, 35%, 40%, 45%, 50% or more relative to baseline. In some embodiments, treatment with an IL-4R antagonist results in a decrease in PEESS score of at least 3, 4, 5, 6, 7, 8, 9, 10, 11, 12, 13, 14, 15, 16, 17, 18, 19, 20 or more points relative to baseline. In some embodiments, the change in PEESS score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment with an IL-4R antagonist results in improved weight gain, relative to the patient's baseline weight, as compared to a control (e.g., a placebo treated patient). In some embodiments, treatment with an IL-4R antagonist results in an increase in body weight of at least 0.5%, 1%, 1.5%, 2%, 2.5%, 3% or more relative to baseline. In some embodiments, the change in body weight is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

### Combination Therapies

In some embodiments, the methods of the present disclosure comprise administering to the subject (e.g., a subject≥1 year and <12 years of age) an IL-4R antagonist according to the disclosure (e.g., an anti-IL-4R antibody) in combination with one or more additional therapeutic agents. As used herein, the expression “in combination with” means that the additional therapeutic agents are administered before, after, or concurrent with the pharmaceutical composition comprising the IL-4R antagonist. The term “in combination with” also includes sequential or concomitant administration of IL-4R antagonist and a second therapeutic agent or therapy.

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the IL-4R antagonist. Administration “concurrent” or with the pharmaceutical composition comprising the IL-4R antagonist means that the additional therapeutic agent is administered to the subject in a separate dosage form within less than about 10 minutes (before, after, or at the same time) of administration of the pharmaceutical composition comprising the IL-4R antagonist, or administered to the subject as a single combined dosage formulation comprising both the additional therapeutic agent and the IL-4R antagonist.

In some embodiments, the second therapeutic agent or therapy is an IL-1p inhibitor, an IL-5 or IL-5R inhibitor (e.g., an anti-IL-5 or anti-IL-5R antibody such as benralizumab, mepolizumab, or reslizumab), an IL-9 inhibitor, an IL-13 inhibitor (e.g., an anti-IL-13 antibody such as tralokinumab, RPC4046, or QAX576), an IL-17 inhibitor, an IL-25 inhibitor, a TNFα inhibitor (e.g., an anti-TNFα antibody such as infliximab or adalimumab), an eotaxin-3 inhibitor, an IgE inhibitor (e.g., an anti-IgE antibody such as omalizumab), a TSLP inhibitor (e.g., an anti-TSLP antibody such as tezepelumab), a CRTH2 inhibitor, a Siglec-8 inhibitor, a prostaglandin D2 inhibitor, an integrin inhibitor (e.g., an integrin α4β7 inhibitor such as vedolizumab), an eotaxin inhibitor, an immunosuppressant, a topical corticosteroid, an oral corticosteroid, a systemic corticosteroid, an inhaled corticosteroid, a glucocorticoid, a PPI, a decongestant, an antihistamine, a non-steroidal anti-inflammatory drug (NSAID), esophagus dilation, a feeding tube, allergen removal, or diet management. In some embodiments, the IL-4R antagonist is used in combination with diet management, allergen removal, or a feeding tube. In some embodiments, the IL-4R antagonist is used in combination with a corticosteroid (e.g., a swallowed topical corticosteroid). In some embodiments, the IL-4R antagonist is used in combination with a PPI, e.g., omeprazole, esomeprazole, lansoprazole, dexlansoprazole, rabeprazole, or pantoprazole.

In some embodiments, administration of the IL-4R antagonist reduces dependence on or the need for using a concurrent therapy (e.g., a PPI, corticosteroid, glucocorticoid, or a feeding tube). In some embodiments, administration of the IL-4R antagonist in combination with the second therapy (e.g., PPI, corticosteroid, glucocorticoid, or feeding tube) reduces the amount of the second therapy used by the patient by at least 20%, at least 30%, at least 40% or at least 50% as compared to the amount used by the subject before treatment with the IL-4R antagonist. In some embodiments, administration of the IL-4R antagonist eliminates the need for the second therapy.

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Clinical Trial Investigating the Efficacy and Safety of Dupilumab in Pediatric Patients with Eosinophilic Esophagitis

### Study Design and Objectives

This study is a Phase 3, multicenter, randomized, three-part, double-blind, placebo-controlled study to evaluate the efficacy, safety, tolerability, pharmacokinetics, and immunogenicity of dupilumab in pediatric patients (ages≥1 year to <12 years) with active EoE. The study consists of a screening period of up to 85 days, a double-blind 16-week treatment period (Part A), an open-label 36-week extended treatment period (Part B), a 108-week open-label extension period (Part C), and a 12-week follow-up period. Dupilumab is a fully human anti-IL-4R antibody comprising a heavy chain comprising the amino acid sequence of SEQ ID NO:9 and a light chain comprising the amino acid sequence of SEQ ID NO:10; an HCVR/LCVR amino acid sequence pair comprising SEQ ID NOs:1/2; and heavy and light chain CDR sequences comprising SEQ ID NOs:3-8.

The primary objective of the study is to demonstrate the efficacy of dupilumab treatment compared with placebo in pediatric patients with active EoE based on histologic improvement meeting validated histologic criteria. The secondary objectives include: to demonstrate the efficacy of dupilumab compared to placebo in pediatric patients with active EoE after 16 weeks of treatment as assessed by endoscopic visual measurements of disease activity using the Eosinophilic Esophagitis-Endoscopic Reference Score (EoE-EREFS) and histologic abnormalities as measured by the EoE Histology Scoring System (EoE-HSS); to evaluate the safety, tolerability, and immunogenicity of dupilumab treatment for up to 16 weeks in pediatric patients with active EoE; to evaluate the effects of dupilumab on transcriptomic signatures associated with EoE and type 2 inflammation; to study the effects of dupilumab on the type 2 inflammation gene expression signature; to evaluate the concentration-time profile of functional dupilumab in serum in this population; to assess efficacy of long-term (up to 160 weeks) dupilumab treatment; to assess the impact of dupilumab treatment on changes in weight and growth during the extended active period and open-label extension period of the study; to assess safety, tolerability, and immunogenicity of long-term (up to 160 weeks) dupilumab treatment; and to evaluate the impact of dupilumab treatment on EoE signs and symptoms.

This study is being conducted in accordance with the provisions of the Declaration of Helsinki, the International Conference on Harmonization Good Clinical Practices guideline, and applicable regulatory requirements. The protocol was reviewed and approved by institutional review boards/ethics committees at all sites. For all patients, written informed consent is obtained from a parent or legal guardian.

### Patient Population

This study includes pediatric patients (aged≥1 year to <12 years at the time of screening visit) who have active EoE.

Inclusion Criteria: A patient needed to meet the following criteria to be eligible for inclusion in the study: (1) male or female, ≥1 year to <12 years of age; (2) a documented diagnosis of EoE by endoscopic biopsy prior to screening, as demonstrated by intraepithelial eosinophilic infiltration (peak eosinophil count 15 eos/hpf) (400×) from at least 1 esophageal region and performed after at least 8 weeks of treatment with a PPI regimen. If the patient discontinued PPI therapy, the biopsy must have been performed within 2 weeks of the date of discontinuation. If a prior (documented) endoscopic biopsy meeting these criteria is not available (or no prior biopsy is available), patients who meet other clinical and laboratory eligibility criteria will undergo treatment with a PPI regimen for at least 8 weeks during the screening period before their baseline endoscopy/biopsies. (3) baseline endoscopic biopsies with a demonstration on central reading of intraepithelial eosinophilic infiltration (peak eosinophil count≥15 eos/hpf) in at least 2 of the 3 biopsied esophageal regions (proximal, mid, or distal); (4) history (by patient or caregiver report) of symptom(s) determined by the investigator to be the result of EoE (e.g., abdominal pain, chest pain, acid reflux, food regurgitation, dysphagia, vomiting, or refusal to eat) in the month prior to screening; (5) patients≥8 to <12 years of age and caregiver or legal guardians of all patients must be able to understand and complete the study requirements and study-related questionnaires. At least 8 out of 14 days of eDiary for the PESQ-C should be completed prior to baseline/visit 3. (6) parents or legal guardians must provide signed informed consent. Assent should be collected from patient, if applicable, as per local regulatory (competent authority/ethics) guidelines, based upon the age and level of maturity of the patient.

Exclusion Criteria: The following were exclusion criteria for Part A of the study: (1) body weight<5 kg or ≥60 kg at screening; (2) prior participation in a dupilumab clinical trial, or past or current treatment with dupilumab; (3) initiation or change of a food-elimination diet regimen or re-introduction of a previously eliminated food group in the 6 weeks prior to screening. Patients on a food-elimination diet must remain on the same diet throughout the study. (4) other causes of esophageal eosinophilia or the following conditions: eosinophilic gastroenteritis, hypereosinophilic syndrome, and eosinophilic granulomatosis with polyangiitis (Churg-Strauss syndrome); (5) active Helicobacter pylori infection; (6) helminthic infection; (7) history of Crohn's disease, ulcerative colitis, celiac disease, or prior esophageal surgery; (8) any esophageal stricture unable to be passed with a standard, diagnostic, upper endoscope or any critical esophageal stricture that requires dilation at screening; (9) treatment with swallowed topical corticosteroids within 8 weeks prior to baseline standard of care endoscopy; (10) treatment with subcutaneous immunotherapy (SCIT) unless on a stable maintenance dose for at least 1 year; (11) prior treatment with sublingual immunotherapy (SLIT), epicutaneous immunotherapy (EPIT), or oral immunotherapy (OIT); (12) initiation, discontinuation, or change in the dosage regimen of the following medications within 8 weeks prior to the baseline endoscopy: PPIs, leukotriene inhibitors, and nasal and/or inhaled corticosteroids; (13) treatment with systemic immunosuppressant/immunomodulating drugs, including but not limited to systemic corticosteroids, mepolizumab, omalizumab, cyclosporine, mycophenolate-mofetil, interferon-gamma [IFN-γ], Janus kinase inhibitors, azathioprine, and methotrexate within 3 months prior to screening (Note: one-time use of a corticosteroid as a part of the anesthetic preparation used during each endoscopy procedure is allowed); (14) treatment with an investigational drug within 2 months or within 5 half-lives (if known), whichever is longer, prior to visit 1; (15) history of bleeding disorders or esophageal varices that, in the opinion of the investigator, would put the patient at undue risk for significant complications from an endoscopy procedure; (16) planned or anticipated use of any prohibited medications and procedures during the study; (17) planned or anticipated major surgical procedure during the study; (18) treatment with a live (attenuated) vaccine within 4 weeks prior to the baseline visit; (19) active parasitic infection or suspected parasitic infection, unless clinical and (if necessary) laboratory assessments have ruled out active infection before randomization; (20) chronic or acute infection requiring treatment with systemic antibiotics, antivirals, or antifungals within 2 weeks before baseline visit (Note: patients may be re-screened after the infection resolves); (21) known or suspected immunodeficiency disorder, including history of invasive opportunistic infections (e.g., tuberculosis [TB], non-tuberculous mycobacterial infections, histoplasmosis, listeriosis, coccidioidomycosis, pneumocystosis, aspergillosis) despite infection resolution, or otherwise recurrent infections of abnormal frequency, or prolonged infections suggesting an immune-compromised status, as judged by the investigator; (22) known history of human immunodeficiency virus (HIV) infection or positivity for hepatitis B or hepatitis C antigens; (23) on current treatment for hepatic disease including but not limited to acute or chronic hepatitis, cirrhosis, or hepatic failure, or has evidence of liver disease as indicated by persistent (confirmed by repeated tests≥2 weeks apart) elevated transaminases (alanine aminotransferase [ALT] and/or aspartate aminotransferase [AST]) more than 3 times the upper limit of normal [ULN] during the screening period); (24) any of the following abnormal lab values at screening: platelets<100×10³/μL, neutrophils<1.5×10³/μL, or estimated glomerular filtration rate (eGFR)<30 mL/min/1.73 m2; (25) severe concomitant illness(es) that, in the investigator's judgment, would adversely affect the patient's participation in the study. Examples include but are not limited to short life expectancy, uncontrolled diabetes, cardiovascular conditions (e.g., NYHA Class Ill or IV cardiac failure), severe renal conditions (e.g., patients on dialysis), hepatobiliary conditions (e.g., Child-Pugh class B or C), neurological conditions (e.g., demyelinating diseases), active major autoimmune diseases (e.g., lupus, inflammatory bowel disease, rheumatoid arthritis, etc.), and other severe endocrinologic, gastrointestinal, metabolic, pulmonary, or lymphatic diseases. (26) history of malignancy within 5 years prior to screening; (27) history of alcohol or drug abuse within 6 months prior to screening, in the opinion of the investigator; (28) any other medical or psychological condition including relevant laboratory abnormalities at screening that, in the opinion of the investigator, suggest a new and/or insufficiently understood disease, may present an unreasonable risk to the study patient as a result of his/her participation in this clinical trial, may make patient's participation unreliable, or may interfere with study assessments; (29) patient's immediate family is a member of the investigational team; (30) patient is female who is pregnant, breastfeeding, or planning to become pregnant or breastfeed during the study; (31) patient is of childbearing potential who is unwilling to practice highly effective contraception prior to the initial dose/start of the first treatment, during the study, and for at least 12 weeks after the last dose; (32) female patients who experience menarche and who are unwilling to follow the precautions for women of childbearing potential (WOCBP); (33) known systemic hypersensitivity to dupilumab or the excipients of the drug product.

The following are exclusion criteria for Part B of the study (extended active treatment): (1) patients who, during the double-blind treatment period, developed an SAE and/or AE deemed related to study drug and which led to discontinuation of investigational product (patients who are prematurely discontinued from study drug due to lack of efficacy are eligible to enter Part B); (2) patients who, during the double-blind treatment period, were prematurely withdrawn because of a protocol violation, poor compliance, or inability to complete required study assessments; (3) patients who did not undergo endoscopy with biopsies at week 16 or prior to receiving rescue treatment; (4) female patients who experience menarche and who are unwilling to follow the precautions for WOCBP.

The following are exclusion criteria for Part C of the study (OLE) for patients with uninterrupted participation in the study: (1) patients who, during the extended active treatment period, developed an SAE and/or AE deemed related to study drug and which led to discontinuation of investigational product (patients who are prematurely discontinued from study drug due to lack of efficacy are eligible to enter Part C); (2) patients who, during the extended active treatment period, were prematurely withdrawn because of a protocol violation, poor compliance, or inability to complete required study assessments; (3) patients who did not undergo endoscopy with biopsies at week 52 or prior to receiving rescue treatment; (4) female patients who experience menarche and who are unwilling to follow the precautions for WOCBP.

### Study Treatments

The study drug treatments are as follows:
Dupilumab 150 mg/mL: Each 2.25 mL single-use, prefilled glass syringe with snap-off cap delivers 300 mg of study drug (2.0 mL of a 150 mg/mL solution).
Dupilumab 175 mg/mL: Each 1.14 mL single-use, prefilled glass syringe with snap-off cap delivers 200 mg of study drug (1.14 mL of a 175 mg/mL solution).
Dupilumab 150 mg/mL: Each 0.67 mL single-use, prefilled glass syringe with snap-off cap delivers 100 mg of study drug (0.67 mL of a 150 mg/mL solution).
Placebo-matching dupilumab is prepared in the same formulation without the addition of protein (i.e., active substance, anti-IL-4Rα monoclonal antibody). Three matching placebo formulations will be used:
   2 mL placebo matching 300 mg dupilumab formulation
   1.14 mL placebo matching 200 mg dupilumab formulation
   0.67 mL placebo matching 100 mg dupilumab formulation

Part A is the double-blind, placebo-controlled portion of the study. In Part A, patients are randomized 1:1:1: to receive either higher exposure dupilumab or lower exposure dupilumab (in a tiered, weight-based dosing schema based on body weight at baseline) or placebo over 16 weeks by subcutaneous (SC) administration.

The Part A dosing regimens are as follows:

### Higher Exposure Dupilumab Regimens (Part A):

100 mg Q2W in patients who weigh≥5 kg to <15 kg
200 mg Q2W in patients who weigh 15 kg to <30 kg
300 mg Q2W in patients who weigh≥30 kg to <60 kg

### Lower Exposure Dupilumab Regimens (Part A):

200 mg Q4W in patients who weigh≥5 kg to <15 kg
300 mg Q4W in patients who weigh 15 kg to <30 kg
200 mg Q2W in patients who weigh≥30 kg to <60 kg
   NOTE: In Part A, patients will receive dupilumab injections at the frequency of Q2W or Q4W with matching placebo alternating with dupilumab doses so the injection frequency will be Q2W for both groups for regimen-blinding purposes per treatment assignment in Part A. Placebo for Part A: Dose matching placebo Q2W or alternating with dupilumab Q4W so the frequency will be Q2W for both groups for regimen-blinding purposes in Part A.

Part B is the extended active treatment portion of the study. All patients (Part A active and placebo) will receive dupilumab based on body weight at visit 8 (week 16, end of double-blinded treatment period or start of extended treatment period) per the higher and lower exposure group to which they were assigned at randomization. If weight tier has increased at visit 12/week 32, patients will be re-assigned to an extended active treatment regimen based on weight tiered dosing regimens below:

### Higher Exposure Dupilumab Regimens (Part B):

100 mg Q2W in patients who weigh≥5 kg to <15 kg
200 mg Q2W in patients who weigh≥15 kg to <30 kg
300 mg Q2W in patients who weigh≥30 kg to <60 kg
300 mg QW in patients who weigh≥60 kg

### Lower Exposure Dupilumab Regimens (Part B):

200 mg Q4W in patients who weigh≥5 kg to <15 kg
300 mg Q4W in patients who weigh≥15 kg to <30 kg
200 mg Q2W in patients who weigh≥30 kg to <60 kg
300 mg Q2W in patients who weigh≥60 kg
   NOTE: In Part B, study drug or matching placebo will be administered Q2W for all patients in <60 kg weight-groups and QW for all patients in ≥60 kg weight-groups. Patients will receive alternating doses of dupilumab or placebo Q2W for patients<60 kg assigned to Q4W regimens or QW for patients 60 kg assigned to Q2W regimens for regimen-blinding purposes. Placebo for Part B: Dose matching placebo alternating with dupilumab Q2W or Q4W so the frequency will be Q2W for the weight-based groups<60 kg and QW for the weight-based group of ≥60 kg for regimen-blinding purposes in Part B.

Part C is the open-label extension period of the study. All patients (Part C) will receive higher exposure dupilumab regimens based on body weight at visit 17 (week 52, end of extended active treatment period) or start of open-label period (for re-entry patients). All patients who received lower exposure dupilumab during Part B will be re-assigned to higher exposure dupilumab regimens at the start of the open-label (Part C) period. If weight increases place a patient into a higher weight tier at visit 17/week 52 (or start of open-label period for re-entry patients) or at specified in-clinic visits in Part C, the patient will be re-assigned to an open-label treatment regimen based on the weight tiered dosing regimen specified below.

### Higher Exposure Dupilumab Regimens (Part C):

200 mg Q3W in patients who weigh≥5 kg to <15 kg
200 mg Q2W in patients who weigh 15 kg to <30 kg
300 mg Q2W in patients who weigh 30 kg to <60 kg
300 mg QW in patients who weigh 60 kg
   NOTE: In Part C, only active study drug (dupilumab) will be administered. No matching placebo will be administered in Part C. For Part C, patients located in a country where dupilumab is commercially available for treatment of EoE in patients 12 years old will be provided treatment with study drug until they are both (1) at least 12 years of age and (2) weigh at least 40 kg or the lowest weight for which the indication is approved for EoE. These patients will have an end of treatment visit at the next scheduled study visit upon meeting both criteria, followed by an end of study follow-up visit after 12 weeks.

Background treatments: Patients who undergo a trial of PPI therapy prior to screening or during the screening period of Part A have the choice either to remain on the PPI regimen during the entire study or stop the PPI regimen prior to baseline; they then must remain off of PPIs during the entire study.

Rescue treatments: If medically necessary (e.g., for treatment of intolerable EoE symptoms), rescue medications (systemic and/or swallowed topical corticosteroids) or emergency esophageal dilation are allowed for study patients. An endoscopy with biopsy will be performed prior to the initiation of rescue therapy. Patients who undergo an endoscopy with biopsy due to the initiation of rescue therapy will not undergo the subsequent scheduled endoscopy/biopsy at week 16 and/or week 52. Patients who receive rescue treatment during the double-blind period of the study will not be eligible for the extended active treatment period unless an endoscopy with biopsy is performed prior to the initiation of rescue treatment. Patients receiving rescue therapy may continue to receive study drug. They will remain blinded and will be asked to return to the clinic for all remaining study visits for the double-blind treatment period and the follow-up period. For the purpose of efficacy analyses, patients who receive rescue treatment during the study will be considered treatment failures.

### Outcomes Assessed

The primary endpoint is proportion of patients achieving peak esophageal intraepithelial eosinophil count of ≥6 eos/hpf (400×) at week 16.

The key secondary endpoints are: proportion of patients achieving peak esophageal intraepithelial eosinophil count of <15 eos/hpf at week 16; percent change in peak esophageal intraepithelial eosinophil count (eos/hpf) from baseline to week 16; absolute change in mean EoE grade score from the EoE-HSS from baseline to week 16; absolute change in mean EoE stage score from the EoE-HSS from baseline to week 16; absolute change in EoE EREFS from baseline to week 16; change from baseline in the type 2 inflammation transcriptional signature score at weeks 16 and 52; change from baseline to week 16 in the proportion of days with 1 or more EoE signs as measured by the Pediatric EoE Sign/Symptom Questionnaire-caregiver version (PESQ-C) (for patients aged≥1 to <12 years); change from baseline to week 16 in the proportion of total segments within a day (night, morning, afternoon, evening) with 1 or more EoE signs as measured by PESQ-C (for patients aged≥1 to <12 years); change from baseline to week 16 in the proportion of days with 1 or more EoE symptoms as measured by PESQ-P (patient version) (for patients aged≥8 to <12 years); change from baseline to week 16 in the proportion of total segments within a day (night, morning, afternoon, evening) with 1 or more EoE symptoms as measured by PESQ-P (for patients aged≥8 to <12 years); change in total score from baseline to week 16 as measured by the PEESSv2.0-caregiver version questionnaire (for patients aged≥1 to <12 years); normalized enrichment scores (NES) for the relative change from baseline to week 16 in the EoE diagnostic panel (EDP) transcriptome signature; NES for the relative change from baseline to week 16 in the type 2 inflammation transcriptome signature.

Other endpoints include safety endpoints, clinical pharmacology endpoints, and exploratory endpoints, such as impact of dupilumab treatment on global impression of change and severity of disease, and impact of dupilumab treatment on changes in weight and growth.

Procedures for assessing efficacy are described below and are also described in WO 2019/028367 and WO 2021/237110, incorporated by reference herein. Pediatric EoE Siqn/Symptom Questionnaire (PESQ): PESQ is a novel 8-item sign/symptom questionnaire that measures the heterogenous symptom experience of children. The PESQ has a patient version (PESQ-P) and caregiver version (PESQ-C). The PESQ-P is a patient-reported outcome measure intended to be completed independently by EoE patients>8 to <12 years of age. The PESQ-P measures occurrence and severity of EoE symptoms and is completed once daily via an electronic diary. Data from a 14-day period preceding the baseline visit and a 14-day period preceding the week 16 visit is used to calculate the proportion of days or total time segments within a day (night, morning, afternoon, evening) with 1 or more EoE symptoms. The PESQ-C is an observer-reported outcome measure intended to be completed independently by caregivers of all pediatric EoE patients in the study. The PESQ-C measures occurrence of signs of EoE and is completed once daily via an electronic diary. Data from a 14-day period preceding the baseline visit and a 14-day period preceding the week 16 visit is used to calculate the proportion of days or total time segments within a day (night, morning, afternoon, evening) with 1 or more EoE signs.

Pediatric EoE Impact Scale (PEIS): The Pediatric EoE Impact Scale (PEIS) has a patient version (PEIS-P) and a caregiver version (PEIS-C). The PEIS-P is a patient-reported outcome measure intended to be completed independently by pediatric EoE patients>8 to <12 years of age. The PEIS-P assesses the impact of EoE on patients' health-related during the past 1 week. The PEIS-C is intended to be completed independently by caregivers of pediatric EoE patients>1 to <12 years of age. The PEIS-C assesses the impact of the pediatric patient's EoE on caregiver anxiety, social and professional activities, activities of daily living, and relationships during the past 1 week.

Global Impression of Change (GIC): The GIC has a patient version (GIC-P), a caregiver version (GIC-C), and a clinician version (GIC-Clin). The GIC-P is a single-item patient-reported outcome measure intended to be completed independently by pediatric EoE patients>8 to <12 years of age. The GIC-P assesses the patient's impression about the overall change (improvement or worsening) in his/her EoE condition since study treatment initiation. The GIC-C is a single-item observer-reported outcome measure intended to be completed independently by caregivers of pediatric EoE patients>1 to <12 years of age. The GIC-C assesses the caregiver's impression about the overall change (improvement or worsening) in the pediatric patient's EoE condition since study treatment initiation. The GIC-Clin is a single-item observer-reported outcome measure intended to be completed independently by study investigator-physician of all pediatric EoE patients in the study. The GIC-Clin assesses the study investigator's/clinician's impression about the overall change (improvement or worsening) in the pediatric patient's EoE condition since study treatment initiation.

Global Impression of Severity (GIS): The GIS has a patient version (GIS-P), a caregiver version (GIS-C), and a clinician version (GIS-Clin). The GIS-P is a single-item patient-reported outcome measure intended to be completed independently by pediatric EoE patients>8 to <12 years of age. The GIS-P assesses the patient's impression about the overall severity of his/her EoE condition during the past 1 week. The GIS-C is a single-item observer-reported outcomes measure intended to be completed independently by caregivers of pediatric EoE patients>1 to <12 years of age. The GIS-C assesses the caregiver's impression about the overall severity of the pediatric patient's EoE condition during the past 1 week. The GIS-Clin is a single-item observer-reported outcomes measure intended to be completed independently by study investigator-physician of all pediatric EoE patients in the study. The GIS-Clin assesses the study investigator's/clinician's impression about the overall severity of the pediatric patient's EoE condition during the past 1 week.

Pediatric Eosinophilic Esophaqitis Symptom Score (PEESS): The Pediatric Eosinophilic Esophagitis Symptom Score (PEESS) version 2.0-caregiver version is a caregiver reported outcomes measure which assesses the frequency and severity of EoE symptoms among pediatric patients. The PEESSv2.0 caregiver version consists of 20 items and has a one-month recall period. The total PEESSv2.0 score ranges from 0 to 100; higher scores indicate greater symptom burden of among pediatric EoE patients.

EoE-EREFS: The EoE esophageal characteristics will be analyzed based on the EoE-EREFS, a validated scoring system for inflammatory and remodeling features of disease using both overall scores and scores for each individual characteristic (Hirano, 2013). The proximal and distal esophageal regions are scored separately; the score for each region ranges from 0 to 9 and the overall score ranges from 0 to 18. The major esophageal features include: edema (absent, present); rings (absent, mild, moderate, severe); exudates (absent, mild, severe); furrows (absent, mild, severe); and stricture (absent, present). In addition to these major features, data for the following minor features are also captured by the physician performing the endoscopy procedure: crepe paper esophagus (mucosal fragility or laceration upon passage of diagnostic endoscope): absent, present; narrow caliber esophagus (reduced luminal diameter of the majority of the tubular esophagus): absent, present; and stricture diameter. Mucosal changes associated with gastroesophageal reflux disease also are recorded using the Los Angeles classification system for erosions (No Erosions or Grade A, B, C, or D).

Biopsies: Biopsies are obtained by endoscopy at the second screening visit (visit 2), week 16 and week 52 visits, and immediately prior to start of rescue medication or procedures during the double-blind treatment period. A total of 9 mucosal pinch biopsies are collected at each time point from 3 esophageal regions: 3 proximal, 3 mid, and 3 distal. Two samples from each region are used for the histology (needed for study inclusion criteria, as well as endpoint assessment) and the others for RNA extraction or exploratory research. To participate in the study, patients must have a peak intraepithelial eosinophil count 15 eos/hpf (400×) in at least 2 of the 3 esophageal regions sampled. In addition, biopsy specimens from the stomach and/or duodenum are obtained at visit 2 in all patients to rule out alternate etiologies of esophageal eosinophilia. Biopsy samples for histopathological analyses are assessed for peak eosinophil per hpf and EoE Grade Scores and Stage Scores are assigned. EoE Grade and Stage Scores evaluate eight features: eosinophil density, basal zone hyperplasia, eosinophil abscesses, eosinophil surface layering, dilated intercellular spaces, surface epithelial alteration, dyskeratotic epithelial cells, and lamina propria fibrosis (absent/present).

### Pharmacokinetic and Exploratory Biomarker Procedures

In this study, research assessments will be performed to explore EoE, how dupilumab may modify the underlying disease process in EoE, type 2 inflammation, and predictors of dupilumab safety and efficacy. Samples may also be used to evaluate markers related to toxicity, if needed. Samples for total IgE, serum, and eotaxin-3 (heparinized plasma) will be collected at specified time points. The biomarkers studied are relevant to the pathophysiology of EoE, response to treatment (i.e., assessment of type 2 inflammation), and dupilumab mechanism of action.

### EoE Diagnostic Panel and Type 2 Inflammation Transcriptomics

The differential gene expression profiles of esophageal biopsies of EoE patients compared to healthy controls is the EoE disease transcriptome (Sherrill et al., Genes Immun 2014, 15(6):361-369). This disease gene expression signature has been further refined to a smaller gene set to be used as an EoE diagnostic panel (EDP) (Dellon et al., Clin Transl Gastroenterol 2017, 8(2):e74). A gene signature representing type 2 inflammation has been curated from the literature, pre-clinical experiments performed at Regeneron, and dupilumab response signatures from atopic dermatitis and a phase 2 study of EoE.

Normalized Enrichment Score (NES) reflects the degree to which the activity level of a set of transcripts is overrepresented at the extremes (top or bottom) of the entire ranked list of transcripts within a sample and is normalized by accounting for the number of transcripts in the set (Subramanian, 2005; Barbie, 2009). NES scores are calculated for each transcriptome signature for each sample.

### Example 2: Results from Clinical Trial Part A

Part A of the study was a double-blind 16-week treatment period in which patients were randomized 1:1:1: to receive placebo, higher exposure dupilumab, or lower exposure dupilumab according to the tiered, weight-based dosing schema based on body weight at baseline disclosed above. A total of 102 patients were randomized (34 to placebo, 31 to lower exposure dupilumab, and 37 to higher exposure dupilumab). Of these patients, nearly all completed Week 16 (32/34 (94.1%) for placebo; 29/31 (93.5%) for lower exposure dupilumab; 37/37 (100%) for higher exposure dupilumab; 98/102 (96.1%) in total).

Baseline demographics are summarized in Table 1. Baseline demographics were generally comparable across treatment groups. Baseline disease characteristics are summarized in Table 2. In general, patients were highly symptomatic with a high percentage (about 80%) having prior swallowed topical corticosteroid use and about 50% of patients on PPI at randomization. Patients in the higher exposure dupilumab group on average had a higher peak eosinophil count but were less symptomatic by PESQ-C score as compared to other groups. As shown in Table 3, there was a high incidence of atopic co-morbidities in the patient population, underscoring the common Type 2 pathophysiology behind these diseases.

TABLE 1
Baseline Demographics

| | | Lower | Higher | |
|---|---|---|---|---|
| | | Exposure | Exposure | |
| | Placebo | Dupilumab | Dupilumab | Total |
| | (n = 34) | (n = 31) | (n = 37) | (n = 102) |
| Age (years), mean (SD) | 7.2 (3.03) | 7.2 (3.07) | 6.8 (3.11) | 7.1 (3.05) |
| ≥1 = <8 years | 17 (50.0%) | 12 (38.7%) | 20 (54.1%) | 49 (48.0%) |
| ≥8 to <12 years | 17 (50.0%) | 19 (61.3%) | 17 (45.9%) | 53 (52.0%) |
| Sex (male), n (%) | 25 (73.5%) | 25 (80.6%) | 28 (75.7%) | 78 (76.5%) |
| Race, n (%) | | | | |
| White | 30 (88.2%) | 22 (71.0%) | 32 (86.5%) | 84 (82.4%) |
| Black or African American | 3 (8.8%) | 4 (12.9%) | 4 (10.8%) | 11 (10.8%) |
| Asian | 0 | 1 (3.2%) | 1 (2.7%) | 2 (2.0%) |
| Other | 1 (2.9%) | 4 (12.9%) | 0 | 5 (4.9%) |
| Weight (kg), mean (SD) | 28.3 (11.99) | 26.1 (10.06) | 26.0 (10.60) | 26.8 (10.87) |
| BMI, mean (SD) | 17.3 (2.88) | 16.6 (2.79) | 17.3 (2.89) | 17.1 (2.84) |
| Region, n (%) | | | | |
| North America | 34 (100%) | 31 (100%) | 37 (100%) | 102 (100%) |

TABLE 2
Baseline Disease Characteristics

| | | Lower | Higher | |
|---|---|---|---|---|
| | | Exposure | Exposure | |
| | Placebo | Dupilumab | Dupilumab | Total |
| | (n = 34) | (n = 31) | (n = 37) | (n = 102) |
| Duration of EoE (years), | 4.40 (2.793) | 4.14 (2.894) | 3.79 (2.900) | 4.10 (2.846) |
| mean (SD) | | | | |
| Duration of EoE (years) | | | | |
| Group, n (%) | | | | |
| <5 years | 21 (61.8%) | 19 (61.3%) | 25 (67.6%) | 65 (63.7%) |
| ≥5 years | 13 (38.2%) | 12 (38.7%) | 12 (32.4%) | 37 (36.3%) |
| PPI at randomization, n (%) | 10 (29.4%) | 19 (61.3%) | 20 (54.1%) | 49 (48.0%) |
| History of prior esophageal | 0 | 2 (6.5%) | 0 | 2 (2.9%) |
| dilations, n (%) | | | | |
| Prior swallowed topical | 27 (79.4%) | 27 (87.1%) | 28 (75.7%) | 82 (80.4%) |
| steroid, n (%) | | | | |
| Topical steroid effective, n (%) | 14 (41.2%) | 7 (22.6%) | 9 (24.3%) | 30 (29.4%) |
| Inadequate response, intolerant, | 15 (44.1%) | 21 (67.7%) | 23 (62.2%) | 59 (57.8%) |
| and/or contraindicated to swallowed | | | | |
| topical steroids, n (%) | | | | |
| Peak Eos count of 3 regions, | 81.8 (36.14) | 74.7 (30.56) | 92.0 (48.04) | 83.3 (39.72) |
| mean (SD) | | | | |
| Mean Eos count of 3 regions, | 57.4 (21.90) | 55.9 (24.67) | 69.3 (38.16) | 61.3 (29.90) |
| mean (SD) | | | | |
| EoE Grade Score [0-3], | 1.3 (0.36) | 1.2 (0.33) | 1.3 (0.41) | 1.3 (0.37) |
| mean (SD) | | | | |
| EoE Stage Score [0-3], | 1.3 (0.36) | 1.2 (0.31) | 1.3 (0.32) | 1.3 (0.33) |
| mean (SD) | | | | |
| EREFS Score [0-18], | 7.3 (2.51) | 6.4 (2.72) | 6.8 (2.66) | 6.9 (2.62) |
| mean (SD) | | | | |
| PESQ-C (Proportion of Days | 0.53 (0.361) | 0.64 (0.368) | 0.46 (0.378) | 0.54 (0.373) |
| with 1 or more EoE Signs) | | | | |
| [0-1], mean (SD) | | | | |
| PESQ-C (Number of Sign-Free | 6.5 (5.06) | 5.1 (5.15) | 7.6 (5.30) | 6.5 (5.22) |
| Days) [0-14], mean (SD) | | | | |

TABLE 3
Concurrent Atopic/Allergic Conditions

| | | Lower | Higher | |
|---|---|---|---|---|
| | | Exposure | Exposure | |
| | Placebo | Dupilumab | Dupilumab | Total |
| | (n = 34) | (n = 31) | (n = 37) | (n = 102) |
| Number of patients with ≥1 | 32 (94.1%) | 31 (100%) | 37 (100%) | 100 (98%) |
| concurrent allergic conditions | | | | |
| Allergic Rhinitis, n (%) | 22 (64.7%) | 27 (87.1%) | 29 (78.4%) | 78 (76.5%) |
| Asthma, n (%) | 14 (41.2%) | 18 (58.1%) | 24 (64.9%) | 56 (54.9%) |
| Food Allergy, n (%) | 27 (79.4%) | 25 (80.6%) | 31 (83.8%) | 83 (81.4%) |
| Atopic Dermatitis, n (%) | 19 (55.9%) | 15 (48.4%) | 21 (56.8%) | 55 (53.9%) |
| Allergic Conjunctivitis, n (%) | 4 (11.8%) | 5 (16.1%) | 4 (10.8%) | 13 (12.7%) |
| Peripheral blood eosinophils | 0.66 (0.407) | 0.62 (0.353) | 0.81 (0.604) | 0.70 (0.478) |
| (Giga/L), mean (SD) | | | | |
| Peripheral blood eosinophils | 0.62 | 0.55 | 0.65 | 0.61 |
| (Giga/L), median (Q1:Q3) | (0.41:0.84) | (0.33:0.75) | (0.40:1.08) | (0.39:0.84) |
| Serum total IgE (IU/L), | 501.6 | 858.1 | 1725.0 | 1054.5 |
| mean (SD) | (567.46) | (1387.59) | (3372.30) | (2239.74) |
| Serum total IgE (IU/L), | 400.0 | 392.0 | 521.5 | 400.0 |
| median (Q1:Q3) | (66.3:642.0) | (108.0:973.0) | (164.5:1535.5) | (112.0:922.0) |

### Efficacy

The efficacy results from Part A for the primary and secondary endpoints are shown in Table 4 below. Both dupilumab exposure regimens (higher exposure dupilumab and lower exposure dupilumab) demonstrated significant improvements in achieving histological remission. The primary endpoint assessed the proportion of patients achieving peak eosinophil count of 56 eos/hpf at Week 16. While only 2.9% of placebo patients (n=1/34) achieved≥6 eos/hpf at Week 16, 67.6% of patients in the higher exposure dupilumab regimen (n=25/37) and 55.1% of patients in the lower exposure dupilumab regimen (n=18/31) achieved≥6 eos/hpf at Week 16.

The higher exposure dupilumab regimen met all pre-specified secondary efficacy endpoints for histology, endoscopy, and transcriptome (all p-values<0.0001) (Table 4). Children receiving the higher exposure dupilumab regimen experienced the following changes at Week 16:
86% reduction in peak esophageal intraepithelial eosinophil count from baseline compared to 21% increase for placebo (p<0.0001)
0.88 and 0.84 reduction in disease severity and extent of disease, respectively, at the microscopic level from baseline [as measured by EoEHSS Mean Grade Score and Mean Stage Score, respectively] compared to 0.02 and 0.05 increase for placebo (both p<0.0001)
3.5-point reduction in abnormal endoscopic findings from baseline [as measured by EoE-EREFS score] compared to 0.3-point increase for placebo (p<0.0001)

The higher exposure dupilumab regimen also demonstrated improvements over placebo in the secondary symptom endpoint of absolute change (LS Mean) from baseline to Week 16 in the proportion of days with one or more EoE signs as measured by PESQ-C, although it did not achieve statistical significance (placebo: −0.17; higher exposure dupilumab regimen: −0.28; Δ−0.10 (p=0.15); which equates to approximately 1.5 fewer days with EoE signs vs placebo). The lower exposure dupilumab regimen was comparable or numerically lower than higher exposure dupilumab on the pre-specified secondary efficacy endpoints (histology, endoscopy, transcriptome, and symptoms, as shown in Table 4. Additionally, the higher exposure dupilumab regimen demonstrated greater improvements than lower exposure dupilumab regimen in other patient-reported outcomes, global impression, and quality of life.

TABLE 4
Efficacy Results for Primary and Secondary Endpoints

| | | | Dupilumab | Dupilumab | Δ Dupi High vs | Δ Dupi Low vs |
|---|---|---|---|---|---|---|
| | | Placebo | High | Low | Pbo | Pbo |
| | Endpoint | (n = 34) | (n = 37) | (n = 31) | (95% CI) | (95% CI) |
| Primary | Proportion of patients achieving peak esophageal | 1 | 25 | 18 | 64.5* | 55.1* |
| | intraepithelial eosinophil ≤6 eos/hpf, n (%) | (2.9%) | (67.6%) | (58.1%) | (48.19, 80.85) | (36.85, 73.42) |
| Secondary | Proportion of patients achieving peak esophageal | 1 | 31 | 21 | 81* | 65.4** |
| | intraepithelial eosinophil ≤15 eos/hpf, n (%) | (2.9%) | (83.8%) | (67.7%) | (68.07, 94.10) | (48.02, 82.81) |
| | Percent change in peak esophageal intraepithelial | 20.98% | −86.09 | −77.93 | −107.07* | −98.92** |
| | eosinophil count (eos/hpf) from BL, LE mean (SE) | (12.232) | (11.841) | (12.893) | (−139.249, −74.900) | (−132.463, −65.370) |
| | Absolute change in Mean Grade Score (0-3) from the | 0.023 | −0.879 | −0.757 | −0.902* | −0.780** |
| | Histology Scoring System (EoEHSS) from BL, LS | (0.0498) | (0.0481) | (0.0524) | (−1.0325, −0.7714) | (−0.9170, −0.6440) |
| | mean (SE) | | | | | |
| | Absolute change in Mean stage Score (0-3) from the | 0.048 | −0.835 | −0.721 | −0.883* | −0.769** |
| | EoEHSS from BL, LS mean (SE) | (0.0482) | (0.0466) | (0.0507) | (−1.0095, −0.7568) | (−0.9013, −0.6362) |
| | Normalized Enrichment Score (NES) of the relative | 0.340 | −1.895 | −1.930 | −2.220* | −2.190 |
| | change from BL in the type 2 inflammation signature, | | | | (−2.4400, −1.9500) | (−2.4500, −1.8200) |
| | median | | | | | |
| | NES for the relative change from BL in the EoE | 0.180 | −2.630 | −2.710 | −2.840* | −2.700 |
| | diagnostic panel | | | | (−3.3500, −1.9600) | (−3.3100, −1.6200) |
| | Absolute change in EoE-Endoscopic Reference Score | 0.3 | −3.5 | −3.0 | −3.8* | −3.3** |
| | (EoE-EREFS) (0-18) from BL, LS mean (SE) | (0.45) | (0.42) | (0.48) | (−4.94, −2.63) | (−4.59, −2.10) |
| | Absolute change from BL to week 16 in the | −0.17 | −0.28 | −0.18 | −0.10^{x} | 0.00^{x} |
| | proportion of days with 1 or more EoE signs as | (0.054) | (0.052) | (0.060) | (−0.244, 0.038) | (−0.155, 0.146) |
| | measured by PESQ-C (−1 to 1), LS mean (SE) | | | | | |

BL = baseline
*p < 0.0001;
**nominal p < 0.0001;
^{x}p = N.S. (not significant)

### Body Weight

An exploratory endpoint was the change from baseline in body weight for age percentile at Week 16. Both dupilumab-treated groups demonstrated greater changes from baseline in body weight for age percentile at Week 16 versus placebo (placebo: mean change of 0.29; higher exposure dupilumab: mean change of 3.09; for lower exposure dupilumab: mean change of 2.88).

### Safety

Both the higher exposure and lower exposure dupilumab regimens were well tolerated and demonstrated an acceptable safety profile, with no new safety concerns identified. Safety results were generally consistent with the known safety profile of Dupixent in its approved EoE indication for children and adults 12 years and older. For the 16-week treatment period, overall rates of adverse events (AEs) were 79% for Dupixent and 91% for placebo. AEs more commonly (≥5%) observed with Dupixent compared to placebo included COVID-19 (21% Dupixent, 0% placebo; all cases were mild or moderate and did not lead to treatment discontinuation), rash (9% Dupixent, 6% placebo), headache (8% Dupixent, 3% placebo), viral gastroenteritis (6% Dupixent, 3% placebo), diarrhea (6% Dupixent, 3% placebo) and nausea (6% Dupixent, 0% placebo). Rates of treatment discontinuation due to AEs prior to week 16 were 0% for Dupixent and 6% for placebo.

No use of rescue treatment was needed in any of the placebo, lower exposure dupilumab, or higher exposure dupilumab groups.

### Conclusion

In Part A of this two-part study to assess efficacy and safety of dupilumab in patients with EoE aged≥1 to <12 years of age, treatment with either higher exposure dupilumab or lower exposure dupilumab according to a tiered, weight-based dosing schema based on body weight at baseline met the primary histologic endpoint at Week 16. Higher exposure dupilumab met all secondary efficacy endpoints for histology, endoscopy, and transcriptome. Pediatric higher exposure dupilumab treatment resulted in numerically higher or comparable results to 300 mg QW dosing in adults and adolescents for histological, endoscopic, and transcriptomic efficacy endpoints. Dupilumab was well tolerated and demonstrated a similar safety profile to the known safety profile of dupilumab in adults and adolescents with EoE.

### Example 3: Results from Clinical Trial Part B

Part B of the study was an open-label 36-week extended treatment period for patients who participated in Part A. A total of 98 patients (out of 102) from Part A enrolled in Part B. All patients were made aware that they were on active treatment in Part B, but were blinded to their dosing regimen. The treatment groups for Part B are shown in Table 5 below:

TABLE 5
Treatment Groups for Part B (Extended Active Treatment Period Over 36 Weeks)

| | ≥5 to <15 kg | ≥15 to <30 kg | ≥30 to <60 kg | ≥60 kg |
|---|---|---|---|---|
| Higher Exposure | 100 mg Q2W | 200 mg Q2W | 300 mg Q2W | 300 mg QW |
| Dupilumab | | | | |
| (“dupi high”) | | | | |
| Lower Exposure | 200 mg Q4W | 300 mg Q4W | 200 mg Q2W | 300 mg Q2W |
| Dupilumab | | | | |
| (“dupi low”) | | | | |

### Efficacy

As shown in Table 6 below, it was found that dupilumab treatment effects on histological, endoscopic, symptoms, and transcriptome effects observed at Week 16 were sustained through Week 52. When placebo-treated patients from Part A were switched to dupilumab treatment in Part B, the efficacy was generally similar to that observed for the dupilumab-treated patients in Part A.

TABLE 6
Efficacy Results for Primary and Secondary Endpoints

| | Endpoints at Week 52 | | | |
|---|---|---|---|---|
| | Pbo/ | Pbo/ | Dupi low/ | Dupi high/ |
| | dupi | dupi | dupi | dupi |
| | low | high | low | high |
| Efficacy Parameters | (n = 14) | (n = 18) | (n = 39) | (n = 37) |
| Proportion of patients achieving peak | 13/14 | 9/17 | 19/29 | 22/35 |
| esophageal intraepithelial eosinophil ≤6 | (92.9%) | (52.9%) | (65.5%) | (62.9%) |
| eos/hpf, n (%) | | | | |
| Proportion of patients achieving peak | 13/14 | 11/17 | 20/29 | 30/35 |
| esophageal intraepithelial eosinophil ≤15 | (92.9%) | (64.7%) | (69.0%) | (85.7%) |
| eos/hpf, n (%) | | | | |
| Percent change in peak esophageal | −92.72% | −76.83% | −85.41% | −90.97% |
| intraepithelial eosinophil count | (19.229) | (41.228) | (22.851) | (14.482) |
| (eos/hpf) from Part A BL, LE mean (SE) | | | | |
| Absolute change in Mean Grade Score | −0.804 | −0.885 | −0.773 | −0.967 |
| (0-3) from the Histology Scoring System | (0.3099) | (0.2962) | (0.3374) | (0.3920) |
| (EoEHSS) from Part A BL, LS mean (SE) | | | | |
| Absolute change in Mean stage Score | −0.767 | −0.885 | −0.784 | −0.892 |
| (0-3) from the EoEHSS from Part A BL, | (0.3114) | (0.3485) | (0.3183) | (0.3181) |
| LS mean (SE) | | | | |
| Normalized Enrichment Score (NES) for | −1.960 | −1.965 | −1.920 | −1.920 |
| the relative change from Part A BL in | (n = 8) | (n = 12) | (n = 16) | (n = 21) |
| the type 2 inflammation signature, | | | | |
| median | | | | |
| NES for the relative change from Part A | −2.715 | −2.615 | −2.625 | −2.670 |
| BL in the EoE diagnostic panel, median | (n = 8) | (n = 12) | (n = 16) | (n = 21) |
| Absolute change in EoE-Endoscopic | −5.82 | −3.64 | −4.50 | −4.77 |
| Reference Score (EoE-EREFS) (0-18) | (1.722) | (3.342) | (3.203) | (3.081) |
| from Part A BL, LS mean (SE) | (n = 11) | (n = 14) | (n = 22) | (n = 30) |
| Absolute change from Part A BL in the | −0.20 | −0.47 | −0.49 | −0.30 |
| proportion of days with 1 or more EoE | (0.373) | (0.395) | (0.339) | (0.299) |
| signs as measured by PESQ-C (−1 to 1), | (n = 6) | (n = 9) | (n = 18) | (n = 27) |
| LS mean (SE) | | | | |

Part A BL = Part A baseline

### Safety

Overall incidences of TEAEs were comparable across the treatment arms, with the majority being mild or moderate in intensity. There was a low incidence of SAEs and AEs leading to treatment discontinuation. All SAEs were deemed unrelated to the study drug.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

TABLE 7
Informal Sequence Listing

| SEQ | | |
|---|---|---|
| ID | | |
| NO | Sequence | Description |
| 1 | EVQLVESGGGLEQPGGSLRLSCAGSGFTFRDYAMTWVRQAPGKGLEWVSSISGSG | Dupilumab HCVR |
| | GNTYYADSVKGRFTISRDNSKNTLYLQMNSLRAEDTAVYYCAKDRLSITIRPRYYGLD | amino acid |
| | VWGQGTTVTVS | sequence |
| 2 | DIVMTQSPLSLPVTPGEPASISCRSSQSLLYSIGYNYLDWYLQKSGQSPQLLIYLGSNR | Dupilumab LCVR |
| | ASGVPDRFSGSGSGTDFTLKISRVEAEDVGFYYCMQALQTPYTFGQGTKLEIK | amino acid |
| | | sequence |
| 3 | GFTFRDYA | Dupilumab HCDR1 |
| | | amino acid |
| | | sequence |
| 4 | ISGSGGNT | Dupilumab HCDR2 |
| | | amino acid |
| | | sequence |
| 5 | AKDRLSITIRPRYYGLDV | Dupilumab HCDR3 |
| | | amino acid |
| | | sequence |
| 6 | QSLLYSIGYNY | Dupilumab LCDR1 |
| | | amino acid |
| | | sequence |
| | LGS | Dupilumab LCDR2 |
| | | amino acid |
| | | sequence |
| 8 | MQALQTPYT | Dupilumab LCDR3 |
| | | amino acid |
| | | sequence |
| 9 | EVQLVESGGGLEQPGGSLRLSCAGSGFTFRDYAMTWVRQAPGKGLEWVSSISGSG | Dupilumab heavy |
| | GNTYYADSVKGRFTISRDNSKNTLYLQMNSLRAEDTAVYYCAKDRLSITIRPRYYGLD | chain |
| | VWGQGTTVTVSSASTKGPSVFPLAPCSRSTSESTAALGCLVKDYFPEPVTVSWNSGA | amino acid |
| | LTSGVHTFPAVLQSSGLYSLSSVVTVPSSSLGTKTYTCNVDHKPSNTKVDKRVESKYGP | sequence |
| | PCPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLGK | |
| 10 | DIVMTQSPLSLPVTPGEPASISCRSSQSLLYSIGYNYLDWYLQKSGQSPQLLIYLGSNR | Dupilumab light |
| | ASGVPDRFSGSGSGTDFTLKISRVEAEDVGFYYCMQALQTPYTFGQGTKLEIKRTVAA | chain |
| | PSVFIFPPSDEQLKSGTASVVCLLNNFYPREAKVQWKVDNALQSGNSQESVTEQDSK | amino acid |
| | DSTYSLSSTLTLSKADYEKHKVYACEVTHQGLSSPVTKSFNRGEC | sequence |
| 11 | MKVLQEPTCVSDYMSISTCEWKMNGPTNCSTELRLLYQLVFLLSEAHTCIPENNGGA | Human IL-4Rα |
| | GCVCHLLMDDVVSADNYTLDLWAGQQLLWKGSFKPSEHVKPRAPGNLTVHTNVS | |
| | DTLLLTWSNPYPPDNYLYNHLTYAVNIWSENDPADFRIYNVTYLEPSLRIAASTLKSGI | |
| | SYRARVRAWAQCYNTTWSEWSPSTKWHNSYREPFEQH | |
| 12 | EIVLTQSPGTLSLSPGERATLSCRASQSVSNSYLAWYQQKPGQAPRLLIFGASSRATGI | SCB-VL-39 |
| | PDRFSGSGSGTDFTLTISRLEPEDFAVYYCQQYGSSPPWTFGQGTKVEIK | |
| 13 | EIVLTQSPGTLSLSPGERATLSCRASQSVSSSYLAWYQQKPGQAPRLLIYGASSRATGI | SCB-VL-40 |
| | PDRFSGSGSGTDFTLTISRLEPEDFAVYYCQQYGSSPPWTFGQGTKVEIK | |
| 14 | EIVLTQSPGTLSLSPGERATLSCRASQSVSSSYLAWYQQKPGQAPRLLIFGASSRAPGI | SCB-VL-41 |
| | PDRFSGSGSGTDFTLTISRLEPEDFAVYYCQQYGSSPPWTFGQGTKVEIK | |
| 15 | EIVLTQSPGTLSLSPGERATLSCRASQSVSNSYLAWYQQKPGQAPRLLIYGASSRATGI | SCB-VL-42 |
| | PDRFSGSGSGTDFTLTISRLEPEDFAVYYCQQYGSSPPWTFGQGTKVEIK | |
| 16 | EIVLTQSPGTLSLSPGERATLSCRASQSVSNSYLAWYQQKPGQAPRLLIFGASSRAPGI | SCB-VL-43 |
| | PDRFSGSGSGTDFTLTISRLEPEDFAVYYCQQYGSSPPWTFGQGTKVEIK | |
| 17 | EIVLTQSPGTLSLSPGERATLSCRASQSVSSSYLAWYQQKPGQAPRLLIYGASSRAPGI | SCB-VL-44 |
| | PDRFSGSGSGTDFTLTISRLEPEDFAVYYCQQYGSSPPWTFGQGTKVEIK | |
| 18 | EIVLTQSPGTLSLSPGERATLSCRASQSVSSSYLAWYQQKPGQAPRLLIFGASSRATGI | SCB-VL-45 |
| | PDRFSGSGSGTDFTLTISRLEPEDFAVYYCQQYDHSPPWTFGQGTKVEIK | |
| 19 | EIVLTQSPGTLSLSPGERATLSCRASQSVSSSYLAWYQQKPGQAPRLLIFGASSRATGI | SCB-VL-46 |
| | PDRFSGSGSGTDFTLTISRLEPEDFAVYYCQQYGSSAGWTFGQGTKVEIK | |
| 20 | EIVLTQSPGTLSLSPGERATLSCRASQSVSSSYLAWYQQKPGQAPRLLIFGASSRATGI | SCB-VL-47 |
| | PDRFSGSGSGTDFTLTISRLEPEDFAVYYCQQYDHSAGWTFGQGTKVEIK | |
| 21 | EIVLTQSPGTLSLSPGERATLSCRASQSVSNSYLAWYQQKPGQAPRLLIFGASSRATGI | SCB-VL-48 |
| | PDRFSGSGSGTDFTLTISRLEPEDFAVYYCQQYDHSPPWTFGQGTKVEIK | |
| 22 | EIVLTQSPGTLSLSPGERATLSCRASQSVSSSYLAWYQQKPGQAPRLLIYGASSRATGI | SCB-VL-49 |
| | PDRFSGSGSGTDFTLTISRLEPEDFAVYYCQQYDHSPPWTFGQGTKVEIK | |
| 23 | EIVLTQSPGTLSLSPGERATLSCRASQSVSSSYLAWYQQKPGQAPRLLIFGASSRAPGI | SCB-VL-50 |
| | PDRFSGSGSGTDFTLTISRLEPEDFAVYYCQQYDHSPPWTFGQGTKVEIK | |
| 24 | EIVLTQSPGTLSLSPGERATLSCRASQSVSSSYLAWYQQKPGQAPRLLIYGASSRAPGI | SCB-VL-51 |
| | PDRFSGSGSGTDFTLTISRLEPEDFAVYYCQQYDHSAGWTFGQGTKVEIK | |
| 25 | EIVLTQSPGTLSLSPGERATLSCRASQSVSNSYLAWYQQKPGQAPRLLIFGASSRAPGI | SCB-VL-52 |
| | PDRFSGSGSGTDFTLTISRLEPEDFAVYYCQQYDHSAGWTFGQGTKVEIK | |
| 26 | EIVLTQSPGTLSLSPGERATLSCRASQSVSNSYLAWYQQKPGQAPRLLIYGASSRATGI | SCB-VL-53 |
| | PDRFSGSGSGTDFTLTISRLEPEDFAVYYCQQYDHSAGWTFGQGTKVEIK | |
| 27 | EIVLTQSPGTLSLSPGERATLSCRASQSVSSSYLAWYQQKPGQAPRLLIFGASSRAPGI | SCB-VL-54 |
| | PDRFSGSGSGTDFTLTISRLEPEDFAVYYCQQYDHSAGWTFGQGTKVEIK | |
| 28 | EIVLTQSPGTLSLSPGERATLSCRASQSVSSSYLAWYQQKPGQAPRLLIYGASSRATGI | SCB-VL-55 |
| | PDRFSGSGSGTDFTLTISRLEPEDFAVYYCQQYDHSAGWTFGQGTKVEIK | |
| 29 | EIVLTQSPGTLSLSPGERATLSCRASQSVSNSYLAWYQQKPGQAPRLLIFGASSRATGI | SCB-VL-56 |
| | PDRFSGSGSGTDFTLTISRLEPEDFAVYYCQQYDHSAGWTFGQGTKVEIK | |
| 30 | EIVLTQSPGTLSLSPGERATLSCRASQSVSSSYLAWYQQKPGQAPRLLIFGASSRATGI | SCB-VL-57 |
| | PDRFSGSGSGTDFTLTISRLEPEDFAVYYCQQYGSSPPWTFGQGTKVEIK | |
| 31 | EIVLTQSPGTLSLSPGERATLSCRASQSVSNSYLAWYQQKPGQAPRLLIYGASSRAPGI | SCB-VL-58 |
| | PDRFSGSGSGTDFTLTISRLEPEDFAVYYCQQYDHSAGWTFGQGTKVEIK | |
| 32 | EVQLVESGGGLVHPGGSLRLSCAGSGFTFSRNAMFWVRQAPGKGLEWVSGIGTGG | SCB-VH-59 |
| | ATNYADSVKGRFTISRDNAKNSLYLQMNSLRAEDMAVYYCARGRYYFDYWGQGTL | |
| | VTVSS | |
| 33 | EVQLVQSGGGLVQPGGSLRLSCAGSGFTFSRNAMFWVRQAPGKGLEWVSGIGTG | SCB-VH-60 |
| | GATNYADSVKGRFTISRDNAKNSLYLQMNSLRAEDMAVYYCARGRYYFDYWGQGT | |
| | LVTVSS | |
| 34 | EVQLVQSGGGLVHPGGSLRLSCAASGFTFSRNAMFWVRQAPGKGLEWVSGIGTGG | SCB-VH-61 |
| | ATNYADSVKGRFTISRDNAKNSLYLQMNSLRAEDMAVYYCARGRYYFDYWGQGTL | |
| | VTVSS | |
| 35 | EVQLVQSGGGLVHPGGSLRLSCAGSGFTFSRNAMFWVRQAPGKGLEWVSGIGTGG | SCB-VH-62 |
| | ATSYADSVKGRFTISRDNAKNSLYLQMNSLRAEDMAVYYCARGRYYFDYWGQGTLV | |
| | TVSS | |
| 36 | EVQLVQSGGGLVHPGGSLRLSCAGSGFTFSRNAMFWVRQAPGKGLEWVSGIGTGG | SCB-VH-63 |
| | ATNYADSVKGRFTISRDNAKNSLYLQMNSLRAEDTAVYYCARGRYYFDYWGQGTLV | |
| | TVSS | |
| 37 | EVQLVESGGGLVQPGGSLRLSCAGSGFTFSRNAMFWVRQAPGKGLEWVSGIGTGG | SCB-VH-64 |
| | ATNYADSVKGRFTISRDNAKNSLYLQMNSLRAEDMAVYYCARGRYYFDYWGQGTL | |
| | VTVSS | |
| 38 | EVQLVESGGGLVHPGGSLRLSCAASGFTFSRNAMFWVRQAPGKGLEWVSGIGTGG | SCB-VH-65 |
| | ATNYADSVKGRFTISRDNAKNSLYLQMNSLRAEDMAVYYCARGRYYFDYWGQGTL | |
| | VTVSS | |
| 39 | EVQLVQSGGGLVQPGGSLRLSCAASGFTFSRNAMFWVRQAPGKGLEWVSGIGTGG | SCB-VH-66 |
| | ATNYADSVKGRFTISRDNAKNSLYLQMNSLRAEDMAVYYCARGRYYFDYWGQGTL | |
| | VTVSS | |
| 40 | EVQLVQSGGGLVHPGGSLRLSCAGSGFTFSRNAMFWVRQAPGKGLEWVSGIGTGG | SCB-VH-67 |
| | ATSYADSVKGRFTISRDNAKNSLYLQMNSLRAEDTAVYYCARGRYYFDYWGQGTLV | |
| | TVSS | |
| 41 | EVQLVQSGGGLVHPGGSLRLSCAGSGFTFSRNAMFWVRQAPGKGLEWVSGIGTGG | SCB-VH-68 |
| | ATNYADSVKGRFTISRDNAKNSLYLQMNSLRAEDMAVYYCARGRYYFPWWGQGTL | |
| | VTVSS | |
| 42 | EVQLVESGGGLVHPGGSLRLSCAGSGFTFSRNAMFWVRQAPGKGLEWVSGIGTGG | SCB-VH-69 |
| | ATNYADSVKGRFTISRDNAKNSLYLQMNSLRAEDMAVYYCARGRYYFPWWGQGTL | |
| | VTVSS | |
| 43 | EVQLVQSGGGLVQPGGSLRLSCAGSGFTFSRNAMFWVRQAPGKGLEWVSGIGTG | SCB-VH-70 |
| | GATNYADSVKGRFTISRDNAKNSLYLQMNSLRAEDMAVYYCARGRYYFPWWGQG | |
| | TLVTVSS | |
| 44 | EVQLVQSGGGLVHPGGSLRLSCAASGFTFSRNAMFWVRQAPGKGLEWVSGIGTGG | SCB-VH-71 |
| | ATNYADSVKGRFTISRDNAKNSLYLQMNSLRAEDMAVYYCARGRYYFPWWGQGTL | |
| | VTVSS | |
| 45 | EVQLVQSGGGLVHPGGSLRLSCAGSGFTFSRNAMFWVRQAPGKGLEWVSGIGTGG | SCB-VH-72 |
| | ATSYADSVKGRFTISRDNAKNSLYLQMNSLRAEDMAVYYCARGRYYFPWWGQGTL | |
| | VTVSS | |
| 46 | EVQLVQSGGGLVHPGGSLRLSCAGSGFTFSRNAMFWVRQAPGKGLEWVSGIGTGG | SCB-VH-73 |
| | ATNYADSVKGRFTISRDNAKNSLYLQMNSLRAEDTAVYYCARGRYYFPWWGQGTL | |
| | VTVSS | |
| 47 | EVQLVQSGGGLVHPGRSLRLSCAGSGFTFSRNAMFWVRQAPGKGLEWVSGIGTGG | SCB-VH-74 |
| | ATNYADSVKGRFTISRDNAKNSLYLQMNSLRAEDMAVYYCARGRYYFDYWGQGTL | |
| | VTVSS | |
| 48 | EVQLVQSGGGLVHPGGSLRLTCAGSGFTFSRNAMFWVRQAPGKGLEWVSGIGTG | SCB-VH-75 |
| | GATNYADSVKGRFTISRDNAKNSLYLQMNSLRAEDMAVYYCARGRYYFDYWGQGT | |
| | LVTVSS | |
| 49 | EVQLVQSGGGLVHPGGSLRLSCAGSGFTFSRNAMHWVRQAPGKGLEWVSGIGTG | SCB-VH-76 |
| | GATNYADSVKGRFTISRDNAKNSLYLQMNSLRAEDMAVYYCARGRYYFDYWGQGT | |
| | LVTVSS | |
| 50 | EVQLVQSGGGLVHPGGSLRLSCAGSGFTFSRNAMFWVRQAPGEGLEWVSGIGTGG | SCB-VH-77 |
| | ATNYADSVKGRFTISRDNAKNSLYLQMNSLRAEDMAVYYCARGRYYFDYWGQGTL | |
| | VTVSS | |
| 51 | EVQLVQSGGGLVHPGGSLRLSCAGSGFTFSRNAMFWVRQAPGKGLEWVSGIGTGG | SCB-VH-78 |
| | ATNYADSVKGRFTISRDEAKNSLYLQMNSLRAEDMAVYYCARGRYYFDYWGQGTLV | |
| | TVSS | |
| 52 | EVQLVQSGGGLVHPGGSLRLSCAGSGFTFSRNAMFWVRQAPGKGLEWVSGIGTGG | SCB-VH-79 |
| | ATNYADSVKGRFTISRDNAKNSLYLQMNSLRAGDMAVYYCARGRYYFDYWGQGTL | |
| | VTVSS | |
| 53 | EVQLVQSGGGLVHPGGSLRLSCAGSGFTFDDYAMFWVRQAPGKGLEWVSGIGTG | SCB-VH-80 |
| | GATNYADSVKGRFTISRDNAKNSLYLQMNSLRAEDMAVYYCARGRYYFDYWGQGT | |
| | LVTVSS | |
| 54 | EVQLVQSGGGLVQPGGSLRLSCAASGFTFSRNAMFWVRQAPGKGLEWVSGIGTGG | SCB-VH-81 |
| | ATSYADSVKGRFTISRDNAKNSLYLQMNSLRAEDTAVYYCARGRYYFPWWGQGTLV | |
| | TVSS | |
| 55 | EVQLVESGGGLVHPGGSLRLSCAASGFTFSRNAMFWVRQAPGKGLEWVSGIGTGG | SCB-VH-82 |
| | ATSYADSVKGRFTISRDNAKNSLYLQMNSLRAEDTAVYYCARGRYYFPWWGQGTLV | |
| | TVSS | |
| 56 | EVQLVESGGGLVQPGGSLRLSCAGSGFTFSRNAMFWVRQAPGKGLEWVSGIGTGG | SCB-VH-83 |
| | ATSYADSVKGRFTISRDNAKNSLYLQMNSLRAEDTAVYYCARGRYYFPWWGQGTLV | |
| | TVSS | |
| 57 | EVQLVESGGGLVQPGGSLRLSCAASGFTFSRNAMFWVRQAPGKGLEWVSGIGTGG | SCB-VH-84 |
| | ATNYADSVKGRFTISRDNAKNSLYLQMNSLRAEDTAVYYCARGRYYFPWWGQGTL | |
| | VTVSS | |
| 58 | EVQLVESGGGLVQPGGSLRLSCAASGFTFSRNAMFWVRQAPGKGLEWVSGIGTGG | SCB-VH-85 |
| | ATSYADSVKGRFTISRDNAKNSLYLQMNSLRAEDMAVYYCARGRYYFPWWGQGTL | |
| | VTVSS | |
| 59 | EVQLVQSGGGLVHPGGSLRLSCAASGFTFSRNAMFWVRQAPGKGLEWVSGIGTGG | SCB-VH-86 |
| | ATSYADSVKGRFTISRDNAKNSLYLQMNSLRAEDTAVYYCARGRYYFPWWGQGTLV | |
| | TVSS | |
| 60 | EVQLVQSGGGLVQPGGSLRLSCAGSGFTFSRNAMFWVRQAPGKGLEWVSGIGTG | SCB-VH-87 |
| | GATSYADSVKGRFTISRDNAKNSLYLQMNSLRAEDTAVYYCARGRYYFPWWGQGTL | |
| | VTVSS | |
| 61 | EVQLVESGGGLVHPGGSLRLSCAGSGFTFSRNAMFWVRQAPGKGLEWVSGIGTGG | SCB-VH-88 |
| | ATSYADSVKGRFTISRDNAKNSLYLQMNSLRAEDTAVYYCARGRYYFPWWGQGTLV | |
| | TVSS | |
| 62 | EVQLVQSGGGLVHPGGSLRLSCAGSGFTFSRNAMFWVRQAPGKGLEWVSGIGTGG | SCB-VH-89 |
| | ATSYADSVKGRFTISRDNAKNSLYLQMNSLRAEDTAVYYCARGRYYFPWWGQGTLV | |
| | TVSS | |
| 63 | EVQLVESGGGLVQPGGSLRLSCAASGFTFSRNAMFWVRQAPGKGLEWVSGIGTGG | SCB-VH-90 |
| | ATNYADSVKGRFTISRDNAKNSLYLQMNSLRAEDMAVYYCARGRYYFPWWGQGTL | |
| | VTVSS | |
| 64 | EVQLVESGGGLVQPGGSLRLSCAASGFTFSRNAMFWVRQAPGKGLEWVSGIGTGG | SCB-VH-91 |
| | ATSYADSVKGRFTISRDNAKNSLYLQMNSLRAEDTAVYYCARGRYYFDYWGQGTLV | |
| | TVSS | |
| 65 | EVQLVQSGGGLVHPGGSLRLSCAGSGFTFSRNAMFWVRQAPGKGLEWVSGIGTGG | SCB-VH-92 |
| | ATNYADSVKGRFTISRDNAKNSLYLQMNSLRAEDMAVYYCARGRYYFDYWGQGTL | |
| | VTVSS | |
| 66 | EVQLVESGGGLVQPGGSLRLSCAASGFTFSRNAMFWVRQAPGKGLEWVSGIGTGG | SCB-VH-93 |
| | ATSYADSVKGRFTISRDNAKNSLYLQMNSLRAEDTAVYYCARGRYYFPWWGQGTLV | |
| | TVSS | |
| 67 | QVQLVQSGAEVKKPGASVKVSCKASGYAFTSYYMHWARQAPGQGLEWMGIINPS | MEDI-1-VH |
| | GGSTSYAQKFQGRVTMTRDTSTSTVYMELSSLRSEDTAVYYCARGKWWLDYWGKG | |
| | TLVTVSS | |
| 68 | QSVLTQPPSVSAAPGQKVTISCSGGSSNIGNSYVSWYQQLPGTAPKLLIYDNNKRPS | MEDI-1-VL |
| | GIPDRFSGSKSGTSATLAITGLQTGDEADYYCGTWDTSLSANYVFGTGTKLTVL | |
| 69 | QVQLVQSGAEVKKPGASVKVSCKASGYAFTSYYMHWARQAPGQGLEWMGIINPS | MEDI-2-VH |
| | GGSTSYAQKFQGRVTMTRDTSTSTVYMELSSLRSEDTAVYYCARGKWWLYNWGKG | |
| | TLVTVSS | |
| 70 | QSVLTQPPSVSAAPGQKVTISCSGGSSNIGNSYVSWYQQLPGTAPKLLIYDNNKRPS | MEDI-2-VL |
| | GIPDRFSGSKSGTSATLAITGLQTGDEADYYCGTWDTSQPPNPLFGTGTKLTVL | |
| 71 | QVQLVQSGAEVKKPGASVKVSCKASGYAFTSYYMHWARQAPGQGLEWMGIINPS | MEDI-3-VH |
| | GGSTSYAQKFQGRVTMTRDTSTSTVYMELSSLRSEDTAVYYCARGKLLKNPWGKGT | |
| | LVTVSS | |
| 72 | QSVLTQPPSVSAAPGQKVTISCSGGSSNIGNSYVSWYQQLPGTAPKLLIYDNNKRPS | MEDI-3-VL |
| | GIPDRFSGSKSGTSATLAITGLQTGDEADYYCGTWFGTPASNYVFGTGTKLTVL | |
| 73 | QVQLVQSGAEVKKPGASVKVSCKASGYAFTSYYMHWARQAPGQGLEWMGIINPS | MEDI-4-VH |
| | GGSTSYAQKFQGRVTMTRDTSTSTVYMELSSLRSEDTAVYYCARGKWWLYNWGKG | |
| | TLVTVSS | |
| 74 | QSVLTQPPSVSAAPGQKVTISCSGGSSNIGNSYVSWYQQLPGTAPKLLIYDNNKRPS | MEDI-4-VL |
| | GIPDRFSGSKSGTSATLAITGLQTGDEADYYCGTWDTSSPPQPIFGTGTKLTVL | |
| 75 | QVQLVQSGAEVKKPGASVKVSCKASGYAFTSYYMHWARQAPGQGLEWMGIINPS | MEDI-5-VH |
| | GGSTSYAQKFQGRVTMTRDTSTSTVYMELSSLRSEDTAVYYCARGKWWLYDWGKG | |
| | TLVTVSS | |
| 76 | QSVLTQPPSVSAAPGQKVTISCSGGSSNIGNSYVSWYQQLPGTAPKLLIYDNNKRPS | MEDI-5-VL |
| | GIPDRFSGSKSGTSATLAITGLQTGDEADYYCGTWDTSSPPQPIFGTGTKLTVL | |
| 77 | QVQLVQSGAEVKKPGASVKVSCKASGYAFTSYYMHWARQAPGQGLEWMGIINPS | MEDI-6-VH |
| | GGSTSYAQKFQGRVTMTRDTSTSTVYMELSSLRSEDTAVYYCARGKYWMYDWGKG | |
| | TLVTVSS | |
| 78 | QSVLTQPPSVSAAPGQKVTISCSGGSSNIGNSYVSWYQQLPGTAPKLLIYDNNKRPS | MEDI-6-VL |
| | GIPDRFSGSKSGTSATLAITGLQTGDEADYYCGTWDTSTTYHPIFGTGTKLTVL | |
| 79 | QVQLVQSGAEVKKPGASVKVSCKASGYAFTSYYMHWARQAPGQGLEWMGIINPS | MEDI-7-VH |
| | GGSTSYAQKFQGRVTMTRDTSTSTVYMELSSLRSEDTAVYYCARGKWWWQYWGK | |
| | GTLVTVSS | |
| 80 | QSVLTQPPSVSAAPGQKVTISCSGGSSNIGNSYVSWYQQLPGTAPKLLIYDNNKRPS | MEDI-7-VL |
| | GIPDRFSGSKSGTSATLAITGLQTGDEADYYCGTWDTSSPPQPIFGTGTKLTVL | |
| 81 | QVQLVQSGAEVKKPGASVKVSCKASGYAFTSYYMHWARQAPGQGLEWMGIINPS | MEDI-8-VH |
| | GGSTSYAQKFQGRVTMTRDTSTSTVYMELSSLRSEDTAVYYCARGKWWWQYWGK | |
| | GTLVTVSS | |
| 82 | QSVLTQPPSVSAAPGQKVTISCSGGSSNIGNSYVSWYQQLPGTAPKLLIYDNNKRPS | MEDI-8-VL |
| | GIPDRFSGSKSGTSATLAITGLQTGDEADYYCGTWDTSTTYHPIFGTGTKLTVL | |
| 83 | QVQLVQSGAEVKKPGASVKVSCKASGYAFTSYYMHWARQAPGQGLEWMGIINPS | MEDI-9-VH |
| | GGSTSYAQKFQGRVTMTRDTSTSTVYMELSSLRSEDTAVYYCARGKWWLYNWGKG | |
| | TLVTVSS | |
| 84 | QSVLTQPPSVSAAPGQKVTISCSGGSSNIGNSYVSWYQQLPGTAPKLLIYDNNKRPS | MEDI-9-VL |
| | GIPDRFSGSKSGTSATLAITGLQTGDEADYYCGTWDTSTTMYPLFGTGTKLTVL | |
| 85 | QVQLVQSGAEVKKPGASVKVSCKASGYAFTSYYMHWARQAPGQGLEWMGIINPS | MEDI-10-VH |
| | GGSTSYAQKFQGRVTMTRDTSTSTVYMELSSLRSEDTAVYYCARGKWWLYDWGKG | |
| | TLVTVSS | |
| 86 | QSVLTQPPSVSAAPGQKVTISCSGGSSNIGNSYVSWYQQLPGTAPKLLIYDNNKRPS | MEDI-10-VL |
| | GIPDRFSGSKSGTSATLAITGLQTGDEADYYCGTWDTSTVLTPIFGTGTKLTVL | |
| 87 | QVQLVQSGAEVKKPGASVKVSCKASGYAFTSYYMHWARQAPGQGLEWMGIINPS | MEDI-11-VH |
| | GGSTSYAQKFQGRVTMTRDTSTSTVYMELSSLRSEDTAVYYCARGKWWFYDWGKG | |
| | TLVTVSS | |
| 88 | QSVLTQPPSVSAAPGQKVTISCSGGSSNIGNSYVSWYQQLPGTAPKLLIYDNNKRPS | MEDI-11-VL |
| | GIPDRFSGSKSGTSATLAITGLQTGDEADYYCGTWDTSPSMIPLFGTGTKLTVL | |
| 89 | QVQLVQSGAEVKKPGASVKVSCKASGYAFTSYYMHWARQAPGQGLEWMGIINPS | MEDI-12-VH |
| | GGSTSYAQKFQGRVTMTRDTSTSTVYMELSSLRSEDTAVYYCARGKWWFYDWGKG | |
| | TLVTVSS | |
| 90 | QSVLTQPPSVSAAPGQKVTISCSGGSSNIGNSYVSWYQQLPGTAPKLLIYDNNKRPS | MEDI-12-VL |
| | GIPDRFSGSKSGTSATLAITGLQTGDEADYYCGTWDTSTTMYPLFGTGTKLTVL | |
| 91 | QVQLVQSGAEVKKPGASVKVSCKASGYAFTSYYMHWARQAPGQGLEWMGIINPS | MEDI-13-VH |
| | GGSTSYAQKFQGRVTMTRDTSTSTVYMELSSLRSEDTAVYYCARGKWWLYDWGKG | |
| | TLVTVSS | |
| 92 | QSVLTQPPSVSAAPGQKVTISCSGGSSNIGNSYVSWYQQLPGTAPKLLIYDNNKRPS | MEDI-13-VL |
| | GIPDRFSGSKSGTSATLAITGLQTGDEADYYCGTWDTSTTLQPLFGTGTKLTVL | |
| 93 | QVQLVQSGAEVKKPGASVKVSCKASGYAFTSYYMHWARQAPGQGLEWMGIINPS | MEDI-14-VH |
| | GGSTSYAQKFQGRVTMTRDTSTSTVYMELSSLRSEDTAVYYCARGKWWLYNWGKG | |
| | TLVTVSS | |
| 94 | QSVLTQPPSVSAAPGQKVTISCSGGSSNIGNSYVSWYQQLPGTAPKLLIYDNNKRPS | MEDI-14-VL |
| | GIPDRFSGSKSGTSATLAITGLQTGDEADYYCGTWDTSPPTKPLFGTGTKLTVL | |
| 95 | QVQLVQSGAEVKKPGASVKVSCKASGYAFTSYYMHWARQAPGQGLEWMGIINPS | MEDI-15-VH |
| | GGSTSYAQKFQGRVTMTRDTSTSTVYMELSSLRSEDTAVYYCARGKWWLYNWGKG | |
| | TLVTVSS | |
| 96 | QSVLTQPPSVSAAPGQKVTISCSGGSSNIGNSYVSWYQQLPGTAPKLLIYDNNKRPS | MEDI-15-VL |
| | GIPDRFSGSKSGTSATLAITGLQTGDEADYYCGTWDTSTHRHPLFGTGTKLTVL | |
| 97 | QVQLVQSGAEVKKPGASVKVSCKASGYAFTSYYMHWARQAPGQGLEWMGIINPS | MEDI-16-VH |
| | GGSTSYAQKFQGRVTMTRDTSTSTVYMELSSLRSEDTAVYYCARGKWWLYNWGKG | |
| | TLVTVSS | |
| 98 | QSVLTQPPSVSAAPGQKVTISCSGGSSNIGNSYVSWYQQLPGTAPKLLIYDNNKRPS | MEDI-16-VL |
| | GIPDRFSGSKSGTSATLAITGLQTGDEADYYCGTWDTSTTYHPIFGTGTKLTVL | |
| 99 | QVQLVQSGAEVKKPGASVKVSCKASGYAFTSYYMHWARQAPGQGLEWMGIINPS | MEDI-17-VH |
| | GGSTSYAQKFQGRVTMTRDTSTSTVYMELSSLRSEDTAVYYCARGKWWWQHWGK | |
| | GTLVTVSS | |
| 100 | QSVLTQPPSVSAAPGQKVTISCSGGSSNIGNSYVSWYQQLPGTAPKLLIYDNNKRPS | MEDI-17-VL |
| | GIPDRFSGSKSGTSATLAITGLQTGDEADYYCGTWDTSPVDRPIFGTGTKLTVL | |
| 101 | QVQLVQSGAEVKKPGASVKVSCKASGYAFTSYYMHWARQAPGQGLEWMGIINPS | MEDI-18-VH |
| | GGSTSYAQKFQGRVTMTRDTSTSTVYMELSSLRSEDTAVYYCARGKWWWQHWGK | |
| | GTLVTVSS | |
| 102 | QSVLTQPPSVSAAPGQKVTISCSGGSSNIGNSYVSWYQQLPGTAPKLLIYDNNKRPS | MEDI-18-VL |
| | GIPDRFSGSKSGTSATLAITGLQTGDEADYYCGTWDTSTTPMPVFGTGTKLTVL | |
| 103 | QVQLVQSGAEVKKPGASVKVSCKASGYAFTSYYMHWARQAPGQGLEWMGIINPS | MEDI-19-VH |
| | GGSTSYAQKFQGRVTMTRDTSTSTVYMELSSLRSEDTAVYYCARGKWWWQHWGK | |
| | GTLVTVSS | |
| 104 | QSVLTQPPSVSAAPGQKVTISCSGGSSNIGNSYVSWYQQLPGTAPKLLIYDNNKRPS | MEDI-19-VL |
| | GIPDRFSGSKSGTSATLAITGLQTGDEADYYCGTWDTSTTYHPIFGTGTKLTVL | |
| 105 | QVQLVQSGAEVKKPGASVKVSCKASGYAFTSYYMHWARQAPGQGLEWMGIINPS | MEDI-20-VH |
| | GGSTSYAQKFQGRVTMTRDTSTSTVYMELSSLRSEDTAVYYCARGKYWMYDWGKG | |
| | TLVTVSS | |
| 106 | QSVLTQPPSVSAAPGQKVTISCSGGSSNIGNSYVSWYQQLPGTAPKLLIYDNNKRPS | MEDI-20-VL |
| | GIPDRFSGSKSGTSATLAITGLQTGDEADYYCGTWDTSTVWEWPFGTGTKLTVL | |
| 107 | QVQLVQSGAEVKKPGASVKVSCKASGYAFTSYYMHWARQAPGQGLEWMGIINPS | MEDI-21-VH |
| | GGSASYAQKFQGRVTMTRDTSTSTVYMELSSLRSEDTAVYYCARGKYWMYDWGK | |
| | GTLVTVSS | |
| 108 | QSVLTQPPSVSAAPGQKVTISCSGGSSNIGNSYVSWYQQLPGTAPKLLIYDNNKRPS | MEDI-21-VL |
| | GIPDRFSGSKSGTSATLAITGLQTGDEAVYFCGTWDTSTVWEWPFGTGTKLTVL | |
| 109 | QVQLVQSGAEVKKPGASVKVSCKASGYAFTSYYMHWARQAPGQGLEWMGIINPS | MEDI-22-VH |
| | GGSTSYAQKFQGRVTMTRDTSTSTVYMELSSLRSEDTAVYYCARGKYWMYDWGKG | |
| | TLVTVSS | |
| 110 | QPVLTQPPSVSAAPGQKVTISCSGGSSNIGNSYVSWYQQLPGTAPKLLIYDNNKRPS | MEDI-22-VL |
| | GIPDRFSGSKSGTSATLAITGLQTGDEADYFCGTWDTSTVWEWPFGTGTKLTVL | |
| 111 | QVQLVQSGAEVRKPGASVKVSCKASGYAFTSYYMHWARQAPGQGLEWMGIINPS | MEDI-23-VH |
| | GGSTSYAQKFQGRVTMTRDTSTSTVYMELSSLRSEDTAVYYCARGKYWMYDWGKG | |
| | TLVTVSS | |
| 112 | QSVLTQPPSVSAAPGQKVTISCSGGSSNIGNNYVSWYQQLPGTAPKLLIYDNNKRPP | MEDI-23-VL |
| | GIPDRFSGSKSGTSATLAITGLQTGDEADYYCGTWDTSTVWEWPFGTGTKLTVL | |
| 113 | QVQLVQSGAEVKKPGASVKVSCKASGYAFTSYYMHWARQAPGQGLEWMGIINPR | MEDI-24-VH |
| | GGSTSYAQKFQGRVTMTRDTSTSTVYMELSSLRSEDTAVYYCARGKYWMYDWGKG | |
| | TLVTVSS | |
| 114 | QSVLTQPPSVSAAPGQKVTISCSGGSSNIGNSYVSWYQQLPGTAPKLLIYDNNKRPS | MEDI-24-VL |
| | GIPDRFSGSKSGTSATLAITGLQTGDEADYFCGTWDTSTVWEWPFGTGTKLTVL | |
| 115 | QVQLVQSGAEVKKPGASVKVSCKASGYAFTSYYMHWARQAPGQGLEWMGIINPR | MEDI-25-VH |
| | GGSASYAQKFQGRVSMTRDTSTSTVYMELSSLRSEDTAVYYCARGKYWMYDWGK | |
| | GTLVTVSS | |
| 116 | QSVLTQPPSVSAAPGQKVTISCSGGSSNIGNSYVSWYQQLPGTAPKLLIYDNNKRPS | MEDI-25-VL |
| | GIPDRFSGSKSGTTATLAITGLQTGDEADYYCGTWVTSTVWEWPFGTGTKLTVL | |
| 117 | QVQLVQSGAEVKKPGASVKVSCKASGYAFTSYYMHWARQAPGQGLEWMGIINPS | MEDI-26-VH |
| | GGSTSYAQKFQGRVTMTRDTSTSTVYMELSSLRSEDTAVYYCARGKYWMYDWGKG | |
| | TLVTVSS | |
| 118 | QSVLTQPPSVSAAPGQKVTISCSGGSSNIGNSYVSWYQQLPGTAPKLLIYDNNKRPS | MEDI-26-VL |
| | GIPDRFSGSKSGTSATLAITGLQTGDEADYFCGTWDTSTVWEWPFGTGTKLTVL | |
| 119 | QVQLVQSGAEVRKPGASVKVSCKASGYAFTSYYMHWARQAPGQGLEWMGIINPS | MEDI-27-VH |
| | GGSTSYAQKFQGRVTMTRDTSTSTVYMELSSLRPEDTAVYYCARGKYWMYDWGK | |
| | GTQVTVSS | |
| 120 | QSVLTQPPLVSAAPGQKVTISCSGGSSNIGNSYVSWYQRLPGTAPKLLIYDNNKRPSG | MEDI-27-VL |
| | IPDRFSGSKSGTSATLAITGLQTGDEADYYCGTWDTSTVWEWPFGTGTKLTVL | |
| 121 | QVQLVQSGAEVKKPGASVKVSCKASGYAFTSYYMHWARQAPGQGLEWMGIINPS | MEDI-28-VH |
| | GGSTSYAQKFQGRVTMTRDTSTSTVYMELSSLRSEDTAVYYCARGKYWMYDWGN | |
| | GTLVTVSS | |
| 122 | LPVLTQPPSVSAAPGQKVTISCSGGSSSIGNSYVSWYQQLPGAAPKLLIYDNNKRPSG | MEDI-28-VL |
| | IPDRFSGFRSGTSATLAITGLQTGDEADYYCGTWDTSPVWEWPFGTGTKLTVL | |
| 123 | QVQLVQSGAEVKKPGASVKVSCKASGYAFTSYYMHWARQAPGQGLEWMGIINPS | MEDI-29-VH |
| | GGSTSYAQKFQGRVTMTRDTSTSTVYMELSSLRSEDTAVYYCARGKYWMYDWGKG | |
| | TRVTVSS | |
| 124 | QSVLTQPPSVSAAPGQKVTISCSGGSSNIGNSYVSWYQQLPGTAPKLLIYDNNKRPS | MEDI-29-VL |
| | GIPDRFSGSKSGTSATLAITGLQTGDEADYYCGTWDTSPVWEWPFGTGTKLTVL | |
| 125 | QVQLVQSGAEVKKPGASVKVSCKASGYAFTSYYMHWARQAPGQGLEWMGIINPS | MEDI-30-VH |
| | GGSTSYAQKFQGRVTMTRDTSTSTVYMELSSLRSEDTAVYYCARGKYWMYDWGKG | |
| | TLVTVSS | |
| 126 | QSVLTQPPSVSAAPGQKVTISCSGGSSNIGNSYVSWYQRLPGAAPKLLIYDNNKRPS | MEDI-30-VL |
| | GIPDRFSGSKSGTSATLAITGLQTGDEADYYCGTWDTSTVWEWPFGTGTKLTVL | |
| 127 | QVQLVQSGAEVKKPGASVKVSCKASGYAFTSYYMHWARQAPGQGLEWMGIINPS | MEDI-31-VH |
| | GGSTSYAQKFQGRVTMTRDTSTSTVYMELSSLRSEDTAVYYCARGKYWMYDWGKG | |
| | TLVTVSS | |
| 128 | QSVLTQPPSVSAAPGQKVTISCSGGSSSIGNSYVSWYQQLPGTAPKLLIYDNNKRPSG | MEDI-31-VL |
| | IPDRFSGSKSGTSATLAITGLQTGDEADYYCGTWATSPVWEWPFGTGTKLTVL | |
| 129 | QVQLVQSGAEVKKPGASVKVSCKASGYAFTSYYMHWARQAPGQGLEWMGIINPS | MEDI-32-VH |
| | GGSTSYAQKFQGRVTMTRDTSTSTVYMELSSLRSEDTAVYYCARGKYWMYDWGKG | |
| | TLVTVSS | |
| 130 | QSVLTQPPSVSAAPGQKVTISCSGGSSNIGNSYVSWYQQLPGTAPKLLIYDNNKRPS | MEDI-32-VL |
| | GIPDRFSGSKSGTSATLAITGLQTGDEADYFCGTWDTSTAWEWPFGTGTKLTVL | |
| 131 | QVQLVQSGAEEKKPGASVKVSCKASGYAFTSYYMHWARQAPGQGLEWMGIINPS | MEDI-33-VH |
| | GGSTSYAQKFQGRVTMTRDTSTSTVYMELSSLRSEDTAVYYCARGKYWMYDWGKG | |
| | TLVTVSS | |
| 132 | QSALTQPPSVSAAPGQKVTISCSGGSSNIGNSYVSWYQQLPGTAPKLLIYDNNKRPS | MEDI-33-VL |
| | GIPDRFSGSKSGTSATLAITGLQTGDEADYFCGTWDTSTVWEWPFGTGTKLTVL | |
| 133 | QVQLVQSGAEVKKPGASVKVSCKASGYAFTSYYMHWARQAPGQGLEWMGIINPS | MEDI-34-VH |
| | GGSTSYAQKFQGRVSMTRDTSTSTVYMELSSLRSEDTAVYYCARGKYWMYDWGKG | |
| | TLVTVSS | |
| 134 | QSVLTQPPSVSAAPGQKVTISCSGGSSNIGNSYVSWYQQLPGTAPKLLIYDNNKRPS | MEDI-34-VL |
| | GIPDRFSGSKSGTSATLAITGLQTGDEADYFCGTWDTSTVWEWPFGTGTKLTVL | |
| 135 | QVQLVQSGAEVKKPGASVKVSCKASGYAFTSYYMHWARQAPGQGLEWMGIINPS | MEDI-35-VH |
| | GGSTSYAQKFQGRVTMTRDTSTSTVYMELSSLRSEDTAVYYCARGKYWMYDWGKG | |
| | TLVTVSS | |
| 136 | QSVLTQPPSVSAAPGQKVTISCSGGSSNIGNSYVSWYQQLPGTAPKLLIYDNNKRPS | MEDI-35-VL |
| | GIPDRFSGSKSGTSATLAITGLQTGDEADYYCGTWDTSPVWEWPFGTGTKLTVL | |
| 137 | QVQLVQSGAEVKKPGASVKVSCKASGYAFTSYYMHWARQAPGQGLEWMGIINPS | MEDI-36-VH |
| | GGSASYAQKFQGRVTMTRDTSTSTVYMELSSLRSEDTAVYYCARGKYWMYDWGK | |
| | GTLVTVSS | |
| 138 | QSVLTQPPSVSAAPGQKVTISCSGGSSNIGNSYVSWYQQLPGTAPKLLIYDNNKRPS | MEDI-36-VL |
| | GIPDRFSGSKSGTSATLAITGLQTGDEADYYCGTWDSSTVWEWPFGTGTKLTVL | |
| 139 | QVQLVQSGAEVKKPGASVKVSCKASGYAFTSYYMHWARQAPGQGLEWMGIINPR | MEDI-37-VH |
| | GGSTSYAQKFQGRVAMTRDTSTSTVYMELSSLRPEDTAVYYCARGKYWMYDWGK | |
| | GTLVTVSS | |
| 140 | QSVLTQPPSVSAAPGQKVTISCSGGGSSIGNSYVSWYQQLPGTAPKLLIYDNNKRPS | MEDI-37-VL |
| | GVPDRFSGSKSGTSATLAITGLQTGDEADYYCGTWDTSPVWEWPFGTGTKLTVL | |
| 141 | QVQLVQSGAEVKKPGASVKVSCKASGYAFTSYYMHWARQAPGQGLEWMGIINPS | MEDI-38-VH |
| | GGSASYAQKFQGRVTMTRDTSTSTVYMELSSLRSEDTAVYYCARGKYWMYDWGK | |
| | GTLVTVSS | |
| 142 | QSVLTQPPSVSAAPGQKVTISCSGGSSNIGNSYVSWYQQLPGTAPKLLIYDNNKRPS | MEDI-38-VL |
| | GIPDRFSGSKSGTSATLAITGLQTGDEADYFCGTWDTSTVWEWPFGTGTKLTVL | |
| 143 | QVQLVQSGAEVKKPGASVKVSCKASGYAFTSYYMHWARQAPGQGLEWMGIINPR | MEDI-39-VH |
| | GGSTSYAQKFQGRVTMTRDTSTSTVYMELSSLRSEDTAVYYCARGKYWMYDWGKG | |
| | TLVTVSS | |
| 144 | QSVLTQPPSVSAAPGQKVTISCSGGSSNIGNSYVSWYQQLPGTAPKLLIYDNNKRPS | MEDI-39-VL |
| | GIPDRFSGSKSGTSATLAITGLQTGDEADYYCGTWDTSTAWEWPFGTGTKLTVL | |
| 145 | QVQLVQSGAEVKKPGASVKVSCKASGYAFTSYYMHWARQAPGQGLEWMGIINPS | MEDI-40-VH |
| | GGSTSYAQKFQGRVTMTRDTSTSTVYMELSSLRSEDTAVYYCARGKYWMYDWGKG | |
| | TLVTVSS | |
| 146 | QSVLTQPPSVSAAPGQKVTISCSGGSSNIGNSYVSWYQQLPGTAPKLLIYDNNKRPS | MEDI-40-VL |
| | GIPDRFSGSKSGTSATLAITGLQTGDEADYYCGTWDSSTVWEWPFGTGTKLTVL | |
| 147 | QVQLVQSGAEVRKPGASVKVSCKASGYAFTSYYMHWARQAPGQGLEWMGIINPS | MEDI-41-VH |
| | GGSTSYAQKFQGRVTMTRDTSTSTVYMELSSLRPEDTAVYYCARGKYWMYDWGK | |
| | GTLVTVSG | |
| 148 | QSVLTQPPSVSAAPGQKVTISCSGGSTNIGNSYVSWYQRLPGTAPKLLIYDNNKRPP | MEDI-41-VL |
| | GIPDRFSGSKSGTSATLAITGLQTGDEADYYCGTWDTSTVWEWPFGTGTKLTVL | |
| 149 | QVQLVQSGAEVKKPGASVKVSCKASGYAFTSYYMHWARQAPGQGLEWVGIINPSG | MEDI-42-VH |
| | GSTSYAQKFQGRVTMTRDTSTSTVYMELSSLRSGDTAVYYCARGKYWMYDWGKGT | |
| | LVTVSS | |
| 150 | QAVLTQPPSVSAAPGQKVTISCSGGSSNIGNSYVSWYQRLPGAAPKLLIYDNNKRPS | MEDI-42-VL |
| | GIPDRFSGSKSGTSATLAITGLQTGDEADYYCGTWDTSTGWEWPFGTGTKLTVL | |
| 151 | QVQLVQSGAEVKKPGASVKVSCKASGYAFTSYYMHWVRQAPGQGLEWMGIINPR | MEDI-37GL-VH |
| | GGSTSYAQKFQGRVTMTRDTSTSTVYMELSSLRSEDTAVYYCARGKYWMYDWGKG | |
| | TLVTVSS | |
| 152 | QSVLTQPPSVSAAPGQKVTISCSGGGSSIGNSYVSWYQQLPGTAPKLLIYDNNKRPS | MEDI-37GL-VL |
| | GIPDRFSGSKSGTSATLGITGLQTGDEADYYCGTWDTSPVWEWPFGTGTKLTVL | |
| 153 | EVQLLESGGGLVQPGGSLRLSCAVSGFTFSNYAMSWVRQAPGKGLEWVSAISSGGG | AJOU-1-VH |
| | NIYYADSVKGRFTISRDNSKNTLYLQMNSLRAEDTAVYYCAKLRRYFDYWGQGTLVT | |
| | VSS | |
| 154 | EVQLLESGGGLVQPGGSLRLSCAASGFTFSDYAMSWVRQAPGKGLEWVSAISSGGS | AJOU-2-VH |
| | SIYYADSVKGRFTISRDNSKNTLHLQMNSLRAEDTAVYYCARGPQRSATAVFDYWG | |
| | QGTLVTVSS | |
| 155 | EVQLLESGGGLVQPGGSLRLSCAASGFTFSNYAMSWVRQAPGKGLEWVSWISPNS | AJOU-3-VH |
| | GNIYYADSVKGRFTISRDNSKNTLYLQMNSLRAEDTAVYYCARRPLSAAWSHSSYYN | |
| | AMDVWGQGTLVTVSS | |
| 156 | EVQLLESGGGLVQPGGSLRLSCAASGFTFSGYAMSWVRQAPGKGLEWVSLISHSGS | AJOU-4-VH |
| | NTYYADSVKGRFTISRDNSKNTLYLQMNSLRAEDTAVYYCARPHRAFDYWGQGTLV | |
| | TVSS | |
| 157 | EVQLLESGGGLVQPGGSLRLSCAASGFTFSNYAMSWVRQAPGKGLEWVSGISHGS | AJOU-5-VH |
| | GSIYYADSVKGRFTISRDNSKNTLYLQMNSLRAEDTAVYYCARPHRAFDYWGQGTLV | |
| | TVSS | |
| 158 | EVQLLESGGGLVQPGGSLRLSCAASGFTFSNYAMSWVRQAPGKGLEWVSGISHGN | AJOU-6-VH |
| | GSIYYADSVKGRFTISRDNSKNTLYLQMNSLRAEDTAVYYCAKTGRHFDYWGQGTLV | |
| | TVSS | |
| 159 | EVQLLESGGGLVQPGGSLRLSCAASGFTFSNYAMSWVRQAPGKGLEWVSSISPSGS | AJOU-7-VH |
| | SIYYADSVKGRFTISRDNSKNTLYLQMNSLRAEDTAVYYCARSYRAFDYWGQGTLVT | |
| | VSS | |
| 160 | EVQLLESGGGLVQPGGSLRLSCAASGFTFSNYAMSWVRQAPGKGLEWVSAISPSGG | AJOU-8-VH |
| | SIYYADSVKGRFTISRDNSKNTLYLQMNSLRAEDTAVYYCARAKRAFDYWGQGTLVT | |
| | VSS | |
| 161 | EVQLLESGGGLVQPGGSLRLSCAASGFTFSNYAMSWVRQAPGKGLEWVSAISPGSG | AJOU-9-VH |
| | STYYADSVKGRFTISRDNSKNTLYLQMNSLRAEDTAVYYCAKFRRHFDYWGQGTLVT | |
| | VSS | |
| 162 | EVQLLESGGGLVQPGGSLRLSCAASGFTFSNYAMSWVRQAPGKGLEWVSAISSGGG | AJOU-10-VH |
| | NIYYADSVKGRFTISRDNSKNTLYLQMNSLRAEDTAVYYCARVHRAFDYWGQGTLV | |
| | TVSS | |
| 163 | EVQLLESGGGLVQPGGSLRLSCAASGFTFSNYAMSWVRQAPGKGLEWVSAITSSGR | AJOU-69-VH |
| | SIYYADSVKGRFTISRDNSKNTLYLQMNSLRAEDTAVYYCARVHRAFDYWGQGTLVT | |
| | VSS | |
| 164 | EVQLLESGGGLVQPGGSLRLSCAASGFTFSNYAMSWVRQAPGKGLEWVSAITSSGA | AJOU-70-VH |
| | NIYYADSVKGRFTISRDNSKNTLYLQMNSLRAEDTAVYYCARVHRAFDYWGQGTLV | |
| | TVSS | |
| 165 | EVQLLESGGGLVQPGGSLRLSCAASGFTFSNYAMSWVRQAPGKGLEWVSAITSSGG | AJOU-71-VH |
| | NIYYADSVKGRFTISRDNSKNTLYLQMNSLRAEDTAVYYCARVHRAFDYWGQGTLV | |
| | TVSS | |
| 166 | EVQLLESGGGLVQPGGSLRLSCAASGFTFSNYAMSWVRQAPGKGLEWVSAITAGG | AJOU-72-VH |
| | GSIYYADSVKGRFTISRDNSKNTLYLQMNSLRAEDTAVYYCARVHRAFDYWGQGTLV | |
| | TVSS | |
| 167 | EVQLLESGGGLVQPGGSLRLSCAASGFTFSRHAMAWVRQAPGKGLEWVSAITSSGR | AJOU-83-VH |
| | SIYYADSVKGRFTISRDNSKNTLYLQMNSLRAEDTAVYYCARVHRAFDYWGQGTLVT | |
| | VSS | |
| 168 | QSVLTQPPSASGTPGQRVTISCSGSSSNIGNNYVNWYQQLPGTAPKLLIYDNSHRPS | AJOU-33-VL |
| | GVPDRFSGSKSGTSASLAISGLRSEDEADYYCGTWDASLSAYVFGGGTKLTVL | |
| 169 | QSVLTQPPSASGTPGQRVTISCSGSSSNIGNNNVSWYQQLPGTAPKLLIYANSKRPS | AJOU-34-VL |
| | GVPDRFSGSKSGTSASLAISGLRSEDEADYYCGSWDDSLSAYVFGGGTKLTVL | |
| 170 | QSVLTQPPSAPGTPGQRVTISCTGSSSNIGSNSVNWYQQLPGTAPKLLIYDDSHRPS | AJOU-35-VL |
| | GVPDRFSGSKSGTSASLAISGLRSEDEADYYCDAWDSSLSAYVFGGGTKLTVL | |
| 171 | QSVLTQPPSASGTPGQRVTLSCTGSSSNIGSNYVSWYQQLPGTAPKLLIYADSQRPSG | AJOU-36-VL |
| | VPDRFSGSKSGTSASLAISGLRSEDEADYYCGTWDDSLSGYVFGGGTKLTVL | |
| 172 | QSVLTQPPSASGTPGQRVTISCSSSSSNIGSNYVSWYQQLPGTAPKLLIYSDSHRPSG | AJOU-37-VL |
| | VPDRFSGSKSGTSASLAISGLRSEDEADYYCGSWDYSLSAYVFGGGTKLTVL | |
| 173 | QSVLTQPPSASGTPGQRVTISCTGSSSNIGNNTVSWYQQLPGTAPKLLIYDNSHRPS | AJOU-38-VL |
| | GVPDRFSGSKSGTSASLAISGLQSEDEADYYCGSWDYSLSAYVFGGGTKLTVL | |
| 174 | QSVLTQPPSASGTPGQRVTISCTGSSSNIGNNDVNWYQQLPGTAPKLLIYYDSQRPS | AJOU-39-VL |
| | GVPDRFSGSKSGTSASLAISGLRSEDEADYYCATWDASLSAYVFGGGTKLTVL | |
| 175 | QSVLTQPPSASGTPGQRVTISCSGSSSNIGSNAVNWYQQLPGTAPKLLIYYDNQRPS | AJOU-40-VL |
| | GVPDRFSGSKSGTSASLAISGLRSEDEADYYCGTWDDSLNGYVFGGGTKLTVL | |
| 176 | QSVLTQPPSASGTPGQRVTISCSGSSSNIGNNAVTWYQQLPGTAPKLLIYDDSHRPS | AJOU-41-VL |
| | GVPDRFSGSKSGTSASLAISGLRSEDEADYYCGSWDYSLSAYVFGGGTKLTVL | |
| 177 | QSVLTQPPSASGTPGQRVTISCSGSSSNIGSNTFNWYQQLPGTAPKLLIYADSHRPSG | AJOU-42-VL |
| | VPDRFSGSKSGTSASLAISGLRSEDEADYYCGTWDYSLSGYVLGGGTKLTVL | |
| 178 | QSVLTQPPSASGTPGQRVTISCSGSSSNIGSNTFNWYQQLPGTAPKLLIYADSHRPSG | AJOU-77-VL |
| | VPDRFSGSKSGTSASLAISGLRSEDEADYYCGTWDYSLSGYVLGGGTKLTVL | |
| 179 | QSVLTQPPSASGTPGQRVTISCSGSSSNIGSNTFNWYQQLPGTAPKLLIYADSHRPSG | AJOU-78-VL |
| | VPDRFSGSKSGTSASLAISGLRSEDEADYYCGTWDYSLRGYVLGGGTKLTVL | |
| 180 | QSVLTQPPSASGTPGQRVTISCSGSSSNIGSNTFNWYQQLPGTAPKLLIYADSHRPSG | AJOU-79-VL |
| | VPDRFSGSKSGTSASLAISGLRSEDEADYYCGYWDYSLSGYVLGGGTKLTVL | |
| 181 | QSVLTQPPSASGTPGQRVTISCSGSSSNIGSNTFNWYQQLPGTAPKLLIYADSHRPSG | AJOU-80-VL |
| | VPDRFSGSKSGTSASLAISGLRSEDEADYYCGTWDYSLSGYVLGGGTKLTVL | |
| 182 | QSVLTQPPSASGTPGQRVTISCSGSSANSRTDGFNWYQQLPGTAPKLLIYADSHRPS | AJOU-86-VL |
| | GVPDRFSGSKSGTSASLAISGLRSEDEADYYCGTWDYSLSGYVLGGGTKLTVLG | |
| 183 | QSVLTQPPSASGTPGQRVTISCSGSAQFGSRDNFNWYQQLPGTAPKLLIYADSHRPS | AJOU-87-VL |
| | GVPDRFSGSKSGTSASLAISGLRSEDEADYYCGTWDYSLSGYVLGGGTKLTVLG | |
| 184 | QSVLTQPPSASGTPGQRVTISCSGSTKQMHNYQFNWYQQLPGTAPKLLIYADSHRP | AJOU-88-VL |
| | SGVPDRFSGSKSGTSASLAISGLRSEDEADYYCGTWDYSLSGYVLGGGTKLTVLG | |
| 185 | QSVLTQPPSASGTPGQRVTISCSGSLLRGENLQFNWYQQLPGTAPKLLIYADSHRPS | AJOU-89-VL |
| | GVPDRFSGSKSGTSASLAISGLRSEDEADYYCGTWDYSLSGYVLGGGTKLTVLG | |
| 186 | QSVLTQPPSASGTPGQRVTISCSGSPLFPDSGSFNWYQQLPGTAPKLLIYADSHRPSG | AJOU-90-VL |
| | VPDRFSGSKSGTSASLAISGLRSEDEADYYCGTWDYSLSGYVLGGGTKLTVLG | |
| 187 | QSVLTQPPSASGTPGQRVTISCSGSAALDLSPSFNWYQQLPGTAPKLLIYADSHRPSG | AJOU-91-VL |
| | VPDRFSGSKSGTSASLAISGLRSEDEADYYCGTWDYSLSGYVLGGGTKLTVLG | |
| 188 | QVQLVQSGAEVKKPGASVKVSCKASGYTFTNYGISWVRQAPGQGLEWMGWISVY | REGN-VH-3 |
| | NGKTNYAQKLQGRVTMTTDTSTTTAYMEMRSLRSDDTAVYYCARGSGYDLDYWG | |
| | QGTLVSVSS | |
| 189 | EVQLVESGGGLVQPGGSLRLSCAASGFTFSSFWMTWVRQAPGKGLEWVANIKQD | REGN-VH-19 |
| | GSEKYYVDSVKGRFTISRDNAKNSLYLQMNSLRAEDTAVYYCARDPGRTMVRGGIRY | |
| | YYGMDVWGQGTTVTVSS | |
| 190 | EVKLAESGGGLVQPGGSLRLSCAASGFTFSSHWMNWVRQAPGKGLEWVANIKQD | REGN-VH-35 |
| | GSDKYYVDSVKGRFTISRDNAKNSLYLQLNSLIAEDTAVYYCARDRGVRPPRGAFDIW | |
| | GQGTMVTVSS | |
| 191 | QVQLVQSGAEVKKPGASVKVSCKASGYTFNSYGISWVRQAPGQGLEWMGWIRTY | REGN-VH-51 |
| | NGNTNYAQKLQGRVTMTTDTSTSTAYMELRSLRSDDTAVYYCARDEARIVVAGTTP | |
| | YYYGMDVWGQGTTVTVSS | |
| 192 | QVQLVESGGGLVQPGGSLRLSCAVSGFTISDHYMSWIRQAPGKGLEWISYISSSGSKI | REGN-VH-67 |
| | YYADSVKGRFTISRDNAKNSLFLQMNSLRAEDTAVYYCARTRQLVGDYWGQGTLVT | |
| | VSS | |
| 193 | EVQLVESGGGLVQPGRSLRLSCAASGFTFDNYAMHWVRQAPGKGLEWVSGIRWN | REGN-VH-83 |
| | SGSIGYADSVKGRFTISRDNAKNSLYLQMNSLRAEDTALYYCAKEGGYSGYRPGPFFD | |
| | YWGQGTLVTVSS | |
| 194 | QVQLVQSGAEVKKPGASVKVSCKASGYTFTNYGISWVRQAPGQGLEWMGWISVY | REGN-VH-99 |
| | NGHTNYAQKLQGRVTMTTDTSTSTAYMELRSLRSDDTAVYYCARGSGYDFDSWGQ | |
| | GTLVTVSS | |
| 195 | QVQLVQSGAEVKKPGASVKVSCKASRYTFTSYDINWVRQATGQGLEWMGWMNP | REGN-VH-115 |
| | NSGNTGYAQKFQGRVTMTRNTSTSTAYMELSSLRSEDTAVYYCARVRRFFDYWGQ | |
| | GTLVTVSS | |
| 196 | QVQLVQSGPEVKKPGASVKVSCKASGYTFTNYGISWVRQAPGQGLEWMGWISVY | REGN-VH-147 |
| | NGNINYAQKLQGRVTMTTDTSTSTAYMDLRSLRSDDTAVYYCARGSGYDFDYWGQ | |
| | GTLVTVSS | |
| 197 | QVQLVQSGAEVKKPGASVKVSCKDSAYTFNRYGISWVRQAPGQGLEWMGWISAY | REGN-VH-163 |
| | TGNTVYAQKLQGRVTMTTDNSTSTAYMELRSLRSDDTAVYYCARDKSIFGVVRGFD | |
| | YWGQGTLVTVSS | |
| 198 | AIQMTQSPSSLSASVGDRVTITCRASQGIRNALGWYQQKPGKAPKLLIYAASSLQSG | REGN-VL-11 |
| | VPSRFSGSGSGTDFTLTFSSLQPEDFATYYCLQDFNYPYTFGQGTKLEIK | |
| 199 | DIQMTQSPSSVSASVGDRVTISCRASQGVSSWLAWYQQKPGNAPKLLISAASSIQSG | REGN-VL-27 |
| | VPSRFSGSGSGTDFTLTISSLOPEDFATYYCQQANSFPLTFGGGTKVEIK | |
| 200 | DIQMTQSPSSVSASVGDRVTITCRASQGISSWLAWYQQKPGKAPKLLIYAASSFQSG | REGN-VL-43 |
| | VPSRFSGSGSGTDFTLTISSLOPEDFATYFCQQANSFPLTFGGGTTVEIK | |
| 201 | DIQMTQSPSSVSASVGDRVTITCRASQDISIWLAWYQQSPGKAPKLLINVASRLQSG | REGN-VL-59 |
| | VPSRFSGSGSGTDFTLTINSLOPEDFVTYYCQQANSFPITFGQGTRLATK | |
| 202 | DIQLTQSPSFLSASVGDRVTITCWASQGISSYLAWYQQKPGKAPKLLIFAASTLQSGV | REGN-VL-75 |
| | PSRFSGSGSGTEFTLTISSLOPEDFATYYCQQLNSYPLTFGGGTKVEIR | |
| 203 | EIVMTQSPATLSVSPGERATLSCRASQSVNYNLAWYQHKPGQAPRLLIYGASTRATGI | REGN-VL-91 |
| | PARFSGSGSGTEFTLTISSLQSEDFAVYYCQQYNNWPLTFGGGTKVEIK | |
| 204 | AIQMTQSSSSLSASVGDRVTITCRASQAIRNALGWYQQKPGKAPKVLIYAASSLQSGI | REGN-VL-107 |
| | PSRFSGSGSGTDFTLTISSLOPEDFATYYCLQDYDYPYTFGQGTKLEIK | |
| 205 | DIQLTQSPSFLSASVGDRVTITCWASQGIISYLAWYQQKPGKAPKLLIYAASTLHSGVP | REGN-VL-123 |
| | SRFSGSGSGTEFTLTISSLOPEDFATYYCHQLKSYPITFGQGTRLEIK | |
| 206 | AIQMTQSPSSLSASVGDRVTITCRASQDIRNALGWYQQKPGKAPKLLIYAASSLQSG | REGN-VL-155 |
| | VPSRFSGSASGTDFTLTISSLQPEDFAAYYCLQDYNYPYTFGQGTKLEIK | |
| 207 | EIVMTQSPVTLSLSPGERATLPCRASQSVSSSLAWYQQKAGQSPRLLIYGASTRATGI | REGN-VL-171 |
| | PARFSGSGSGTEFTLTISNLQSEDFAVYYCQQYNNWPLTFGGGTKVEIK | |
| 208 | EVQLLESGGGLVQPGGSLRLSCAASGFTLSSYAMHWVRQAPGKGLEYVSGISSNGG | STSA-C27-VH |
| | STYYANSVKGRFTISRDNPKNTLFLQMSSLRAEDTAVYYCVRVKVGYRGGMDVWG | |
| | QGTTVTVSS | |
| 209 | EVQLLESGGGLVQPGGSLRLSCAASGFTLSSYAMHWVRQAPGKGLEYVSGISPSGSS | STSA-C27-6-33-VH |
| | TYYANSVKGRFTISRDNPKNTLFLQMSSLRAEDTAVYYCVRSKVRYRGGMDVWGQ | |
| | GTTVTVSS | |
| 210 | EVQLLESGGGLVQPGGSLRLSCAASGFTLSSYAMHWVRQAPGKGLEYVSGISPSGVS | STSA-C27-7-33-VH |
| | TYYANSVKGRFTISRDNPKNTLFLQMSSLRAEDTAVYYCVRVKVKYRGGMDVWGQ | |
| | GTTVTVSS | |
| 211 | EVQLLESGGGLVQPGGSLRLSCAASGFTLSSYAMHWVRQAPGKGLEYVSGISPTSGS | STSA-C27-24-56-VH |
| | TYYANSVKGRFTISRDNPKNTLFLQMSSLRAEDTAVYYCVRVKVRYRGGMDVWGQ | |
| | GTTVTVSS | |
| 212 | EVQLLESGGGLVQPGGSLRLSCAASGFTLSSYAMHWVRQAPGKGLEYVSGISPTGTS | STSA-C27-47-56-VH |
| | TYYANSVKGRFTISRDNPKNTLFLQMSSLRAEDTAVYYCVRVKGAYRGGMDVWGQ | |
| | GTTVTVSS | |
| 213 | EVQLLESGGGLVQPGGSLRLSCAASGFTLSSYAMHWVRQAPGKGLEYVSGISSSGSS | STSA-C27-33-33-VH |
| | TYYANSVKGRFTISRDNPKNTLFLQMSSLRAEDTAVYYCVRVKVAYRGGMDVWGQ | |
| | GTTVTVSS | |
| 214 | EVQLLESGGGLVQPGGSLRLSCAASGFTLSSYAMHWVRQAPGKGLEYVSGISPSSTS | STSA-C27-56-56-VH |
| | TYYANSVKGRFTISRDNPKNTLFLQMSSLRAEDTAVYYCVRVKVLYRGGMDVWGQ | |
| | GTTVTVSS | |
| 215 | EVQLLESGGGLVQPGGSLRLSCAASGFTLSSYAMHWVRQAPGKGLEYVSGISPSSAS | STSA-C27-78-78-VH |
| | TYYANSVKGRFTISRDNPKNTLFLQMSSLRAEDTAVYYCVRVKSKYRGGMDVWGQ | |
| | GTTVTVSS | |
| 216 | EVQLLESGGGLVQPGGSLRLSCAASGFTLSSYAMHWVRQAPGKGLEYVSGISGNSAS | STSA-C27-82-58-VH |
| | TYYANSVKGRFTISRDNPKNTLFLQMSSLRAEDTAVYYCVRVKLKYRGGMDVWGQG | |
| | TTVTVSS | |
| 217 | EVQLLESGGGLVQPGGSLRLSCAASGFTLSSYAMHWVRQAPGKGLEYVSGISHSGTS | STSA-C27-54-54-VH |
| | TYYANSVKGRFTISRDNPKNTLFLQMSSLRAEDTAVYYCVRVRVLYRGGMDVWGQ | |
| | GTTVTVSS | |
| 218 | EVQLLESGGGLVQPGGSLRLSCAASGFTLSSYAMHWVRQAPGKGLEYVSGISPSGVS | STSA-C27-36-36-VH |
| | TYYANSVKGRFTISRDNPKNTLFLQMSSLRAEDTAVYYCVRVKVKYRGGMDVWGQ | |
| | GTTVTVSS | |
| 219 | EVQLLESGGGLVQPGGSLRLSCAASGFTLSSYAMHWVRQAPGKGLEYVSGISSNGG | STSA-C27-53-53-VH |
| | STYYANSVKGRFTISRDNPKNTLFLQMSSLRAEDTAVYYCVRVFVRYRGGMDVWGQ | |
| | GTTVTVSS | |
| 220 | EVQLLESGGGLVQPGGSLRLSCAASGFTLSSYAMHWVRQAPGKGLEYVSGISPTSAS | STSA-C27-67-67-VH |
| | TYYANSVKGRFTISRDNPKNTLFLQMSSLRAEDTAVYYCVRVKGRYRGGMDVWGQ | |
| | GTTVTVSS | |
| 221 | EVQLLESGGGLVQPGGSLRLSCAASGFTLSSYAMHWVRQAPGKGLEYVSGISPTGGS | STSA-C27-55-55-VH |
| | TYYANSVKGRFTISRDNPKNTLFLQMSSLRAEDTAVYYCVRVKGRYRGGMDVWGQ | |
| | GTTVTVSS | |
| 222 | EVQLLESGGGLVQPGGSLRLSCAASGFTLSSYAMHWVRQAPGKGLEYVSGISHSGN | STSA-C27-59-59-VH |
| | STYYANSVKGRFTISRDNPKNTLFLQMSSLRAEDTAVYYCVRVKRRYRGGMDVWGQ | |
| | GTTVTVSS | |
| 223 | EVQLLESGGGLVQPGGSLRLSCAASGFTLSSYAMHWVRQAPGKGLEYVSGISPSSNS | STSA-C27-58-58-VH |
| | TYYANSVKGRFTISRDNPKNTLFLQMSSLRAEDTAVYYCVRVKVRYRGGMDVWGQ | |
| | GTTVTVSS | |
| 224 | EVQLLESGGGLVQPGGSLRLSCAASGFTLSSYAMHWVRQAPGKGLEYVSGISSSGSS | STSA-C27-52-52-VH |
| | TYYANSVKGRFTISRDNPKNTLFLQMSSLRAEDTAVYYCVRVKPAYRGGMDVWGQ | |
| | GTTVTVSS | |
| 225 | EVQLLESGGGLVQPGGSLRLSCAASGFTLSSYAMHWVRQAPGKGLEYVSGISYSSAS | STSA-C27-Y2-Y2-VH |
| | TYYANSVKGRFTISRDNPKNTLFLQMSSLRAEDTAVYYCVRVKVRYRGGMDVWGQ | |
| | GTTVTVSS | |
| 226 | ETTLTQSPDTLPLSPGDRASLSCRASQSVSSAYLAWYQQKPGQAPRLLIYGTSRRATG | STSA-C27-VL |
| | VPGRFSGSGSGTDFTLTISRLEPEDFAVYYCQLYGSSSVTFGQGTKLEIK | |
| 227 | EIVLTQSPGTLSLSPGERATLSCRASQGISSAYLAWYQQKPGQAPRLLIYGTSRRATGI | STSA-C27-6-33-VL |
| | PDRFSGSGSGTDFTLTISRLEPEDFAVYYCQLYGATSVTFGQGTKLEIK | |
| 228 | EIVLTQSPGTLSLSPGERATLSCRASQGISSAYLAWYQQKPGQAPRLLIYGTSRRATGI | STSA-C27-7-33-VL |
| | PDRFSGSGSGTDFTLTISRLEPEDFAVYYCQLYGATSVTFGQGTKLEIK | |
| 229 | EIVLTQSPGTLSLSPGERATLSCRASQSVSSAYLAWYQQKPGQAPRLLIYGTSRRATGI | STSA-C27-24-56-VL |
| | PDRFSGSGSGTDFTLTISRLEPEDFAVYYCQLYGASSVTFGQGTKLEIK | |
| 230 | EIVLTQSPGTLSLSPGERATLSCRASQSVSSAYLAWYQQKPGQAPRLLIYGTSRRATGI | STSA-C27-47-56-VL |
| | PDRFSGSGSGTDFTLTISRLEPEDFAVYYCQLYGASSVTFGQGTKLEIK | |
| 231 | EIVLTQSPGTLSLSPGERATLSCRASQGISSAYLAWYQQKPGQAPRLLIYGTSRRATGI | STSA-C27-33-33-VL |
| | PDRFSGSGSGTDFTLTISRLEPEDFAVYYCQLYGATSVTFGQGTKLEIK | |
| 232 | EIVLTQSPGTLSLSPGERATLSCRASQSVSSAYLAWYQQKPGQAPRLLIYGTSRRATGI | STSA-C27-56-56-VL |
| | PDRFSGSGSGTDFTLTISRLEPEDFAVYYCQLYGASSVTFGQGTKLEIK | |
| 233 | EIVLTQSPGTLSLSPGERATLSCRASQSISTAYLAWYQQKPGQAPRLLIYGTSRRATGIP | STSA-C27-78-78-VL |
| | DRFSGSGSGTDFTLTISRLEPEDFAVYYCQLYGASSVTFGQGTKLEIK | |
| 234 | EIVLTQSPGTLSLSPGERATLSCRASQDISSAYLAWYQQKPGQAPRLLIYGTSRRATGI | STSA-C27-82-58-VL |
| | PDRFSGSGSGTDFTLTISRLEPEDFAVYYCQLYGATSVTFGQGTKLEIK | |
| 235 | EIVLTQSPGTLSLSPGERATLSCRASQDVSSAYLAWYQQKPGQAPRLLIYGTSRRATGI | STSA-C27-54-54-VL |
| | PDRFSGSGSGTDFTLTISRLEPEDFAVYYCQLYGATSVTFGQGTKLEIK | |
| 236 | EIVLTQSPGTLSLSPGERATLSCRASQNISTAYLAWYQQKPGQAPRLLIYGTSRRATGI | STSA-C27-36-36-VL |
| | PDRFSGSGSGTDFTLTISRLEPEDFAVYYCQLYGATSVTFGQGTKLEIK | |
| 237 | EIVLTQSPGTLSLSPGERATLSCRASQDASNAYLAWYQQKPGQAPRLLIYGTSRRATG | STSA-C27-53-53-VL |
| | IPDRFSGSGSGTDFTLTISRLEPEDFAVYYCQLYGSSSVTFGQGTKLEIK | |
| 238 | EIVLTQSPGTLSLSPGERATLSCRASQGVSSAYLAWYQQKPGQAPRLLIYGTSRRATGI | STSA-C27-67-67-VL |
| | PDRFSGSGSGTDFTLTISRLEPEDFAVYYCQLYGRSSVTFGQGTKLEIK | |
| 239 | EIVLTQSPGTLSLSPGERATLSCRASQNISTAYLAWYQQKPGQAPRLLIYGTSRRATGI | STSA-C27-55-55-VL |
| | PDRFSGSGSGTDFTLTISRLEPEDFAVYYCQLYGTSSVTFGQGTKLEIK | |
| 240 | EIVLTQSPGTLSLSPGERATLSCRASQSVSTAYLAWYQQKPGQAPRLLIYGTSRRATGI | STSA-C27-59-59-VL |
| | PDRFSGSGSGTDFTLTISRLEPEDFAVYYCQLYGATSVTFGQGTKLEIK | |
| 241 | EIVLTQSPGTLSLSPGERATLSCRASQDISSAYLAWYQQKPGQAPRLLIYGTSRRATGI | STSA-C27-58-58-VL |
| | PDRFSGSGSGTDFTLTISRLEPEDFAVYYCQLYGATSVTFGQGTKLEIK | |
| 242 | EIVLTQSPGTLSLSPGERATLSCRASQGVSTAYLAWYQQKPGQAPRLLIYGTSRRATGI | STSA-C27-52-52-VL |
| | PDRFSGSGSGTDFTLTISRLEPEDFAVYYCQLYGATSVTFGQGTKLEIK | |
| 243 | EIVLPQSPGTLSLSPGERATLSCRASQGVSSAYLAWYQQKPGQAPRLLIYGTSRRATGI | STSA-C27-Y2-Y2-VL |
| | PDRFSGSGSGTDFTLTISRLEPEDFAVYYCQLYGSTSVTFGQGTKLEIK | |
| 244 | EVQLVESGGGLVQPKGSLKLSCAASGFTFNTYGMHWVRQAPGKGLEWVAHIRSKS | Y0188-1 VH |
| | SNYATYYADSVKDRFTISRDDSQSMLYLQMNNLKTEDTAMYYCVRWFRAMDYWG | |
| | QGTSVTVSS | |
| 245 | EVQLIESGGGLVQPKGSLKLSCAASGFTFNMYAMDWVRQAPGKGLEWVARIRSKG | Y0188-2 VH |
| | SNFETNYADSVKDRFTISRDDSQSMVYLQMINLKTEDTAMYYCVRHRGGAWFAYW | |
| | GQGTLVSVSA | |
| 246 | QVQLVETGGGLVRPGNSLKLSCVTSGFTFSNYRMHWLRQPPGKRLEWIAVITVKSN | Y0188-3 VH |
| | NYGANYAESVKGRFAISRDDSKSSVYLEMNRLREEDTATYFCSRERAYGNPFDYWG | |
| | QGTTLTVSS | |
| 247 | EVQLVESGGGLVQPKGSLKLSCAASGFTFNMYAMNWVRQAPGQGLEWVARIRSKS | Y0188-4 VH |
| | NNYATYYADSVKDRFIISRDDSESMVYLQMSNLRAADTAMYYCVRHLRAMDYWG | |
| | QGTSVTVSS | |
| 248 | EVQLVESGGGLVQPKGSLKLSCAASGFSFNMYAMNWVRQAPGKGLEWVARIRTKS | Y0188-6 VH |
| | NHYSTYYADSVKDRFTISRDDSASMFYLQMNNLKTEDTAMYFCVRHLRAMDYWG | |
| | QGTSVTVSS | |
| 249 | EVQLIESGGGLVQPKGSLKLSCAASGFTFNMYAMDWVRQAPGKGLEWVARIRSKG | Y0188-8 VH |
| | SNFETNYADSVKDRFTISRDDSQSMVYLQMNNLKTEDTAMYYCVRHRGGAWFAY | |
| | WGQGTLVTVSA | |
| 250 | EVQLVESGGGLVRPKGSLKLSCAASGFSFNTYAMNWVRQAPGKGLEWIVWIRSKSH | Y0188-9 VH |
| | NYATYYADSVKDRFTISRDDSESMLYLQMNNLKTEDTAMYYCVRHLRAMDYWGQ | |
| | GTSVTVSS | |
| 251 | EVRLVESGGGLVQPKGSLKLSCEASGFSFNMYAMNWVRQAPGKGLEWITHIRSKSN | Y0188-10 VH |
| | NYATYYADSVKDRFIISRDDSESMVYLQMNNLKTEDTAMYYCVRLLRALDYWGQGT | |
| | SVTVSS | |
| 252 | EVQLVESGGGLVQPKGSLKLSCAASGFTFNMYGMHWVRQAPGKGLEWVAHIRSKS | Y0188-14 VH |
| | SNYATYYADSVKDRLTISRDDSQSMLYLQMNNLKTEDTAMYYCVRWFRAMDYWG | |
| | QGTSVTVSS | |
| 253 | EVQLVESGGGLVKPGGSLRLSCAASGFTFSMYGMHWVRQAPGKGLEWVGHIRSKS | HV3-15-14 VH |
| | SNYATYYADSVKDRFTISRDDSKNTLYLQMNSLKTEDTAVYYCTTWFRAMDYWGQ | |
| | GTLVTVSS | |
| 254 | EVQLVESGGGLVQPGGSLRLSCAASGFTFSMYGMHWVRQAPGKGLEWVSHIRSKS | HV3-48-14 VH |
| | SNYATYYADSVKDRFTISRDNAKNSLYLQMNSLRAEDTAVYYCARWFRAMDYWGQ | |
| | GTLVTVSS | |
| 255 | EVQLVESGGGLVQPGGSLKLSCAASGFTFSMYGMHWVRQASGKGLEWVGHIRSKS | HV3-73*2-14 VH |
| | SNYATYYADSVKDRFTISRDDSKNTAYLQMNSLKTEDTAVYYCTRWFRAMDYWGQ | |
| | GTLVTVSS | |
| 256 | EVQLVESGGGLVQPGGSLRLSCAASGFTFSMYGMHWVRQAPGKGLEWVGHIRSKS | HV3-72-14 VH |
| | SNYATYYADSVKDRFTISRDDSKNSLYLQMNSLKTEDTAVYYCARWFRAMDYWGQ | |
| | GTLVTVSS | |
| 257 | EVQLVESGGGLVQPGGSLRLSCAASGFTFSMYGMHWVRQAPGKGLEWVSHIRSKS | Y01-14 VH |
| | SNYATYYADSVKDRFTISRDNAKNSLYLQMNSLRAEDTAVYYCARWFRAMDYWGQ | |
| | GTLVTVSS | |
| 258 | EVQLVESGGGLEQPGGSLRLSCAGSGFTFRMYGMHWVRQAPGKGLEWVSHIRSKS | 162-14 VH |
| | SNYATYYADSVKDRFTISRDNSKNTLYLQMNSLRAEDTAVYYCAKWFRAMDYWGQ | |
| | GTTVTVSS | |
| 259 | EVQLVESGGGLVQPGGSLKLSCAASGFTFSMYGMHWVRQASGKGLEWVGHIRSKS | VH73-14 VH |
| | SNYATYYADSVKDRFTISRDDSKNTAYLQMNSLKTEDTAVYYCTRWFRAMDYWGQ | |
| | GTTVTVSS | |
| 260 | DIVMTQSHKFMSTSVGDRVSITCKASQDVSTAVAWYQEKPGQSPKLLIYWASTRHT | Y0188-1 VL |
| | GVPDRFTGSGSGTDYTLTISSVQAEDLALYYCQQHYSTPLTFGAGTKLELK | |
| 261 | DIVVTQSPASLAVSLGQRATISCRASKSVSTSGYSYMHWYQQKPGQPPKLLIYLASNL | Y0188-2 VL |
| | ESGVPARFSGSGSGTDFTLNIHPVEEEDVAIYYCQHSRELPLTFGAGTKLELK | |
| 262 | DIQMTQSPSSLSASLGERVSLTCRASQEISGYLSWLQQKPDGTIKRLIYAASTLDSGVP | Y0188-3 VL |
| | KRFSGSRSGSDYSLTISSLESEDFADYYCLQYGSYPYTFGGGTKLEIK | |
| 263 | DIVLTQSPASLTVSLGQRATISCRASKSVSTSGYSYMHWYQQKPGQPPKLLIYLASNLE | Y0188-4 VL |
| | SGVPARFSGSGSGTDFTLNIHPVEEEDAATYYCQHSRELPITFGSGTKLEIK | |
| 264 | DIVLTQSPASLVVSLGQRATISCRASQSVSTSGYSYMHWYQQKPGQPPKLLIYLASNV | Y0188-6 VL |
| | QSGVPARFSGSGSGTDFTLNIHPVEEEDVATYYCHHNRDLPFTFGSGTKLEIK | |
| 265 | DIVVTQSPASLAVSLGQRATISCRASKSVSTSGYSYMHWYQQKPGQPPKLLIYLASNL | Y0188-8 VL |
| | ESGVPARFSGSGSGTDFTLNIHPVEEEDVAIYYCQHSRELPLTFGAGTKLELK | |
| 266 | DIVLTQSPASLAVSLGQRATISCRASKSVSASGYSYMHWYQQKPGQPPKLLIYLASNL | Y0188-9 VL |
| | QSGVPARFSGSGSGTDFTLNIHPVEEEDAATYYCQHSRELPPTFGGGTKLEIK | |
| 267 | DIVLTQSPASLAVFLGQRATISCRASKSVSTSGYSYMHWYQQKAGQPPKLLIYLASNL | Y0188-10 VL |
| | ESGVPARFSGSGSGTDFTLNIHPVEEEDAATYYCHHSRELPITFGSGTKLEMK | |
| 268 | DIVMTQSHKFMSTSVGDRVSITCKASQDVSTAVAWYQEKPGQSPKLLIYWASTRHT | Y0188-14 VL |
| | GVPDRFTGSGSGTDYTLTISSVQAEDLALYYCQQHYSTPLTFGAGTKLELK | |
| 269 | EIVLTQSPGTLSLSPGERATLSCKASQDVSTAVAWYQQKPGQAPRLLIYWASTRHTGI | Y01-14 VL |
| | PDRFSGSGSGTDFTLTISRLEPEDFAVYYCQQHYSTPLTFGQGTKVEIK | |
| 270 | DIVMTQSPLSLPVTPGEPASISCKASQDVSTAVAWYLQKSGQSPQLLIYWASTRHTG | 164-14 VL |
| | VPDRFSGSGSGTDFTLKISRVEAEDVGFYYCQQHYSTPLTFGQGTKLEIK | |
| 271 | DIVMTQSPDSLAVSLGERATINCKASQDVSTAVAWYQQKPGQPPKLLIYWASTRHT | KV4-14 VL |
| | GVPDRFSGSGSGTDFTLTISSLQAEDVAVYYCQQHYSTPLTFGGGTKVEIK | |
| 272 | DIQMTQSPSSLSASVGDRVTITCKASQDVSTAVAWYQQKPGKVPKLLIYWASTRHT | KV1-27-14 VL |
| | GVPSRFSGSGSGTDFTLTISSLOPEDVATYYCQQHYSTPLTFGGGTKVEIK | |
| 273 | DIQLTQSPSFLSASVGDRVTITCKASQDVSTAVAWYQQKPGKAPKLLIYWASTRHTG | KV1-9-14 VL |
| | VPSRFSGSGSGTEFTLTISSLOPEDFATYYCQQHYSTPLTFGGGTKVEIK | |
| 274 | DIQMTQSPSSLSASVGDRVTITCKASQDVSTAVAWYQQKPGKAPKLLLYWASTRHT | KV1-NL1-14 VL |
| | GVPSRFSGSGSGTDYTLTISSLQPEDFATYYCQQHYSTPLTFGGGTKVEIK | |
| 275 | AIRMTQSPFSLSASVGDRVTITCKASQDVSTAVAWYQQKPAKAPKLFIYWASTRHTG | KV1D-43-14 VL |
| | VPSRFSGSGSGTDYTLTISSLOPEDFATYYCQQHYSTPLTFGGGTKVEIK | |

What is claimed is:

1. A method of treating, preventing, or ameliorating at least one symptom of eosinophilic esophagitis (EoE) in a subject<12 years of age, the method comprising administering to the subject one or more doses of an interleukin-4 receptor (IL-4R) antagonist, wherein the IL-4R antagonist is an anti-IL-4R antibody, or an antigen-binding fragment thereof, that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence of LGS, and the LCDR3 comprises the amino acid sequence of SEQ ID NO:8.

2. The method of claim 1, wherein the subject is 1 year old and <12 years old.

3. The method of claim 1 or 2, wherein prior to the onset of treatment with the IL-4R antagonist the subject has an intraepithelial eosinophilic infiltration peak cell count≥15 eos/hpf as measured by endoscopic biopsy in at least two of the proximal esophageal region, mid esophageal region, and distal esophageal region.

4. The method of any one of claims 1 to 3, wherein the subject has been previously treated with a swallowed topical corticosteroid and/or a proton pump inhibitor (PPI).

5. The method of any one of claims 1 to 4, wherein the subject is unresponsive, inadequately responsive, or intolerant to treatment with a swallowed topical corticosteroid and/or a PPI, or wherein standard of care treatment is contraindicated.

6. The method of any one of claims 1 to 5, wherein the subject has a concomitant atopic disease.

7. The method of claim 6, wherein the concomitant atopic disease is a food allergy, atopic dermatitis, asthma, chronic rhinosinusitis, allergic rhinitis, or allergic conjunctivitis.

8. The method of any one of claims 1 to 7, wherein the subject has a body weight≥5 kg.

9. The method of any one of claims 1 to 8, wherein the subject has a body weight<60 kg.

10. The method of any one of claims 1 to 9, wherein prior to the onset of treatment the subject:
has a baseline peak intraepithelial eosinophilic cell count≥70 eos/hpf;
has a baseline mean intraepithelial eosinophilic cell count≥50 eos/hpf;
has a baseline EoE Endoscopic Reference Score (EoE-EREFS) score of at least 6; and/or
has a baseline serum total IgE level of at least 400 IU/L.

11. The method of any one of claims 1 to 10, wherein the subject is selected on the basis of not exhibiting one or more exclusion criteria selected from the group consisting of:
(a) having a body weight<5 kg;
(b) having a body weight≥60 kg;
(c) having eosinophilic gastroenteritis, hypereosinophilic syndrome, or eosinophilic granulomatosis with polyangiitis (Churg-Strauss syndrome);
(d) having a history of Crohn's disease, ulcerative colitis, celiac disease, or prior esophageal surgery;
(e) having an esophageal stricture unable to be passed with a standard, diagnostic, upper endoscope or having an esophageal stricture that requires dilation;
(f) treatment with a swallowed topical corticosteroid within the previous 8 weeks;
(g) prior treatment with subcutaneous immunotherapy (SCIT), unless on a stable SCIT maintenance dose for at least 1 year;
(h) prior treatment with sublingual immunotherapy (SLIT), epicutaneous immunotherapy (EPIT), or oral immunotherapy (OIT);
(i) treatment with a systemic immunosuppressant or immunomodulating drug within the previous 3 months;
(j) initiation or change of a food-elimination diet regimen within the previous 6 weeks;
(k) initiation, discontinuation, or change in the dosage regimen of a PPI, leukotriene inhibitor, nasal corticosteroid, or inhaled corticosteroid within the previous 8 weeks;
(l) treatment with a live (attenuated) vaccine within the previous 4 weeks;
(m) having active H. pylori, a helminthic infection, an active parasitic infection, or a chronic or acute infection requiring treatment with systemic antibiotics, antivirals, or antifungals;
(n) having a known or suspected immunodeficiency disorder;
(o) having a hepatic disease; and
(p) having a platelet level<100×10³/μL, a neutrophil level≤1.5×10³/μL, or an estimated glomerular filtration rate (eGFR)<30 mL/min/1.73 m².

12. The method of any one of claims 1 to 11, wherein the anti-IL-4R antibody or antigen-binding fragment thereof comprises a heavy chain variable region (HCVR) comprising the amino acid sequence of SEQ ID NO:1 and comprises a light chain variable region (LCVR) comprising the amino acid sequence of SEQ ID NO:2.

13. The method of any one of claims 1 to 12, wherein the anti-IL-4R antibody comprises a heavy chain comprising the amino acid sequence of SEQ ID NO:9 and a light chain comprising the amino acid sequence of SEQ ID NO:10.

14. The method of any one of claims 1 to 13, wherein the IL-4R antagonist is dupilumab.

15. The method of any one of claims 1 to 14, wherein the IL-4R antagonist is administered at a dose of about 50 mg to about 600 mg.

16. The method of any one of claims 1 to 15, wherein the subject has a body weight kg, wherein:
for a subject weighing≥kg to <15 kg, the IL-4R antagonist is administered at a dose of about 100 mg Q2W, about 200 mg Q3W, or about 200 mg Q4W;
fora subject weighing≥15 kg to <30 kg, the IL-4R antagonist is administered at a dose of about 200 mg Q2W or about 300 mg Q4W;
for a subject weighing≥30 kg to <60 kg, the IL-4R antagonist is administered at a dose of about 300 mg Q2W or about 200 mg Q2W; and/or
for a subject weighing≥60 kg, the IL-4R antagonist is administered at a dose of about 300 mg QW or about 300 mg Q2W.

17. The method of any one of claims 1 to 15, wherein the subject has a body weight kg, wherein:
for a subject weighing≥5 kg to <15 kg, the IL-4R antagonist is administered at a dose of about 100 mg Q2W or about 200 mg Q3W;
fora subject weighing≥15 kg to <30 kg, the IL-4R antagonist is administered at a dose of about 200 mg Q2W;
for a subject weighing≥30 kg to <60 kg, the IL-4R antagonist is administered at a dose of about 300 mg Q2W; and/or
for a subject weighing≥60 kg, the IL-4R antagonist is administered at a dose of about 300 mg QW.

18. The method of any one of claims 1 to 15, wherein the subject has a body weight kg, wherein:
for a subject weighing≥5 kg to <15 kg, the IL-4R antagonist is administered at a dose of about 200 mg Q3W;
fora subject weighing≥15 kg to <30 kg, the IL-4R antagonist is administered at a dose of about 200 mg Q2W;
for a subject weighing≥30 kg to <40 kg, the IL-4R antagonist is administered at a dose of about 300 mg Q2W; and/or
for a subject weighing≥40 kg, the IL-4R antagonist is administered at a dose of about 300 mg QW.

19. The method of any one of claims 1 to 16, wherein the subject weighs≥5 kg to <15 kg.

20. The method of claim 19, wherein the IL-4R antagonist is administered at a dose of about 100 mg Q2W.

21. The method of claim 19, wherein the IL-4R antagonist is administered at a dose of about 200 mg Q3W.

22. The method of claim 19, wherein the IL-4R antagonist is administered at a dose of about 200 mg Q4W.

23. The method of any one of claims 1 to 16, wherein the subject weighs≥15 kg to <30 kg.

24. The method of claim 23, wherein the IL-4R antagonist is administered at a dose of about 200 mg Q2W.

25. The method of claim 23, wherein the IL-4R antagonist is administered at a dose of about 300 mg Q4W.

26. The method of any one of claims 1 to 16, wherein the subject weighs≥30 kg to <60 kg.

27. The method of claim 26, wherein the IL-4R antagonist is administered at a dose of about 300 mg Q2W.

28. The method of claim 26, wherein the IL-4R antagonist is administered at a dose of about 200 mg Q2W.

29. The method of any one of claims 1 to 16, wherein the subject weighs≥60 kg.

30. The method of claim 29, wherein the IL-4R antagonist is administered at a dose of about 300 mg QW.

31. The method of claim 29, wherein the IL-4R antagonist is administered at a dose of about 300 mg Q2W.

32. The method of any one of claim 1 to 15 or 18, wherein the subject weighs≥30 kg to <40 kg and the IL-4R antagonist is administered at a dose of about 300 mg Q2W.

33. The method of any one of claim 1 to 15 or 18, wherein the subject weighs≥40 kg and the IL-4R antagonist is administered at a dose of about 300 mg QW.

34. The method of any one of claims 1 to 15, wherein the IL-4R antagonist is administered at a dose of about 100 mg to about 300 mg every week or every two weeks.

35. The method of any one of claims 1 to 15, wherein the IL-4R antagonist is administered at a dose of about 100 mg to about 300 mg every three weeks.

36. The method of any one of claims 1 to 35, wherein the IL-4R antagonist is administered subcutaneously.

37. The method of any one of claims 1 to 36, wherein the IL-4R antagonist is administered in combination with a second therapeutic agent or therapy.

38. The method of claim 37, wherein the second therapeutic agent or therapy is diet management.

39. The method of claim 37, wherein the second therapeutic agent or therapy is a proton pump inhibitor (PPI).

40. The method of any one of claims 1 to 39, wherein treatment with the IL-4R antagonist for at least 16 weeks results in:
a decrease in peak esophageal intraepithelial eosinophil count;
a reduction in the severity and/or extent of disease as measured by Eosinophilic Esophagitis-Histology Scoring System (EoE-HSS);
an improvement in esophageal anatomical characteristics as measured by Eosinophilic Esophagitis-Endoscopic Reference Score (EoE-EREFS);
an increase in body weight for age percentile; and/or
a decrease in a normalized enrichment score (NES) for a type 2 inflammation panel and/or an EoE diagnostic panel of genes.

41. The method of claim 40, wherein treatment with the IL-4R antagonist for 16 weeks results in a peak esophageal intraepithelial eosinophil count of ≤6 eos/hpf, and/or a decrease in peak esophageal intraepithelial eosinophil count of at least 75%.

42. The method of any one of claims 1 to 41, wherein the IL-4R antagonist is contained in a container selected from the group consisting of a glass vial, a syringe, a pre-filled syringe, a pen delivery device, and an autoinjector.

43. The method of claim 42, wherein the IL-4R antagonist is contained in a pre-filled syringe.

44. The method of claim 43, wherein the pre-filled syringe is a single-dose pre-filled syringe.

45. The method of claim 42, wherein the IL-4R antagonist is contained in an autoinjector.

46. The method of claim 42, wherein the IL-4R antagonist is contained in a pen delivery device.
